(19) United States
(12) Patent Application Publication
(10) Pub. No.: US 2021/0164993 A1
(43) Pub. Date: Jun. 3, 2021

(54) Methods and Systems for Improving Skin Condition

(71) Applicant: Pathway Skin, Inc., San Diego, CA (US)

(72) Inventors: Michael Nova, San Diego, CA (US); Manuel Villa, San Diego, CA (US); Cindy Wang, San Diego, CA (US); Sean Li, San Diego, CA (US); Anja Kammesheidt, San Diego, CA (US)

(21) Appl. No.: 17/167,197

(22) Filed: Feb. 4, 2021

Related U.S. Application Data
(63) Continuation of application No. 15/774,941, filed on May 9, 2018, now abandoned.
(60) Provisional application No. 62/253,548, filed on Nov. 10, 2015.

(51) Int. Cl. G01N 33/68; C12Q 1/6883

(52) U.S. Cl. CPC G01N 33/6881; C12Q 1/6883; G01N 2800/24; A61B 5/441; C12Q 2600/106; C12Q 2600/156; G01N 2800/52

(57) ABSTRACT

This present application generally relates to methods and systems that allow for the establishment of personalized skin care regimen for an individual based upon the individual's genetic profile comprising biomarkers genetically associated with skin phenotypic attributes and/or skin nutritional conditions. In particular, kits and methods are disclosed for determining an individual's genetic profile, which may be used to select an appropriate therapeutic/dietary regimen or lifestyle recommendation based at least in part on the biomarkers used, weights applied thereto, and the resulting likelihood of the individual to exhibit a plurality of skin phenotypic attributes. Such a personalized skin care regimen is advantageous as compared to traditional skin care programs.

### CROSS-REFERENCE TO RELATED APPLICATIONS

This application is a continuation of U.S. application Ser. No. 15/774,941, filed May 9, 2018, which is a nationalization of PCT Application No. PCT/US2016/061418, filed Nov. 10, 2016, which claims the benefit of and priority to U.S. provisional patent application Ser. No. 62/253,548, filed Nov. 10, 2015, the entirety of each of which is incorporated herein by reference.

### BACKGROUND OF THE INVENTION

### Technical Field

The present application generally relates to systems and methods for assessing skin condition, including systems and methods for determining the likelihood of an individual to exhibit one or more skin phenotypic attributes, and for selecting a skin care regimen appropriate for the individual based at least in part on the individual's genetic profile.

### Background and Relevant Art

Covering the entire human body, skin is considered the largest and most visible organ of the human body, and functions as a shield from various types of external stimuli, damage, as well as from dehydration. For example, in addition to being a protective barrier to external insults (e.g., heat, chemicals, radiation, and microorganisms including bacteria, viruses, and microfungi), skin is involved in thermoregulation, inhibits dehydration, and performs sensory functions. Skin is also a bioreactor that produces various hormones and lipids that enter the body's circulation. A variety of immune cells function in skin as a first line of defense against bacterial or viral invasion and to maintain immune surveillance in skin and nearby body tissues. As such, skin is also among the tissues most exposed to environmental stresses, hazards, and pathogens. For these reasons, establishment and maintenance of good skin health is important to overall human health.

It has been widely reported that skin functioning and skin attractiveness are dependent on nutrition. This is particularly evidenced by the development of skin lesions in response to nutritional deficiencies. In these situations, dietary supplementation with the deficient vitamins, minerals, or essential fatty acids improves skin conditions. In the past decades, as modern nutritional science has continued to develop new insights into the relation between food intake and skin health, there has been growing interest in the role of diet, specific food ingredients, and supplements in reducing the risk of skin health issues. In many instances, specific positive effects of food ingredients on skin conditions have proved to be biologically relevant and consequently allow for claims on products containing these functional ingredients, resulting in the development of new functional foods for optimal skin condition.

Skin health is also important for aesthetic reasons, as many people are increasingly especially concerned about the appearance of their skin. It is widely believed that a healthy skin appearance can be maintained by a combination of cleaning, nutrition, and application of therapeutic and cosmetic products. Often, individuals employ trial-and-error techniques to identify skin care products (and doses thereof) that produce a desirable skin appearance. However, overuse of skin care products can degrade skin health and appearance. Accordingly, there is a growing need for more precise methods for identifying dietary, therapeutic or cosmetic compositions (and suitable amounts of such compositions) that will enhance the health and appearance of an individual's skin. These methods would preferably be tailored to identify useful compositions and dosages for individuals.

In addition, skin care products and regimens currently used to improve skin quality and health are typically administered based upon the physically displayed symptoms of the individual and are not focused on determining the underlying causes of such conditions or causes of the change in appearance. Without considering underlying or causative factors involved in skin quality, health and appearance, skin care treatment is often not effective, especially for preventing skin health related conditions. In particular, when a skin disorder, disease or change in appearance has already developed when treatment is implemented, such treatment is typically not commenced until physical symptoms are already displayed. Therefore, a need remains for methods to determine the health of an individual's skin and propensity toward deleterious skin changes and degeneration by identifying factors, including genetic factors, which contribute to skin health so that steps can be taken to prevent, relieve, or treat skin disorders, diseases, and other types of skin health related conditions.

Further, there is a need for novel methods and systems for establishing and computing a personalized skin care that take into account an individual's genetic profile and his/her likely susceptibility to various skin health related conditions. There is also a need for methods and systems for assessing individual's skin health or predisposition to develop skin health related issues. Such assessment could be used to identify types and amounts of therapeutic and/or preventive regimens that can be used to alleviate, inhibit, or prevent skin health conditions.

Accordingly, there are a number of disadvantages in the state of the art, for skin health management.

### SUMMARY OF INVENTION

In one aspect, disclosed herein are methods for determining a likelihood of an individual to exhibit one or more skin phenotypic attributes. In one implementation, the method comprises providing a biological sample. The biological sample can have a genotype. For instance, the genotype of the sample can be a genotype of an individual or organism from which the biological sample was acquired or provided. In one or more embodiments, the biological sample can comprise or contain nucleic acid and/or protein. The biological sample can comprise a fluid sample, which may be, for example, saliva, blood, semen, urine, or other bodily fluid. In at least one embodiment, the biological sample can comprise saliva or epithelial cells obtained by buccal swab. The method can further include determining at least a portion of the genotype by identifying genetic variations associated with the skin phenotypic attributes. The skin phenotypic attributes can comprise one or more skin nutritional conditions and/or one or more skin health characteristics. The genetic variations can comprise a first set of preselected genetic variations and, optionally, a second set of preselected genetic variations. Each member of the first set of preselected genetic variations can be genetically associated with the one or more skin nutritional conditions and each member of the optional second set of preselected genetic variations can be genetically associated with the one or more skin health characteristics. In some embodiments, identifying genetic variations can comprise identifying nucleic acid (e.g., DNA and/or RNA) variations or protein variations, preferably as compared to a control. The control can comprise a whole or partial (consensus) genome and/or proteome for a species or other classification of an individual or organism.

The method can further include generating a personalized biomarker profile for the individual based on the identified genetic variations. The personalized biomarker profile can reflect or be based on the identified genetic variations. The method can further include determining the likelihood of the individual or organism to exhibit the skin phenotypic attributes based at least in part upon the personalized biomarker profile. For instance, the likelihood can be determined or calculated based on one or more of the number and type of the identified genetic variations, a weight or weighing factor given or applied to the identified genetic variations, the strength of the association between the identified genetic variations and the skin phenotypic attributes, personal and/or family history of the skin phenotypic attributes, environmental contributions to the skin phenotypic attributes, and so forth.

In one or more implementations of methods described herein at least one of the one or more skin nutritional conditions is selected from the group consisting of: folate level, folic acid level, Vitamin A level, Vitamin B2 level, Vitamin B6 level, Vitamin B12 level, Vitamin B3 level, Vitamin C level, Vitamin D level, Vitamin E level, omega-3 fatty acid level, omega-6 fatty acid level, and combinations thereof. In the same foregoing method or yet another method, the first set of preselected genetic variations comprises biomarkers mapped within one or more genes selected from the group consisting of: SLC23A1, MTHFR, NBPF3, FUT2, BCMO1 (BCO1), FADS1, GC genes, and the intergenic region near APOA5, which may, additionally, or alternatively, include the biomarkers of the first set of preselected genetic variations being selected from the group consisting of: rs2282679, rs33972313, rs1801133, rs1801131, rs4654748, rs602662, rs7501331, rs12934922, rs174547, rs12272004, and combinations thereof.

In one implementation of the disclosed methods, at least one of the one or more skin health characteristics is selected from the group consisting of: skin aging, skin tone, skin photoaging (optionally including skin aging and skin tone), skin texture and elasticity, skin moisture factor, skin inflammation and allergy risk, skin oxidation protection, skin glycation, and combinations thereof. In the same foregoing method or yet another method, the second set of preselected genetic variations comprises biomarkers mapped within one or more genes selected from the group consisting of: MCIR, TYR, SLC45A2 (MATP), SLC24A5, Intergenic ASIP Region, HERC2, IRF4, EXOC2, STXBP5L, 6p25.3 Region, AIP, NCOA6, ACE, HIF1A, ELN, SRPX, HMCN1, TMEM18, MTHFR, AQP3, FLG, HLA-C, IL12B, IL23R, TNIP1, IL13, the intergenic region between HLA-DRA and BTNL2, the intergenic region between PRELID2 and KCTD16, TNFAIP3, SOD2, GPX1, CA, NQO1, GLO1, and AGER, which may, additionally, or alternatively, include the biomarkers of the second set of preselected genetic variations being selected from the group consisting of: rs1805005, rs2228479, rs885479, rs1805007, rs1805008, rs1805009, rs11547464, rs1110400, rs1805006, rs1393350, rs1126809, rs1042602, rs16891982, rs26722, rs1426654, rs2555364, rs1015362, rs4911414, rs12913832, rs12203592, rs12210050, rs322458, rs1540771, rs1799750, rs4911442, rs1799752, rs4646994, rs11549465, rs7787362, rs35318931, rs10798036, rs7594220, rs1801133, rs1801131, rs558269137, rs17553719, rs61816761, rs150597413, rs397507563, rs12191877, rs2082412, rs2201841, rs17728338, rs20541, rs763035, rs111314066, rs610604, rs138726443, 1249insG (HGMD CI083373), rs374588791 (7264G>T), rs200519781, rs121909626, rs540453626 (8666C>G), rs578153418 (8667C>A), rs761212672 (9887C>A), S2889X (HGMD CX082304), rs4880, rs1050450, rs1001179, rs1800566, rs2917666, rs1130534, rs1049346, rs1800624, rs1800625, rs2070600, and combinations thereof.

The methods of the present application may individually or in some combination further include the biomarkers of the second set of preselected genetic variations being genetically associated with skin photoaging (including skin aging and skin tone) and being mapped within one or more genes selected from the group consisting of: MCIR, TYR, SLC45A2 (MATP), SLC24A5, ASIP Region, HERC2, IRF4, EXOC2, STXBP5L, 6p25.3 Region, AIP1, and NCOA6; the biomarkers of the second set of preselected genetic variations being genetically associated with skin texture and elasticity and being mapped within one or more genes selected from the group consisting of: ACE, HIF1A, ELN, SRPX HMCN1, TMEM8, and MTHFR; the biomarkers of the second set of preselected genetic variations being genetically associated with skin moisture factor and being mapped within one or more genes selected from the group consisting of: AQP3 and FLG; the biomarkers of the second set of preselected genetic variations being genetically associated with skin inflammation and allergy and being mapped within one or more genes selected from the group consisting of: FLG, HLA-C, IL12B, IL23R, TNIP1, IL13, MTHFR, the intergenic region between HLA-DRA and BTNL2, the intergenic region between PRELID2 and KCTD16, and TNFAIP3; and/or the biomarkers of the second set of preselected genetic variations being genetically associated with skin oxidation protection or skin glycation risk and being mapped within one or more genes selected from the group consisting of: SOD2, GPXJ, CAT, NQO1, GLO1, and AGER.

In one or more implementations, the first set of preselected genetic variations comprises biomarkers mapped within one or more genes selected from the group consisting of: SLC23A, MTHFR, NBPF3, FUT2, BCMO1, FADS1, GC genes, and the intergenic region near APOA5, and wherein the second set of preselected genetic variations comprises: a first subset of preselected genetic variations comprising biomarkers that are genetically associated with skin photoaging (including skin aging and skin tone) and are mapped within one or more genes selected from the group consisting of: MCIR, TYR, SLC45A2 (MATP), SLC24A5, ASIP Region, HERC2, IRF4, EXOC2, STXBP5L, 6p25.3 Region, MMP1, and NCOA6; a second subset of preselected genetic variations comprising biomarkers that are genetically associated with skin texture and elasticity and are mapped within one or more genes selected from the group consisting of: ACE, HIF1A, ELN, SRPX, HMCN1, TMEM18, and MTHFR; a third subset of preselected genetic variations comprising biomarkers that are genetically associated with skin moisture factor and are mapped within one or more genes selected from the group consisting of: AQP3 and FLG; a fourth subset of preselected genetic variations comprising biomarkers that are genetically associated with skin inflammation and allergy and are mapped within one or more genes selected from the group consisting of: FLG, HLA-C, IL12B, IL23R, TNIP1, IL13, MTHFR, the intergenic region between HLA-DRA and BTNL2, the intergenic region between PRELID2 and KCTD16, and TNFAIP3; and a fifth subset of preselected genetic variations comprising biomarkers that are genetically associated with skin oxidation protection or skin glycation and are mapped within one or more genes selected from the group consisting of: SOD2, GPX1, CAT, NQO1, GLO1, and AGER.

Methods of the present disclosure may further include the act of determining the likelihood of the individual to exhibit the one or more skin phenotypic attributes is further based on one or more criteria selected from the group consisting of: family history, general medical physiological measures, cholesterol levels, blood pressure, heart rate, growth hormone levels, insulin sensitivity, obesity, body weight, triglyceride levels, red blood cells, bone density, CD scan results, mRNA expression profiles, methylation profiles, protein expression profiles, and enzyme activity.

In some implementations of the methods disclosed herein, said act of identifying genetic variations comprises identifying a plurality of genetic variations associated with the one or more skin phenotypic attributes; assigning a weight to each genetic variation of the plurality of genetic variations, the weight comprising an aggregate value of one or more criteria, the one or more criteria selected form the group consisting of: nucleotide sequence homology, expression level, enzyme activity, relative synteny among the preselected biomarkers, family history, ontological relevance, quality of supporting research, and degree of phenotypic significance; and selecting at least a first and a second genetic variation from the plurality of genetic variations based on the results of weighting each genetic variation, wherein the first genetic variation comprises a member of the first set of preselected genetic variations and the second genetic variation comprises a member of the second set of preselected genetic variations.

In one or more implementations, the method may further comprise generating a personalized genetic profile report that contains genotypic information relevant to the individual's likelihood of exhibiting the one or more skin phenotypic attributes and providing a personalized skin care regimen and a personalized nutritional regimen based on the determined likelihood of the individual to exhibit the one or more skin phenotypic attributes. In some embodiments, the personalized skin care regimen and the personalized nutritional regimen comprise one or more selections of adaptive intervention selected from a type and duration of physical exercise, a type and duration of lifestyle counseling, a type and dosing of skin protective products, a type and dosing of skin health medications, a type and dosing of food, and a type and dosing of nutritional supplements.

In one or more embodiments, at least one of the genetic variations is a nucleic acid based genetic variation selected from the group consisting of: a genetic mutation, a gene amplification, a gene rearrangement, a deletion, an insertion, an InDel mutation, a single nucleotide polymorphism (SNP), an epigenetic alteration, a splicing variant, an RNA/protein overexpression, and an aberrant RNA/protein expression, and combinations thereof. In some embodiments, the at least a portion of the genotype from the biological sample is determined by performing an analytical assay comprising one or more of nucleic acid sequencing, polypeptide sequencing, restriction digestion, capillary electrophoresis, nucleic acid amplification-based assays, nucleic acid hybridization assay, comparative genomic hybridization, real-time PCR, quantitative reverse transcription PCR (qRT-PCR), PCR-RFLP assay, HPLC, mass-spectrometric genotyping, fluorescent in-situ hybridization (FISH), next generation sequencing (NGS), or a combination thereof. In some embodiments the analytical assay is an allele-specific polymerase chain reaction or NGS. In some embodiments, the at least a portion of the genotype from the biological sample is determined by performing an antibody-based assay comprising one or more of ELISA, immunohistochemistry, western blotting, mass spectrometry, flow cytometry, protein-microarray, immunofluorescence, multiplex detection assay, or combinations thereof.

Methods of the present application include methods for selecting a personalized skin care regimen for an individual, comprising: receiving a biological sample from the individual; determining at least a portion of a genotype from the biological sample by identifying genetic variations associated with skin phenotypic attributes, the skin phenotypic attributes comprising one or more skin nutritional conditions and one or more skin health characteristics, and the genetic variations comprising a first set of preselected genetic variations and a second set of preselected genetic variations, each member of the first set of genetic variations being genetically associated with one or more skin nutritional conditions and each member of the second set of genetic variations being genetically associated with one or more skin health characteristics; generating a personalized biomarker profile for the individual based on the identified genetic variations; assigning a plurality of weights to the identified genetic variations, the plurality of weights being based on one or more criteria selected from the group consisting of: nucleotide sequence homology, expression level, enzyme activity, relative synteny among the preselected biomarkers, family history, ontological relevance, quality of supporting research, and degree of phenotypic significance; determining a likelihood of the individual to exhibit the skin phenotypic attributes based at least in part on the personalized biomarker profile and the plurality of weights; and selecting a personalized skin care regimen appropriate for the individual based at least in part on the determined likelihood of the individual to exhibit the skin phenotypic attributes.

The foregoing method may, in some implementations further comprise reporting a relative level of risk of exhibiting each of the one or more skin phenotypic attributes, wherein the relative level of risk comprises one of a high risk, an increased risk, a reduced risk, or a normal risk. Additionally, or alternatively, the method may further comprise administering to the individual the selected personalized skin care regimen.

Kits are disclosed herein. For example, a kit of the present disclosure comprises genotyping reagents, the genotyping reagents comprising a first set of molecular probes specific to a first set of preselected genetic variations, each member of the first set of preselected genetic variations being genetically associated with one or more skin nutritional conditions; and a second set of molecular probes specific to a second set of preselected genetic variations, each member of the second set of preselected genetic variations being genetically associated with one or more skin health characteristics. In some embodiments, the first set and the second set of molecular probes are individually selected from the group consisting of: primers, fluorescent oligonucleotide probes, and antibodies.

In one or more embodiments, the kit may include the first set of preselected genetic variations comprising biomarkers mapped within one or more genes selected from the group consisting of: SLC23A, MTHFR, NBPF3, FUT2, BCMO1, FADS1, GC genes, and the intergenic region near APOA5, and the second set of preselected genetic variations comprising biomarkers that genetically associate with skin photoaging (including skin aging and skin tone) and are mapped within one or more genes selected from the group consisting of: MCIR, TYR, SLC45A2 (MATP), SLC24A5, ASIP Region, HERC2, IRF4, EXOC2, STXBP5L, 6p25.3 Region, MMP1, and NCOA6.

In one or more embodiments of the present disclosure, the kits include the first set of preselected genetic variations comprising biomarkers mapped within one or more genes selected from the group consisting of: SLC23A1, MTHFR, NBPF3, FUT2, BCMO1, FADS1, GC genes, and the intergenic region near APOA5, and the second set of preselected genetic variations comprising a first subset of preselected genetic variations comprising biomarkers that are genetically associated with skin photoaging (including skin aging and skin tone) and are mapped within one or more genes selected from the group consisting of: MCIR, TYR, SLC45A2 (MATP), SLC24A5, ASIP Region, HERC2, IRF4, EXOC2, STXBP5L, 6p25.3 Region, MMP1, and NCOA6; a second subset of preselected genetic variations comprising biomarkers that are genetically associated with skin texture and elasticity and are mapped within one or more genes selected from the group consisting of: ACE, HIF1A, ELN, SRPX HMCN1, TMEM8, and MTHFR; a third subset of preselected genetic variations comprising biomarkers that are genetically associated with skin moisture factor and are mapped within one or more genes selected from the group consisting of: AQP3 and FLG; a fourth subset of preselected genetic variations comprising biomarkers that are genetically associated with skin inflammation and allergy and are mapped within one or more genes selected from the group consisting of: FLG, HLA-C, IL12B, IL23R, TNIP1, IL13, MTHFR, the intergenic region between HLA-DRA and BTNL2, the intergenic region between PRELID2 and KCTD16, and TNFAIP3; and a fifth subset of preselected genetic variations comprising biomarkers that are genetically associated with skin oxidation protection or skin glycation and are mapped within one or more genes selected from the group consisting of: SOD2, GPXJ, CAT, NQO1, GLO1, and AGER.

Various systems are provided in the present disclosure. For example, a computer system for generating and displaying a personalized genetics profile, comprises one or more processors, and one or more computer-readable storage media having stored thereon computer-executable instructions that are executable by the one or more processors to cause the computer system to determine the likelihood of an individual to exhibit one or more skin phenotypic attributes, the computer-executable instructions including instructions that are executable to cause the computer system to perform at least the following: receive sequence data of a user sample, the sequence data comprising at least a portion of a user genotype; identify a plurality of loci in the sequence data corresponding to a first set of preselected genetic variations and a second set of preselected genetic variations, each member of the first set of preselected genetic variations being genetically associated with one or more skin nutritional conditions and each member of the second set of preselected genetic variations being genetically associated with one or more skin health characteristics; determine a genotype for each locus of the plurality of loci; based on one or more criteria associated with the genotype for each locus or for the locus itself, apply a weight to each of the one or more genetic variations corresponding to the genotyped plurality of loci; calculate a score for at least one of the one or more skin phenotypic attributes based on an aggregated weighted value of genotyped loci corresponding to the at least one of the one or more phenotypic attributes, the score corresponding to the individual's likelihood of exhibiting the at least one of the one or more skin phenotypic attributes; and generate and display a personalized genetics profile report comprising the one or more genetic variations corresponding to the genotyped plurality of loci and the score for the at least one of the one or more phenotypic attributes.

Additional embodiments and implementations of the present disclosure include methods for determining the likelihood of an individual to exhibit one or more skin phenotypic attributes, including (a) acquiring knowledge of the occurrence of one or more genetic variations associated with each member of a first set and a second set of preselected biomarkers in the individual, wherein each member of the first set of biomarkers is genetically associated with one or more skin nutritional conditions and each member of the second set of biomarkers is genetically associated with one or more skin phenotypic attributes; (b) generating a personalized biomarker profile for the individual from the acquired knowledge; and (c) determining the status of the individual's nutritional skin health and the likelihood of the individual to exhibit the one or more skin phenotypic attributes based at least in part upon the personalized biomarker profile.

In one aspect, some embodiments disclosed herein relate to methods for identifying a skin care regimen for an individual. The methods include (a) selecting an individual in need of a skin care regimen; (b) acquiring knowledge of the occurrence of one or more genetic variations associated with each member of a first set and a second set of preselected biomarkers in the individual, wherein each member of the first set of biomarkers is genetically associated with one or more skin nutritional conditions and each member of the second set of biomarkers is genetically associated with one or more skin phenotypic attributes; (c) generating a personalized biomarker profile for the individual from the acquired knowledge; (d) determining the status of skin nutritional health of the individual and the likelihood of the individual to exhibit the one or more skin phenotypic attributes based at least in part on the acquired knowledge; and (e) identifying a skin care regimen appropriate for the individual based at least in part upon the determined status of skin nutritional health and the determined likelihood of the individual to exhibit the one or more skin phenotypic attributes.

In another aspect, some embodiments disclosed herein relate to methods for administering a personalized skin care regimen to an individual. The methods include (a) identifying an individual in need of a skin care regimen; (b) acquiring knowledge of the occurrence of one or more genetic variations associated with each member of a first set and a second set of preselected biomarkers in the individual, wherein each member of the first set of biomarkers is genetically associated with one or more skin nutritional conditions and each member of the second set of biomarkers is genetically associated with one or more skin phenotypic attributes; (c) generating a personalized biomarker profile for the individual from the acquired knowledge; (d) customizing a skin care regimen appropriate for the individual based at least in part on the personalized biomarker profile; and (e) administering the customized skin care regimen to the individual.

In another aspect, some embodiments disclosed herein relate to kits for assessing skin health of an individual to exhibit one or more skin phenotypic attributes. The kits include reagents for assessing the occurrence of one or more genetic variations associated with each member of a first set and a second set of preselected biomarkers in the individual, wherein each member of the first set of biomarkers is genetically associated with one or more skin nutritional conditions and each member of the second set of biomarkers is genetically associated with one or more skin phenotypic attributes.

In yet another aspect, some embodiments disclosed herein relate to methods for recommending a personalized skin care regimen for an individual. The methods include (a) identifying an individual in need of a skin care regimen; (b) acquiring knowledge of the occurrence of one or more genetic variations associated with each member of a first set and a second set of preselected biomarkers in the individual, wherein each member of the first set of biomarkers is genetically associated with one or more skin nutritional conditions and each member of the second set of biomarkers is genetically associated with one or more skin phenotypic attributes, whereby generating a personalized biomarker profile for the individual from the acquired knowledge; (c) assigning, based at least in part on the personalized biomarker profile, a relative marker score to each of the one or more skin phenotypic attributes indicating whether the individual has an enhanced, diminished, or average likelihood of exhibiting the skin phenotypic attribute; (d) generating a personalized genetic profile report that comprises genetic information relevant to the individual's likelihood of exhibiting the one or more skin phenotypic attributes and consistent with the assigned marker scores; and (e) recommending a personalized skin care regimen to the individual based on the personalized profile report.

In a further aspect, some embodiments disclosed herein relate to a genetics-based system for skin care management. The system according to this aspect includes (a) a logic processor; (b) a genetic scanner communicatively coupled to the logic processor; (c) a stored program code that is executable by the logic processor; and (d) a report engine communicatively coupled to the logic processor. In such system, the reports produced by the report engine depend upon results from execution of the program code, wherein the program code configures the logic processor to receive from the genetic scanner information input pertaining to an individual's personalized genetic profile comprising a preselected set of biomarkers in order to assign a relative risk score to the individual based at least in part on the personalized biomarker profile, whereby determining the likelihood of the individual to exhibit one or more skin phenotypic attributes as indicated by the assigned relative risk score.

In yet a further aspect, some embodiments disclosed herein relate to a non-transitory computer readable medium. The computer readable medium according to this aspect contains executable instructions that when executed cause a processor to perform operations including: (a) receiving an individual's personalized genetic profile of a first set and a second set of biomarkers in the individual, wherein each member of the first set of biomarkers is genetically associated with one or more skin nutritional conditions and each member of the second set of biomarkers is genetically associated with one or more skin phenotypic attributes; (b) assigning, based at least in part on the personalized biomarker profile, a relative biomarker score to each of the one or more skin nutritional conditions and the one or more skin phenotypic attributes, each biomarker score indicating whether the individual has an enhanced, diminished, or average risk of the likelihood of exhibiting the skin phenotypic attributes or the one or more skin nutritional conditions; and (c) outputting a personalized skin care regimen for the individual based upon the assigned risk scores. In some embodiments, the assigning of the relative biomarker score is further based on one or more criteria selected from the group consisting of family history, general medical physiological measures, cholesterol levels, blood pressure, heart rate, growth hormone levels, insulin sensitivity, obesity, body weight, triglyceride levels, red blood cells, bone density, CD scan results, mRNA expression profiles, methylation profiles, protein expression profiles, and enzyme activity, or a combination thereof.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

Additional features and advantages of the disclosure will be set forth in the description which follows, and in part will be obvious from the description, or may be learned by the practice of the disclosure. The features and advantages of the disclosure may be realized and obtained by means of the instruments and combinations particularly pointed out in the appended claims. These and other features of the present disclosure will become more fully apparent from the following description and appended claims, or may be learned by the practice of the disclosure as set forth hereinafter.

### BRIEF DESCRIPTION OF DRAWINGS

In order to describe the manner in which the above recited and other advantages and features of the disclosure can be obtained, a more particular description of the disclosure briefly described above will be rendered by reference to specific embodiments thereof, which are illustrated in the appended drawings. It is appreciated that these drawings depict only typical embodiments of the disclosure and are not therefore to be considered to be limiting of its scope. The disclosure will be described and explained with additional specificity and detail through the use of the accompanying drawings in which:

FIG. 1 is a flow diagram illustrating a non-limiting example of the interaction of an individual and a healthcare provider in a system according to some embodiments disclosed herein.

FIG. 2 is a flow diagram illustrating of a non-limiting example of the method for providing recommendations pertaining to particular skin care regimens based on the efficacy of a particular therapeutic treatment balanced against any potential conflicts or problems as they relate to the genetic profile of an individual.

FIG. 3 is a flow diagram that illustrates of a non-limiting example of the process for a healthcare provider in interacting with a system according to some embodiments disclosed herein.

FIG. 4 illustrates a non-limiting exemplification of data stores accessed to generate a recommendation for skin care regimen.

FIG. 5 is a flow diagram illustrating a non-limiting example of computer system that can perform the methods of the application.

FIG. 6 is a flow diagram that illustrates a non-limiting example of portals for interacting with the system for an individual (or an associated provider).

### DETAILED DESCRIPTION

Before describing various embodiments of the present disclosure in detail, it is to be understood that this disclosure is not limited to the specific parameters and description of the particularly exemplified systems, methods, and/or products that may vary from one embodiment to the next. Thus, while certain embodiments of the present disclosure will be described in detail, with reference to specific features (e.g., configurations, parameters, properties, steps, components, ingredients, members, elements, parts, and/or portions, etc.), the descriptions are illustrative and are not to be construed as limiting the scope of the present disclosure and/or the claimed invention. In addition, the terminology used herein is for the purpose of describing the embodiments, and is not necessarily intended to limit the scope of the present disclosure and/or the claimed invention.

Personalized human health care products and services that enable individuals to more actively manage their health based at least upon their genetic profiles have been increasingly heralded following the publication of a draft human genome sequence in June 2000. To date, however, the commercial availability of personalized genetic profile products and services has been very limited. The present disclosure generally relates to methods, systems, kits, and related materials for assessing skin condition (e.g., skin health) of an individual based at least in part upon the individual's genetic profile. In particular, the disclosure provides systems, methods, kits, and materials useful for determining the likelihood of an individual to exhibit a plurality of skin phenotypic attributes. Some embodiments disclosed herein relate to methods for identifying a skin care regimen for an individual. Some embodiments provide methods for selecting a personalized skin care regiment for an individual. Further provided, in various embodiments of the application, are kits for assessing skin health and computer systems for displaying a personalized genetics profile.

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented herein. It will be readily understood that the aspects of the present application, as generally described herein, and illustrated in the Figures, can be arranged, substituted, combined, and designed in a wide variety of different configurations, all of which are explicitly contemplated and make part of this application.

Additionally, it is to be understood that the application is not limited to the particular methodologies, protocols, assays, and reagents described herein, as these may vary. It is also to be understood that the terminology used herein is intended to describe particular embodiments of the present application, and is in no way intended to limit the scope of the present application as set forth in the appended claims.

### A. Abbreviated List of Defined Terms

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. Thus, for example, a reference to “a genetic variation” includes a single genetic variation as well as a plurality of such genetic variations, and a reference to “a skin care regimen” is a reference to one or more skin therapeutic and/or dietary regimens and equivalents thereof known to those skilled in the art, and so forth. “A and/or B” is used herein to include all of the following alternatives: “A”, “B”, “A or B”, and “A and B”.

“About” means either within plus or minus 10% of the provided value, or rounded to the nearest significant figure, in all cases inclusive of the provided value. Where ranges are provided, they are inclusive of the boundary values.

The terms “allelic variant” or “allele” are used without distinction in the present application and refer to the different nucleotide sequence variants found at different polymorphic regions. The nucleotide sequence variants may be a single or multiple base changes, including without limitation insertions, deletions, or substitutions, or may be a variable number of sequence repeats.

The term “allelic pattern” refers to the identity of an allele or alleles at one or more polymorphic regions. For example, an allelic pattern may consist of a single allele at a polymorphic site, as for BCMO1 (rs12934922) allele 1. Alternatively, in some embodiments, an allelic pattern may consist of either a homozygous or heterozygous state at a single polymorphic site. For example, BCMO1 (rs1801282) allele 2.2 is an allelic pattern in which there are two copies of the second allele and corresponds to the homozygous BCMO1 (rs1801282) allele 2 state. In addition or alternatively, an allelic pattern may consist of the identity of alleles at more than one polymorphic site.

The expression “amplification” or “amplify” includes methods such as PCR, ligation amplification (or ligase chain reaction, LCR) and amplification methods. These methods are known and widely practiced in the art. In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

Reagents and hardware for conducting PCR are commercially available. Primers useful to amplify sequences from a particular gene region are preferably complementary to, and hybridize specifically to sequences in the target region or in its flanking regions. Nucleic acid sequences generated by amplification may be sequenced directly. Alternatively the amplified sequence(s) may be cloned prior to sequence analysis. A method for the direct cloning and sequence analysis of enzymatically amplified genomic segments is known in the art.

The terms “biomarker” and “genetic marker”, as used interchangeably herein, refers to a sequence consisting of an identifiable nucleic acid sequence that is variable (polymorphic) for different individuals within a population. In general, biomarkers may facilitate the study of inheritance of a trait or a gene. In some embodiments, such biomarkers are used in mapping the order of genes along chromosomes and in following the inheritance of particular genes; genes closely linked to the marker or in linkage disequilibrium (LD) with the marker will generally be inherited with it. In some embodiments disclosed herein, the term biomarker refers generally to “one or more genetic variations,” as that term is defined herein, of which two preferred types of biomarkers are microsatellites and single nucleotide polymorphisms (SNPs), which are commonly used in genetic analysis. Detailed information for individual biomarkers described herein as well as their association with relevant skin-health related conditions can be readily accessible online at, for example, Pharmacogenomics Knowledgebase (PharmGKB) which is publically available on the worldwide web at www.pharmgkb.org/index.jsp.

As used throughout this application the words “can” and “may” are used in a permissive sense (i.e., meaning having the potential to), rather than the mandatory sense (i.e., meaning must). Additionally, the terms “including,” “having,” “involving,” “containing,” “characterized by,” as well as variants thereof (e.g., “includes,” “has,” “involves,” “contains,” etc.), and similar terms as used herein, including within the claims, shall be inclusive and/or open-ended, shall have the same meaning as the word “comprising” and variants thereof (e.g., “comprise” and “comprises”), and do not exclude additional un-recited elements or method steps, illustratively.

The terms “control” or “control sample” refer to any sample appropriate to the detection technique being employed. The control sample may contain the products of the genetic variation detection technique employed or the material to be tested. Further, the controls may be positive or negative controls. By way of example, where the genetic variation detection technique is PCR amplification, followed by size fractionation, the control sample may comprise nucleic acid fragments of an appropriate size. Likewise, where the genetic variation detection technique involves detection of a mutated protein, the control sample may comprise a sample of a mutant protein. However, in some embodiments, it is preferred that the control sample comprises the material to be tested. For example, the controls may be a sample of genomic DNA or a cloned portion thereof containing one or more genes. In some embodiments, where the sample to be tested is genomic DNA, the control sample is preferably a highly purified sample of genomic DNA.

Various aspects of the present disclosure, including devices, systems, and methods may be illustrated with reference to one or more embodiments or implementations, which are exemplary in nature. As used herein, the terms “embodiment” and “implementation” mean “serving as an example, instance, or illustration,” and should not necessarily be construed as preferred or advantageous over other embodiments disclosed herein. In addition, reference to an “implementation” of the present disclosure or invention includes a specific reference to one or more embodiments thereof, and vice versa, and is intended to provide illustrative examples without limiting the scope of the invention, which is indicated by the appended claims rather than by the following description.

The term “gene” is used broadly to refer to any segment of nucleic acid molecule that encodes a protein or that can be transcribed into a functional RNA. Genes may include sequences that are transcribed but are not part of a final, mature, and/or functional RNA transcript, and genes that encode proteins may further comprise sequences that are transcribed but not translated, for example, 5′ untranslated regions, 3′ untranslated regions, introns, etc. Further, genes may optionally further comprise regulatory sequences required for their expression, and such sequences may be, for example, sequences that are not transcribed or translated. Genes can be obtained from a variety of sources, including cloning from a source of interest or synthesizing from known or predicted sequence information, and may include sequences designed to have desired parameters.

The term “genotype” as used herein refers to the genetic information an individual carries at one or more positions in the genome. In some embodiments, a genotype may represent a single locus and in others it may represent a genome-wide set of loci. In another embodiment, the genotype can reflect the sequence of a portion of a chromosome, an entire chromosome, a portion of the genome, and the entire genome. As such, the term “genotype” refers to the specific allelic composition of an entire cell or a certain gene. Genotype can be indirectly characterized using markers or directly characterized by nucleic acid sequencing. Suitable markers include a phenotypic character, a metabolic profile, a genetic marker, or some other type of marker. A genotype may constitute an allele for at least one genetic marker locus or a haplotype for at least one haplotype window. As used herein, “phenotype” means the detectable characteristics of a cell or organism which are typically a manifestation of gene expression.

As used herein, the terms “genotyping,” “haplotyping,” and “DNA typing” are used interchangeably to refer to the determination of the alleles of a selected chromosome or a portion of a chromosome of an individual. As such, “genotyping” an individual (or DNA sample) for a polymorphic allele of a gene (s) involves detecting which allelic or polymorphic form (s) of the gene (s) are present in an individual (or a sample derived therefrom). As is well known in the art, an individual may be heterozygous or homozygous for a particular allele.

The term “one or more genetic variations,” as used herein, refers to any variation in nucleic acid sequence or protein sequence in one or more cells of an individual as compared to the corresponding wild-type genes or proteins. For the purpose of the present application, one or more genetic variations include, but are not limited to, genetic mutations, gene amplifications, splicing variants, insertions, deletions, insertions/deletions (i.e., InDel mutation), gene rearrangements, single-nucleotide polymorphisms (SNPs), single-nucleotide variations (SNVs), and/or aberrant RNA/protein expression. In the present application, nucleotide substitutions are indicated by (⁻⁻>). For example, the genetic variation rs374588791 (7264G⁻⁻>T) refers to a G-to-T nucleotide substitution at position 7264. All nucleotide positions are typically given on the positive chromosomal strand, which is not necessarily the plus strand of the gene.

The term “genetic profile”, as used herein, refers to one or a set of signature genetic changes (e.g., polymorphisms or genetic variations). As such, a “genetic profile” as used herein comprises information regarding the presence or absence of one or more genetic variations in an individual. A genetic profile can consist of a variety of genetic variations, including genetic mutations, gene amplifications, splicing variants, deletions, insertions/deletions (i.e., InDel mutation), gene rearrangements, SNPs, insertions, and aberrant RNA/protein expression, microsatellites, and minisatellites. A “haplotype” is one or a set of signature genetic changes (i.e., a genetic profile) that includes markers that are normally grouped closely together on the DNA strand, and are usually inherited as a group.

The terms “healthcare provider” or “healthcare professional”, as used interchangeably herein, refers to any person or entity that provides health care services to the individual. Such people or entities may include, but are not limited to, any of the following: caregivers, doctors, pharmacists, hospital employees, laboratory workers, physicians, nurses, aides, emergency medical technicians (EMTs), insurance companies, non-governmental organizations (NGOs), health maintenance organizations (HMOs) and pharmaceutical companies.

As used herein, the terms “increased”, “higher”, “greater”, “faster” or similar terms in association with the ability of an individual with a certain genotype to respond to a treatment or a therapeutic regimen refers to or means having average or above average activity (the activity associated with such terms, not meant to be positive or negative) to such treatments, (e.g., faster metabolism, increased efficacy or apposingly, increased vulnerability to side effects, or increased tolerance to treatments) in comparison to similarly situated individuals with genotype(s). Alternatively, the terms “decreased”, “lower”, “reduced” or similar terms in association with the ability of individuals with a certain genotype to respond to a treatment or a therapeutic regimen means having less or reduced response to such treatments or therapeutic regimens, increased vulnerability to side effects, or reduced tolerance to treatment or therapeutic regimen in comparison to similarly situated individuals with different genotype(s).

An “instructional material”, as used herein, refers to a publication, a recording, a diagram, or any other medium of expression which can be used to communicate how to use a kit described herein, numerical values for weighting the significance of various polymorphisms and genetic variations that are detectable using the kit. The instructional material of the kit of the present application can, for example, be affixed to a container which contains a kit described herein or be shipped together with a container which contains the kit. In addition or alternatively, the instructional material can be shipped separately from the container with the intention that the instructional material and the kit be used cooperatively by the recipient.

The term “isolated” as used herein with respect to nucleic acids, such as DNA or RNA, refers to molecules separated from other DNAs or RNAs, respectively, which are present in the natural source of the macromolecule. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments that are not naturally occurring as fragments and would not be found in the natural state. The term “isolated” is also used herein to refer to polypeptides that are isolated from other cellular proteins and is meant to encompass both purified and recombinant polypeptides.

As used herein, the term “label” intends a directly or indirectly detectable compound or composition that is conjugated directly or indirectly to the composition to be detected, e.g., polynucleotide so as to generate a “labeled” composition. The term also includes sequences conjugated to the polynucleotide that will provide a signal upon expression of the inserted sequences, such as green fluorescent protein (GFP) and the like. The label may be detectable by itself (e.g. radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component.

Examples of luminescent labels that produce signals include, but are not limited to bioluminescence and chemiluminescence. Detectable luminescence response generally comprises a change in, or an occurrence of, a luminescence signal. Suitable methods and luminophores for luminescently labeling assay components are known in the art and described for example in Haugland, Richard P. (1996) Handbook of Fluorescent Probes and Research Chemicals (6 ed.). Examples of luminescent probes include, but are not limited to, aequorin and luciferases.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the lain Johnson and Michelle T. Z. Spence. (Molecular Probes Handbook, A Guide to Fluorescent Probes and Labeling Technologies (Invitrogen Corp, 11th ed.). (2010).

In another aspect, the fluorescent label is functionalized to facilitate covalent attachment to a cellular component present in or on the surface of the cell or tissue such as a cell surface marker. Suitable functional groups, including, but not are limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

The phrase “likelihood to exhibit”, as used herein, means that the individual is more likely than not to exhibit at least one of the described skin phenotypic attributes, identified above, as compared to a similarly situated individual (i.e. control or reference). Any skin care regimen can be used as preselected, directed, or indicated. Certain regimens may show greater efficacy or reduced side effects with certain individuals based on their genetic profile, and thus may be preferred, or alternatively, show reduced efficacy or greater side effects, or have other limitations which may then be preselected with precaution, certain limitations or removed from use.

As used herein, an “Enhanced”, “Diminished”, or “Average” likelihood of exhibiting one or more phenotypic attributes, or a “relative likelihood,” is with respect to the general population in a particular geographical area or areas, or with respect to a defined subpopulation thereof, for example, but not limited to, a particular gender, age grouping, or ethnicity, or some other identifying feature.

The term “mismatches” refers to hybridized nucleic acid duplexes that are not 100% homologous. The lack of total homology may be due to deletions, insertions, inversions, substitutions or frameshift mutations.

As used herein, the term “nucleic acid” refers to polynucleotides such as deoxyribonucleic acid (DNA), and, where appropriate, ribonucleic acid (RNA). The term should also be understood to include, as equivalents, derivatives, variants and analogs of either RNA or DNA made from nucleotide analogs, and, as applicable to the embodiment being described, single (sense or antisense) and double-stranded polynucleotides. Deoxyribonucleotides include deoxyadenosine, deoxycytidine, deoxyguanosine, and deoxythymidine. For purposes of clarity, when referring herein to a nucleotide of a nucleic acid, which can be DNA or RNA, the terms “adenosine”, “cytidine”, “guanosine”, and “thymidine” are used. It is understood that if the nucleic acid is RNA, a nucleotide having a uracil base is uridine.

The terms “oligonucleotide” or “polynucleotide”, or “portion,” or “segment” thereof refer to a stretch of polynucleotide residues which is long enough to use in PCR or various hybridization procedures to identify or amplify identical or related parts of mRNA or DNA molecules. The polynucleotide compositions of this application include RNA, cDNA, genomic DNA, synthetic forms, and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

The term “patient” generally refers to any animal, for example a mammal, under the care of a physician, as that term is defined herein, with particular reference to humans under the care of a dermatologist, primary care physician or other relevant medical professional. For the purpose of the present application, a “patient” may be interchangeable with an “individual.” In some embodiments, the individual is a human patient. In alternative embodiments, the patient can be a non-human animal or other organism.

The term “physician” as used herein generally refers to a medical doctor, particularly a dermatologist or primary care physician. This term may, when contextually appropriate, include any medical professional, including any licensed medical professional or other healthcare provider, such as a physician's assistant, a nurse, a genetics counselor, a veterinarian (such as, for example, when the patient is a non-human animal), etc.

The term “polymorphism” refers to the occurrence of genetic variations in the nucleotide sequence of nucleic acids or in the amino acid sequence of polypeptides that account for alternative DNA sequences and/or alleles among individuals in a population. When used in reference to a nucleic acid sequence, the term “polymorphic site” refers to a genetic locus wherein one or more particular sequence variations occur. A polymorphic site can be one or more base pairs. For example, a “single nucleotide polymorphism (SNP)” is a polymorphism that occurs at a single nucleotide. A portion of a gene of which there are at least two different forms, i.e., two different nucleotide sequences, is referred to as a “polymorphic region of a gene”. A polymorphic region can be a single nucleotide, the identity of which differs in different alleles; in a particular case, when the variation occurs in just one nucleotide (A, C, T or G) it is called a SNP.

A “polymorphic gene” refers to a gene having at least one polymorphic region.

The term “providing” when used in a method step means generally to bring into existence and may include a range of perspective-based actions, including, as non-limiting examples, supplying, making available for use, receiving, and/or accessing. Therefore, the term “providing” should not be read or understood to restrict actions to a single perspective.

The term “regimen”, as used herein, is descriptive of a regulated action plan or set of rules defined for a particular individual. In some embodiments, a skin care regimen for an individual may include a prescribed course of medical treatment, manner of living, exercise, food or diet for the preservation, promotion, and/or restoration of the individual's skin health. In some embodiments, a skin care regimen for an individual may include combination of drugs, their doses and administration techniques along with a schedule for how often the drugs are to be administered. If the individual takes the proper combination of drugs via the proper techniques and at the prescribed schedule, the health care regimen has a higher likelihood of success.

A “response” implies any kind of improvement or positive response either clinical or non-clinical such as, but not limited to, measurable evidence of diminishing disease or disease progression, complete response, partial response, stable disease, increase or elongation of progression free survival, increase or elongation of overall survival, or reduction in toxicity or side effect vulnerability.

The term “skin nutrition” is used herein to include nutrition, such as foods, liquids, and supplements, that have an effect on the appearance of skin. Types of nutrition that may have an affect on skin appearance and health are known and may include, but are not limited to, Vitamin A, Vitamin B2, Vitamin B6, Vitamin B12, Vitamin B3, Vitamin C, Vitamin D, Vitamin E, Omega 3 fatty acid, omega-6 fatty acid, and/or combinations thereof.

While the detailed description is separated into sections, the section headers and contents within each section are not intended to be self-contained descriptions and embodiments. Rather, the contents of each section within the detailed description are intended to be read and understood as a collective whole where elements of one section may pertain to and/or inform other sections. Accordingly, embodiments specifically disclosed within one section may also relate to and/or serve as additional and/or alternative embodiments in another section having the same and/or similar systems, modules, devices, methods, and/or terminology.

The embodiments disclosed herein will now be described by reference to some more detailed embodiments, with occasional reference to any applicable accompanying drawings. These embodiments may, however, be embodied in different forms and should not be construed as limited to the embodiments set forth herein. Rather, these embodiments are provided so that this disclosure will be thorough and complete, and will fully convey the scope of the embodiments to those skilled in the art.

### B. Non-Limiting Embodiments of the Application

In some embodiments, the present application relates to systems and methods for determining the likelihood of an individual to exhibit one or more skin phenotypic attributes. Such systems and methods, in some embodiments, include (a) acquiring knowledge of the occurrence of one or more genetic variations associated with each member of a first set and a second set of preselected biomarkers in the individual, wherein each member of the first set of biomarkers is genetically associated with one or more skin nutritional conditions and each member of the second set of biomarkers is genetically associated with one or more skin phenotypic attributes; (b) generating a personalized biomarker profile for the individual from the acquired knowledge; and (c) determining the status of the individual's nutritional skin health and the likelihood of the individual to exhibit the one or more skin phenotypic attributes based at least in part upon the personalized biomarker profile. Some embodiments include (a) providing a biological sample; (b) determining at least a portion of a genotype from the biological sample by identifying one or more genetic variations associated with a plurality of skin phenotypic attributes, the plurality of skin phenotypic attributes comprising one or more skin nutritional conditions and one or more skin health characteristics, and the one or more genetic variations comprising a first set of preselected genetic variations and a second set of preselected genetic variations, each member of the first set of preselected genetic variations being genetically associated with one or more skin nutritional conditions and each member of the second set of preselected genetic variations being genetically associated with one or more skin health characteristics; (c) generating a personalized biomarker profile for the individual based on the identified one or more genetic variations; and (d) determining the likelihood of the individual to exhibit the plurality of skin phenotypic attributes based at least in part upon the personalized biomarker profile.

Thus, in some embodiments, and as used throughout the present application, the act of acquiring knowledge may encompass a variety of steps or actions. For example, acquiring knowledge may include determining the presence of one or more genetic variations within a sample by sequencing nucleic acid isolated from the sample. It may further include performing a proteomic analysis, biochemical assay, or even performing bioinformatics on one or more data associated with the occurrence of one or more genetic variations associated with a sample, particularly one or more genetic variations associated with the aforementioned first and second sets of preselected biomarkers in an individual. Accordingly, acts such as determining at least a portion of a genotype from a biological sample falls within the understanding of acquiring knowledge, as used herein, regardless of the means by which the genotype is determined (e.g., an analytical assay performed on nucleic acid or by an antibody-based assay performed on a protein or peptide fragment).

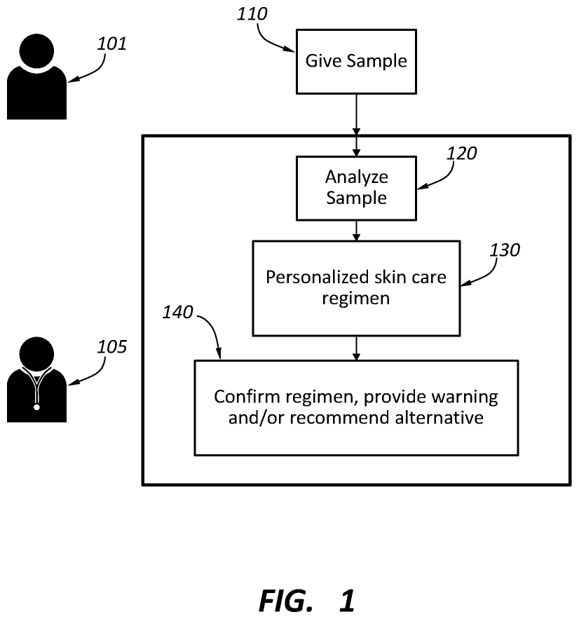

In some embodiments, as illustrated in FIG. 1, the systems and methods disclosed herein comprise an individual 101 giving and/or providing a sample 110. In some embodiments, the individual may be a patient, an individual diagnosed with a particular skin health related condition, or an individual desirous for additional information about their skin health or likelihood of exhibiting a skin phenotypic attribute (or a plurality of skin phenotypic attributes). The sample may be analyzed 120, which may in some embodiments include acquiring knowledge of the occurrence of one or more genetic variations and/or determining at least a portion of a genotype from the biological sample. The latter may be accomplished, in some embodiments, by identifying one or more genetic variations associated with one or more skin phenotypic attributes, which may be done in a nucleic-acid-dependent fashion (e.g., sequencing, PCR amplification, DNA probe, or any other methods recited herein).

The act 120 of analyzing the sample may additionally include generating a personalized genetic profile for the individual 101, which may in turn be used as at least one element in determining the likelihood of individual 101 to exhibit one or a plurality of skin phenotypic attributes. In particular, the present application provides a grading/weighting method and system for determining an individual's likelihood to exhibit one or a plurality of skin phenotypic attributes. In some embodiments, this includes identifying, characterizing, and/or applying one or more criteria to the biomarkers that act to score/weigh biomarkers to increase, decrease, or neutrally affect the impact or likelihood that the biomarkers are informative with respect to their ability to bring about a given phenotypic attribute in an individual (e.g., skin nutritional conditions or skin health characteristics). In some embodiments, the criteria include any criterion or combination of criteria: family history, general medical physiological measures, cholesterol levels, blood pressure, heart rate, growth hormone levels, insulin sensitivity, obesity, body weight, triglyceride levels, red blood cells, bone density, CD scan results, mRNA expression profiles, methylation profiles, protein expression profiles, enzyme activity, nucleotide sequence homology, expression level, enzyme activity, relative synteny among the preselected biomarkers, family history, ontological relevance, quality of supporting research, and degree of phenotypic significance. In some embodiments, the foregoing list is, itself, weighted or otherwise hierarchical with respect to the importance of the given criterion to the overall impact of the presence of the genetic variation and its ability to predict the likelihood of a phenotypic attribute presenting in an individual.

For example, the genetic variations may be nucleic acid in nature. In such embodiments, the genetic variations may include SNPs that have been identified in a genome wide association study or otherwise published as part of the results in a scientific article. In instances such as these, the quality of supporting research and/or the degree of phenotypic significance provided in the research may weigh heavily in the calculus for determining the likelihood that a genetic variation is indicative for determining the expression (or likely expression) of a phenotypic attribute. Accordingly, in some embodiments the foregoing two criteria significantly contribute to the score or weighting applied to the genetic variation. In some embodiments, the score/weight associated with a given genetic variation may be a compilation of multiple studies. That is, the assigned score/weight may be the result of an aggregation and/or normalization of multiple individual weights derived from a plurality of studies each have their own identifiable quality of supporting research and/or reported degree of phenotypic significance. In some embodiments, the quality of supporting research and/or the degree of phenotypic significance may be similar or it may vary.

For example, a SNP may be described in three different studies, each study having a quantitatively strong quality of supporting research. Each of the studies may be weighted based alone, or in part, on the quality of supporting research, and in some embodiments, a single weight representing the quality of the supporting research (or any other metric/criteria) may be computed at a computing system or determined by a user (e.g., a physician, a database manager, a scientist, a curator, etc.). In some embodiments, the individual weights associated with each separate study may be aggregated into a single value by adding the values together, or they may be combined by any other means known in the art, such as, for example, by averaging the individual weights.

In some embodiments, to determine the strength of supporting research, some standardized criteria may be used, including, as non-limiting examples, the number of participants in the study, the number and types of positive and negative controls provided in the study, ethnic or gender matching (or other type matching), and/or whether the study was peer reviewed. A strong study may include at least, for example, more than 5,000 participants with more than 5,000 controls. In some embodiments, a strong study may include more than 2,500 participants, more than 2,000 participants, more than 1,500 participants, more than 1,000 participants, more than 750 participants, more than 500 participants, less than 500 participants, less than 400 participants, less than 300 participants, less than 200 participants, less than 100 participants, or ranges falling between any of the foregoing and each having the same or similar number of control participants. The strength of a study may, in some embodiments, be negated or reduced by conflicting data/evidence in the same study or in a separate study.

Additionally, or alternatively, the reported degree of phenotypic significance of a genetic variation may affect the weight of a given study and/or ultimately the overall weight associated with a genetic variation. For example, a finding showing no statistical correlation between a genetic variation and a phenotype will have a low weight or, in some embodiments, a negating weight. On the other hand, a finding showing a high statistical correlation with a low p-value may provide the genetic variation with a strong weight. In some ways, this may be intuitive. That is, a genetic variation that has previously demonstrated a statistically strong correlation with a given phenotypic attribute is more likely to influence a likelihood that a given phenotypic attribute will be exhibited in an individual as opposed to one that has a statistically weak correlation. In some embodiments, the weight associated with the reported degree of phenotypic significance may be threshold based. For example, studies reporting p-values ranging from 0.05>p>0.01 may have a first value and 0.01>p>0.001 may be associated with a second value that is a scalar multiple of the first value, and 0.001>p>0.0001 may be associated with a third value that is a scalar multiple of the second value, and so on. Additionally, or alternatively, the weight associated with the reported degree of phenotypic significance may be directly related to the reported p-value. In some embodiments, the reciprocal of the p-value (which may, in some embodiments, be multiplied by a constant to normalize the values) may be used, or the negative logarithm of the p-value may be used. In any of the foregoing embodiments, the reported p-value is transformed into a weight or value to be applied to the weight associated with a given genetic variation.

The foregoing criteria may be combined or used alone to arrive at a weight associated with the genetic variation. As an exemplary embodiment illustrating the foregoing, a first genetic variation has two studies associated with it and a second genetic variation is associated with three studies. The two studies associated with the first genetic variation have large sample sizes (e.g., greater than 1000 participants), no conflicting data, and strong controls. Each of the major findings (both genome wide association studies) in the two studies were statistically significant, the first with a p<0.05 and the second with p=0.001. On the other hand, the three studies associated with the second genetic variation varied widely in many respects. A first of the three had less than 100 participants with a statistically significant correlation (p<0.0001), which conflicts with the statistically significant correlation found in the second of the three studies (p<0.05). The latter study had over 1000 participants but lacked necessary controls and failed to account for ethnicity and gender. The third study had no statistically significant findings but was otherwise a strong study (e.g., large sample size, good controls, ethnic matching, etc.).

In this non-limiting example, a weight is collectively calculated for each of the two groups of studies. Because the two studies associated with the first genetic variation had no evidence of conflicting data and were, additionally replicated (which may, in some embodiments, be an indicator or a necessary element for assigning a strong weight), a strong weight (e.g., a large number or a persuasive modifier, etc.) is associated with the first genetic variation, which implies that the first genetic variation has a higher likelihood of influencing the phenotypic attribute for which it is associated. On the other hand, the three studies associated with the second genetic variation were contradictory or statistically non-informative. Further, the study with the lowest p-value had a relatively low sample size, whereas the study demonstrating significance with p<0.05 had a large sample size but poor controls. Without considering the contradictory findings, the first and second studies are likely to have moderate to weak weights associated therewith; the third study, while having a good sample size and other similarly good qualities indicative of high strength supporting research, cannot be granted a weight due to its inconclusive statistical findings (in some embodiments, the strength of the supporting research may be evaluated separately from the degree of phenotypic significance; in such cases, a strong weight may be given to this reference for its strength of supporting research but a low weight (or even a null weight) may be given for the degree of phenotypic significance). Thus, in the current embodiment, the remaining two studies could be combined to arrive at the weight. However, these studies were conflicting, and in some embodiments, a conflicting result negates the utility of the genetic variation as a potential indicator of assessing skin health. In other embodiments, the weights may be combined, each having opposite degrees (e.g., one a positive weight, the other a negative weight) and weighted together with the other criteria to arrive at a single weight associated with the genetic variation.

Though the weights in the foregoing example were collectively calculated for each associated genetic variation, weights may, in some embodiments, be individually calculated for each reference and then aggregated to determine the overall weight. The foregoing may be better exemplified in embodiments where the first and second genetic variations are associated with the same phenotypic attribute. In such an instance, the weight of the phenotypic attribute (or the collective weight of each genetic variation associated therewith) may be calculated by, for example, an algorithm that computes the overall likelihood of phenotypic attributes being exhibited in an individual.

In some embodiments, the likelihood is a relative measure. It may in some embodiments be represented as normal, reduced, increased, or high. These relative measures may be chosen by, for example, plotting the weights of all genetic variations (or similar) on a graph and/or histogram and defining quartiles within the plotted weights—each quartile bounded by one or more weights values. The effect of each genetic variation on the phenotypic attribute may determine the relative measure of likelihood.

In some embodiments, the relative measures of likelihood are based on thresholds associated with and/or garnered from associated research. Returning to the exemplary embodiment above, the first genetic variation included two references. The lowest reported p-value (i.e., p<0.05) is used to generate a first threshold. The highest reported p-value (i.e., p=0.001) is used to generate a second threshold, the second threshold being higher than the first threshold. In each instance, the negative logarithm of the p-value is calculated. Thus, −log(0.05)=1.301 and −log(0.0001)=4, making the first, lower threshold 1.301 and the second higher threshold 4. A positive value lower than the first, lower threshold (i.e., between 0 and 1.301) is considered a normal/average likelihood. A value between the first and second threshold (i.e., between 1.301 and 4) is considered an increased likelihood. A value greater than the second, higher threshold (i.e., greater than 4) is considered a high likelihood. In some instances, a genetic variation may actually reduce the likelihood. In such instances, any negative value is indicative of a reduced likelihood.

The aforementioned value that is placed on the threshold-based scale is, in some embodiments, the cumulative weighted value for a given genetic variation. In the foregoing example, the cumulative weighted value may be calculated by aggregating the individual weights associated with each study (in some embodiments, the weights for each genetic variation associated with a given phenotypic attribute are aggregated). In some embodiments, a scalar (constant or variable) may be applied to one or more weights when aggregating. As an elementary example, the two studies in the exemplary embodiment will be given a strong weight for the strength of supporting research. As used herein, a strong weight is equal to 1, a moderate weight is equal to 0.75, a weak weight is equal to 0.5, and a preliminary weight is equal to 0.25. The weights for supporting research can be aggregated with, for example, the −log(p-value) associated with each study. Thus, the combined weight, CW=(1)(−log(0.05))+(1)(−log(0.0001)) or CW=1.301+4=5.301. Based on the thresholds previously set, 5.301 is greater than 4, making the combination of genetic variations highly likely at exhibiting the associated phenotypic attribute.

One having skill in the art will appreciate that if the supported research strengths were reduced, say to 0.75 each, then CW=3.98, which is less than 4, making the likelihood fall from highly likely to an increased likelihood. The foregoing is illustrative of the general principles by which a likelihood may be determined. Weights or measures may be added, removed, or augmented and be within the scope of the disclosure.

One having skill in the art will also appreciate that the foregoing provides an advantage and differentiating factor. Particularly, the number and types of genetic variations utilized in determining the likelihood of an individual to exhibit skin phenotypic attributes is different than all known genetic variations. That is, the number of genetic variations evaluated in determining the likelihood of an individual to exhibit skin phenotypic attributes is, in some embodiments, a subset of the total known genetic variations for skin phenotypic attributes. For example, as illustrated in the foregoing exemplary embodiment, the second genetic variation was excluded altogether from the calculus for determining a likelihood, even though there was a single study characterizing the genetic variation as demonstrating a statistically significant correlation. Additionally, or alternatively, the differential and/or combined weighting of genetic variations or studies associated with genetic variations may temper the perceived importance of one study over another, or may increase the importance or weight attributed thereto. For example, a single strong study/genetic variation amidst a throng of weak studies/genetic variations associated with the same phenotypic attribute may provide a more holistic, unbiased view. Further, not every individual will possess every genetic variation. More realistically, each individual will encode a subset of genetic variations associated with one or more skin phenotypic attributes. The previously disclosed weighting system and methods allows for a personalized and individual insight into the likelihood an individual will exhibit one or more skin phenotypic attributes.

Thus, in one or more embodiments of the present disclosure, the sets of one or more genetic variations comprise unique sets of genetic variations. For example, in one or more embodiments, the first set of preselected genetic variations and the second set of preselected genetic variations, wherein each member of the first set of preselected genetic variations is genetically associated with the one or more skin nutritional conditions and each member of the second set of preselected genetic variations is genetically associated with the one or more skin health characteristics, are unique sets. Additionally, the foregoing unique sets of genetic variations can be differentially and individually applied (together with their weighting) based on the personalized genetic profile of an individual, providing an advantage over similar genetic-based predictive systems, kits, and/or methods.

Similar as to that described above, in some embodiments, the present application comprises an algorithm or system, wherein a skin care regimen or a dietary regimen is assigned to categories such as one of the four categories below.

1. Used as Directed,

2. Preferential Use,

3. May Have Limitations,

4. May Cause Serious Adverse Events.

In some embodiments, the present application comprises an algorithm or system, wherein a likelihood of exhibiting a skin characteristic is assigned to categories such as one of the categories below.

1. Very High Risk,

2. High Risk,

3. Increased Risk/Above Average Risk,

4. Typical/Average Risk/Healthy/Normal,

5. Diminished Risk/Reduced Risk, Below Average Risk.

For example, in some embodiments, each skin characteristic is assigned to the default category, “Typical/Average Risk/Healthy/Normal”, unless it is reassigned to another category based on genetic test result(s). In case the skin characteristic can be reassigned to multiple categories because of results from multiple genetic tests, the category that invokes most precautionary measures (e.g., least positive) will apply to the micro nutrient or skin condition. As defined herein, the term “least positive” refers to the most precautionary category or measure or assessment that can be attributed to an individual based on their potential response to skin care regimens. For example, the assessment for an individual with respect to their response to a particular dietary regimen may be positive or normal with respect to all aspects except, for example, a potential negative adverse reaction to skin inflammation. The potential negative reaction would be the least positive or most precautionary assessment, and would be the recommendation to the patient, e.g., the individual may be at risk for potential negative adverse reactions.

The input of the algorithm typically includes the genotyping results of the tested individual. In some embodiments and similar to what that provided above with respect to weighting genetic variations or other criteria associated therewith and or with one or more phenotypic attributes, the input of the algorithm further includes information relating to one or more criteria upon which the biomarkers within the preselected biomarkers can be selected. Such criteria can include, for example, nucleotide sequence homology, expression level, enzyme activity, relative synteny among the preselected biomarkers, family history, ontological relevance, quality of supporting research, and degree of phenotypic significance. In some embodiments, the input of the algorithm further includes information general medical physiological measures and values general medical physiological measures or values (such as, but not limited to, cholesterol levels, blood pressure, heart rate, growth hormone levels, triglyceride levels, red blood cells, bone density, CD scan results, etc.), mRNA expression profiles, methylation profiles, protein expression profiles, enzyme activity, antibody load, and family history.

The output of the algorithm typically includes the recommendation categories for all tested characteristics, skin care regimens, dietary/nutritional regimens, and a text for each regimen that is not assigned to the “Use as Directed” category. The text includes detailed reasons for the category assignment and, when appropriate, clinical recommendations.

In some embodiments, the output of the algorithm further features skin care regimens, which are selected based, at least in part, on determination of the identity of the polymorphic region or expression level (or both in combination) of the biomarkers described herein.

In various embodiments, the algorithm can include one or more of the following components:
1. A library of candidate recommendation category assignments for all skin characteristic-genotype combinations,
2. A library of texts for all skin characteristic-genotype combinations,
3. Rules for determining the final skin characteristic recommendation categories,
4. Rules for selecting texts for display in the test report, and
5. Rules for assessing the impact of incomplete test results.

Referring back to FIG. 1, in some embodiments, step 130 includes identifying a personalized skin care regimen for individual 101 and/or further confirming, recommending or prescribing such a personalized skin care regimen to individual 101. In some embodiments, step 140 is optional; in some embodiments, it is mandatory. Step 140 includes confirming the regimen, providing a warning, and/or recommending an alternative, which may, in some embodiments, be provided by a physician 105 or other medical professional.

In some embodiments, the methods and systems disclosed herein comprise analyzing an individual's genetic profile which comprises an array of genetic variations. In some embodiments, the methods and systems disclosed herein comprise acquiring knowledge of the occurrence of one or more of such genetic variations to generating a personalized biomarker profile for the individual from the acquired knowledge, determining the status of skin nutritional health of the individual and the likelihood of the individual to exhibit a plurality of skin phenotypic attributes based at least in part on the acquired knowledge, and identifying a skin care regimen appropriate for the individual based at least in part upon the determined status of skin nutritional health and the determined likelihood of the individual to exhibit the one or plurality of skin phenotypic attributes.

In some embodiments, the knowledge (or determination) of the individual's genetic profile is acquired from the occurrence of one or more genetic variations associated with each member of two sets of biomarkers in the individual. The first set of biomarkers can include one or more biomarkers, each of which is genetically associated with one or more skin nutritional conditions. The second set of biomarkers can also include one or more biomarkers, each of which is genetically associated with one or more skin health characteristics.

In some embodiments, each of the preselected first and second set of biomarkers independently include 1, 5, 10, 15, 20, 25, 50, 100, 200, 300, 500, 750, or 800 biomarkers or a number of biomarker that is within a range defined by any two of the aforementioned numbers. In some embodiments, each of the preselected first and second set of biomarkers independently include at least 1, 2, 3, 4, 5, 6, 7, 8, 9, 10, 15, 20, 25, 30, 35, or 40 biomarkers. In some embodiments, the numbers of biomarkers of the preselected first and second set of biomarkers can be the same. In some embodiments, the numbers of biomarkers of the preselected first and second set of biomarkers can be different.

In some embodiments, each of the preselected first and second set of biomarkers independently include biomarkers that map to at least about 2, 5, 10, 25, 30, 35, 40, 100, 200, or 500 discrete loci, or a number of loci that is within a range defined by any two of the aforementioned numbers. In some embodiments, each of the preselected first and second sets of biomarkers independently include genetic markers that map to at least about 5 discrete loci. In some embodiments, each of the preselected first and second set of biomarkers independently include genetic markers that map to at least about 10 discrete loci. In some embodiments, each of the preselected first and second sets of biomarkers independently include genetic markers that map to at least about 15 discrete loci. In some embodiments, each of the preselected first and second sets of biomarkers independently include genetic markers that map to at least about 20 discrete loci.

In some embodiments, the first and/or second sets of biomarkers may further include one or more subsets of biomarkers. In some embodiments, the first and second sets of biomarkers each include a different number of subsets, and in other embodiments, the first and second sets of biomarkers have the same number of subsets. As a non-limiting example, the first and second set may be defined by a single subset, (excluding the empty set); that is, each member of the first subset is in the same subset, and each member of the second subset is in a same subset, albeit different than the first subset of the first set. This is similar to saying the subset of the first set comprises the whole set (of elements in the first set), and similarly, the subset of the second set comprises the whole set (of elements in the second set). Additionally, or alternatively, the second subset may include a plurality of subsets. In some embodiments, the biomarkers associated with the second set of genetic variations genetically associated with one or more skin health characteristics may be divided (evenly or unevenly) into different groups based on any number of partitioning schemes. For example, the genetic variations genetically associated with one or more skin health characteristics may be divided into subsets based upon the type or quality of effects the genetic variation has or is associated with (e.g., a skin photoaging (including skin aging and skin tone) subset, a skin texture and elasticity subset, a skin inflammation, and allergy risk subset, a skin moisture factor subset, a skin oxidation protection subset, and a skin glycation subset). In some embodiments, each biomarker is in its own subset. Any subsets may be combined (e.g., back to the whole set) or divided and may be organized in any other manner (e.g., alphabetically, numerically, randomly, ordered, etc.).

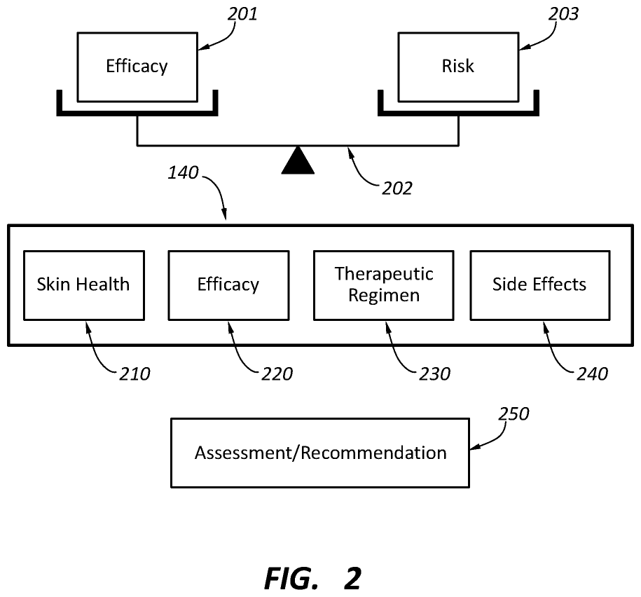

FIG. 2 can be identified as a method and system for genetically evaluating the efficacy 201 of a particular skin therapeutic regimen for a skin health related condition for an individual balanced 202 against any risks 203 associated with the administration of such skin therapeutic regimen. Once a likelihood for exhibiting a particular skin condition is identified, and preferably confirmed 210, the efficacy of a skin therapeutic regimen 220 with respect to the particular individual and the skin condition is balanced against the pharmacokinetics of the skin therapeutic regimen 230 and further weighted by any potential side effects 240 that the individual or the therapeutics may be prone to. A likely or potential skin condition can be assessed by genotyping the individual to determine if they are genetically predisposed to such a skin condition or may be assessed by traditional means of diagnosing such a skin condition. In many cases, the pharmacokinetics of the skin therapeutic regimen will affect the efficacy of the regimen, e.g., tolerance or metabolism of the regimen will affect the skin condition and the individual, and also the side effects or any adverse effects that may arise due to the therapeutic regimen lingering or affecting non-desired pathways. A recommendation or assessment 250 is made based on the weighting of these factors.

### C. Skin Health Characteristics

Methods, systems, kits of the present application rely at least in part upon the finding that there is an association between the patterns of genetic variations of certain metabolic genes and the likelihood of an individual to exhibit one or more skin health attributes, and/or the susceptibility of the individual to particular diets and/or exercise regimens. That is, there is an association between the genetic profile of metabolic genes and skin phenotypes as well as between skin health-related therapeutic outcomes. It has been well documented that particular genes impact various pathways influencing skin health and have been associated with elevated risk or diminished risk for skin disorders and conditions and for their ability to differentiate an individual's response to skin care interventions. For the purposes of this application, such genes will be referred to as “metabolic genes” or “skin-health related genes”.

In some embodiments, the present application provides methods, systems, kits to determine an individual's genetic profile, which include acquiring knowledge of the occurrence of one or more genetic variations associated with preselected biomarkers that are mapped within one or more skin-health related genes, thereby generating a personalized biomarker profile for the individual. The results of such genotyping may be used to determine the likelihood of an individual to exhibit one or more (or a plurality of) skin phenotypic attributes, the status of the individual's nutritional skin health, and/or the individual's likely responsiveness to skin care therapeutic/dietary regimens. Generating a personalized biomarker profile may be used for pairing the individual with a therapeutic, nutritional, or lifestyle alteration, or a combination thereof and/or may be used to devise a strategy to achieve and/or sustain improvements in skin health. Thus, according to at least some embodiments of the application, polymorphism genotyping results may be used to determine the genetic influence on 1) the likelihood of an individual to exhibit one or more (or a plurality of) skin phenotypic attributes, and 2) responsiveness to skin care therapeutic/dietary regimens for skin health improvement.

Collectively, the determination of an individual's biomarker profile for one or more skin-health related genes allows interpretations that provide actionable guidelines for selecting an appropriate therapeutic/dietary regimen or lifestyle recommendation for the individual. By identifying relevant genetic variations, biomarkers, and genotype pattern results, the methods, systems, kits disclosed herein can assess risk for likely outcomes of particular diet types and skin care regimens, and provide the individual with guidance on the appropriate choice of nutrition and lifestyle interventions that match their personal genetic makeup.

Accordingly, in some embodiments, the present application is directed to method, kits and systems for analyzing an array of biomarkers and metabolic genes associated with skin health comprising genotyping genetic variations in an individual to determine a list of actionable items for improvement of the individual's skin condition that includes guidance on a specific diet type that optimizes skin health as well as guidance on skin care routines.

### D. Skin Health-Related Biomarkers and Genes

Many biomarkers have been reported to impact various pathways that influence skin characteristics, many of which have been subsequently demonstrated to be genetically associated with several skin-health related genes. In various embodiments, the methods and systems disclosed herein involve acquiring knowledge of the occurrence of one or more genetic variations associated with biomarkers that are mapped within one or more of skin-health related genes which influence the individual's likelihood of exhibiting one or more of skin phenotypic attributes. In some embodiments, the one or more of skin phenotypic attributes can be: photo aging, wrinkle, freckle, lentigines, ephelids, tanning, stretch marks, cellulite, collagen, skin integrity, icthyosis, skin hydration, eczema, atopic dermatitis, psoriasis, contact dermatitis, rosacea, oxidation response, skin glycation, hyperpigmentation, skin allergies, hyperkeratosis, or a combination thereof.

In some embodiments of the methods and systems disclosed herein, the second set of preselected biomarkers includes biomarkers known to be genetically associated with one or more skin-health characteristics which can be selected from “skin photoaging (including skin aging and skin tone),” “skin texture and elasticity,” “skin moisture factor,” “skin inflammation and allergy risk,” “skin glycation,” “skin oxidation protection,” and/or combinations thereof. Listed below are non-limiting examples of genes that have been shown to be genetically associated with one or more of the above skin-health characteristics. It is to be understood that this list is not exhaustive, but representative of possible genes for analysis.

Detailed information for individual biomarkers, genes, genetic variations listed below as well as their association with relevant skin-health related conditions can be readily accessible online at, for example, Gene Ontology Consortium (GO), KEGG Pathway Database, and Pharmacogenomics Knowledgebase (PharmGKB), all of which are publically available on the World Wide Web at “geneontology.org/”, “www.genome.jp/kegg/”, and “www.pharmgkb.org/index.jsp,” respectively. For example, the PharmGKB database, which was established in 2000, is a publicly available Internet research tool developed to collect, curate and disseminate knowledge about the impact of human genetic variation on responses to therapeutic regimens/treatments through a wide ranges of activities including, inter alia, (1) annotating human genetic variants and “gene-treatment-disease” relationships via literature reviews; (2) summarizing important pharmacogenomic genes, associations between genetic variants and drugs, and drug pathways; (3) displaying all information on the website and providing comprehensive downloads. Among other things, numerous genetic variations (e.g., polymorphisms) associated with specific skin disorders and conditions, their gene sequence information, and corresponding protein products are catalogued in a searchable format. The relevant treatments previously reported to be associated with each of the catalogued genetic variations are also readily identified, validated, annotated, and catalogued in a searchable format. In addition, the PharmGKB database encompasses clinical information including dosing guidelines and drug labels, potentially clinically actionable gene-treatment associations and genotype-phenotype relationships.

The data generated from these analyses can be analyzed using publicly available databases including, but not limited to, the USDA National Nutrient Database for Standard Reference (“ndb.nal.usda.gov//”), the PubMed database (“www.ncbi.nlm.nih.gov/pubmed”), the GWAS catalog from the NHGRI-EBI (“www.ebi.ac.uk/gwas/home”), the ExAc database from the Broad Institute (“exac.broadinstitute.org”), the SNPedia database (“www.snpedia.com/index.php/Rs12272004”), the RegulomeDB database (“regulomedb.org/GWAS/index.html”), the ExPASy database (“www.expasy.org”), the HGNC database (“www.genenames.org”), the dbSNP database (“www.ncbi.nlm.nih.gov/snp”), the ClinVar database (“www.ncbi.nlm.nih.gov/clinvar/”), the OMIM database (“www.ncbi.nlm.nih.gov/omim”), the PheGenI database (“www.ncbi.nlm.nih.gov/gap/phegeni”), and the HapMap database (“hapmap.ncbi.nlm.nih.gov/cgi-perl/gbrowse/hapmap28_B36/”).

Thus, those of skill in the art could readily access sequence information at the nucleotide and protein level that corresponds to each of the genetic variations, biomarkers, and genes described herein in the specification and/or recited in the claims.

Skin Photoaging (Including Skin Aging and Skin Tone)

The term “skin aging” is used herein to include all aspects of the process by which skin changes over the lifetime of an individual, including but not limited to photoaging, wrinkles, freckles (including lentigines and ephielides), the thinning of the outer skin layer or epidermis, changes—most typically a decrease—in the number of pigment-containing cells, the appearance of large pigmented areas such as age spots, liver spot (lentigos), increased bleeding or bruising, elastosis, solar elastosis, decreased oil production, dryness, itching, as well as appearance changes such as growths like skin tags, warts, rough patches (keratoses), and other blemishes, and/or a thinner, paler, clear, or translucent appearance.

The term “skin tone” is used herein to include the coloration of skin, the complexion of skin, the evenness of coloration of skin across an area, such as the face, any discoloration of skin across an area, such as the face, blemishes such as skin pigmentations, freckles, age spots, acne marks, dark areas, melasma, and changes to the coloration and appearance of an area of skin in response to environmental or other factors such as exposure to sun or wind, the undertone of the skin.

In some embodiments, the preselected biomarkers genetically associated with skin aging and skin tone include biomarkers that are mapped within one or more genes selected from: MCIR, TYR, SLC45A2 (MATP), SLC24A5, ASIP Region, HERC2, IRF4, EXOC2, STXBP5L, 6p25.3 Region, MMP1, NCOA6, and/or combinations thereof.

In some embodiments, the preselected biomarkers genetically associated with skin aging and skin tone include biomarkers that are mapped within the MCIR gene. In the some embodiments, the biomarkers mapped within the MCIR gene include: rs1805005, rs2228479, rs885479, rs1805007, rs1805008, rs1805009, rs11547464, rs1110400, rs1805006, and/or combinations thereof.

In some embodiments, the preselected biomarkers genetically associated with skin aging and skin tone include biomarkers that are mapped within the TYR gene. In the some embodiments, the biomarkers mapped within the TYR gene include: rs1393350, rs1126809, rs1042602, and/or combinations thereof.

In some embodiments, the preselected biomarkers genetically associated with skin aging and skin tone include biomarkers that are mapped within the SLC45A2 (MATP) gene. In the some embodiments, the biomarkers mapped within the SLC45A2 gene include: rs16891982, rs26722, and/or combinations thereof.

In some embodiments, the preselected biomarkers genetically associated with photoaging (including skin aging and skin tone) include biomarkers that are mapped within the SLC24A5 gene. In the some embodiments, the biomarkers mapped within the LC24A5 gene include: rs1426654, rs2555364, and/or combinations thereof.

In some embodiments, the preselected biomarkers genetically associated with skin aging and skin tone include biomarkers that are mapped within the ASIP Region. In some embodiments, the biomarkers mapped within the ASIP Region include: rs1015362, rs4911414, and/or combinations thereof.

In some embodiments, the preselected biomarkers genetically associated with skin aging and skin tone include biomarkers that are mapped within the HERC2 gene. In the some embodiments, the biomarkers mapped within the HERC2 gene can include rs12913832.

In some embodiments, the preselected biomarkers genetically associated with photoaging (including skin aging and skin tone) include biomarkers that are mapped within the IRF4 gene. In the some embodiments, the biomarkers mapped within the IRF4 gene can include rs12203592.

In some embodiments, the preselected biomarkers genetically associated with skin aging and skin tone include biomarkers that are mapped within the EXOC2 (SEC5L1) gene. In the some embodiments, the biomarkers mapped within the EXOC2 gene can include rs12210050.

In some embodiments, the preselected biomarkers genetically associated with skin aging and skin tone include biomarkers that are mapped within the STXBP5L gene. In the some embodiments, the biomarkers mapped within the STXBP5L gene can include rs322458.

In some embodiments, the preselected biomarkers genetically associated with skin aging and skin tone include biomarkers that are mapped within the 6p25.3 Region (that is, the intergenic between EXOC2 and IRF4). In the some embodiments, the biomarkers mapped within the 6p25.3 Region can include rs1540771.

In some embodiments, the preselected biomarkers genetically associated with skin photoaging (including skin aging and skin tone) include biomarkers that are mapped within the MIP gene. In the some embodiments, the biomarkers mapped within the MIP1 gene can include rs1799750.

In some embodiments, the preselected biomarkers genetically associated with skin photoaging (including skin aging and skin tone) include biomarkers that are mapped within the NCOA6 gene. In the some embodiments, the biomarkers mapped within the NCOA6 gene can include rs4911442.

### Skin Texture and Elasticity

The term “skin texture” is used herein to refer to the feel, appearance, and consistency of the skin, which can be measured by factors such as smoothness, firmness, stretch marks, cellulite, collagen, and roughness of the skin. The term “skin elasticity” is used herein to refer to the ability of an area of skin to resume its normal shape after being stretched or compressed.

In some embodiments, the preselected biomarkers genetically associated with skin texture and skin elasticity include biomarkers that are mapped within one or more genes selected from the following genes ACE, HIF1A, ELN, SRPX HMCN1, TMEM18, MTHFR, or a combination thereof.

In some embodiments, the preselected biomarkers that are genetically associated with skin texture and skin elasticity include biomarkers are mapped within an ACE gene. In the some embodiments, the biomarkers mapped within an ACE gene can include rs1799752, rs4646994, and/or combinations thereof.

In some embodiments, the preselected biomarkers genetically associated with skin texture and skin elasticity include biomarkers that are mapped within the HIF1A gene. In the some embodiments, the biomarkers mapped within the HIF1A gene can include rs11549465.

In some embodiments, the preselected biomarkers genetically associated with skin texture and skin elasticity include biomarkers that are mapped within the ELN gene. In the some embodiments, the biomarkers mapped within the ELN gene can include rs7787362.

In some embodiments, the preselected biomarkers genetically associated with skin texture and skin elasticity include biomarkers that are mapped within the SRPX gene. In the some embodiments, the biomarkers mapped within the SRPXgene can include rs35318931.

In some embodiments, the preselected biomarkers genetically associated with skin texture and skin elasticity include biomarkers that are mapped within the HMCN1 gene. In the some embodiments, the biomarkers mapped within the HMCN1 gene can include rs10798036.

In some embodiments, the preselected biomarkers genetically associated with skin texture and skin elasticity include biomarkers that are mapped within the TMEM18 gene. In the some embodiments, the biomarkers mapped within the TMEM18 gene can include rs7594220.

In some embodiments, the preselected biomarkers genetically associated with skin texture and skin elasticity include biomarkers that are mapped within the MTHFR gene. In the some embodiments, the biomarkers mapped within the MTHFR gene can include rs1801133 and/or rs1801131.

### Skin Moisture Factor

The term “skin moisture factor” is used herein to include the hydration level of the skin, including the amount of moisture, dryness, flaking, or oiliness in the skin. In some embodiments, the preselected biomarkers genetically associated with skin hydration include biomarkers that are mapped within one or more genes selected from FLG genes and AQP3 gene.

In some embodiments, the preselected biomarkers genetically associated with skin hydration include biomarkers that are mapped within an FLG gene. In the some embodiments, the biomarkers mapped within an FLG gene can include rs558269137, rs61816761, rs138726443, rs150597413, rs397507563, rs200519781, and/or combinations thereof.

In some embodiments, the preselected biomarkers genetically associated with skin hydration include biomarkers that are mapped within the AQP3 gene. In the some embodiments, the biomarkers mapped within the AQP3 gene can include rs17553719.

### Skin Inflammation and Allergy Risk

The term “skin inflammation” is used herein to include a localized physical condition in which part of the skin becomes reddened, swollen, hot, or painful, including as a reaction to exposure or injury. Common types of skin inflammation include eczema, atopic dermatitis, psoriasis, contracted dermatitis, and Rosacea. In some embodiments, the preselected biomarkers genetically associated with skin inflammation include biomarkers that are mapped within one or more genes selected from the following genes: FLG, HLA-C, IL12B, IL23R, TNIP1, IL13, MTHFR, the intergenic region between HLA-DRA and BTNL2, the intergenic region between PRELID2 and KCTD16, TNFAIP3, and/or combinations thereof.

In some embodiments, the preselected biomarkers genetically associated with skin inflammation and allergy risk include biomarkers that are mapped within an FLG gene. In the some embodiments, the biomarkers mapped within an FLG gene can include rs558269137, rs61816761, rs150597413, rs397507563, and/or combinations thereof.

In some embodiments, the preselected biomarkers genetically associated with skin inflammation and allergy risk include biomarkers that are mapped within the HLA-C gene. In the some embodiments, the biomarkers mapped within the HLA-C gene can include rs12191877.

In some embodiments, the preselected biomarkers genetically associated with skin inflammation and allergy risk include biomarkers that are mapped within the IL12B gene. In the some embodiments, the biomarkers mapped within the IL12B gene can include rs2082412.

In some embodiments, the preselected biomarkers genetically associated with skin inflammation and allergy risk include biomarkers that are mapped within the IL23R gene. In the some embodiments, the biomarkers mapped within the IL23R gene can include rs2201841.

In some embodiments, the preselected biomarkers genetically associated with skin inflammation and allergy risk include biomarkers that are mapped within the TNIP1 gene. In the some embodiments, the biomarkers mapped within the TNIP1 gene can include rs17728338.

In some embodiments, the preselected biomarkers genetically associated with skin inflammation and allergy risk include biomarkers that are mapped within the IL13 gene. In the some embodiments, the biomarkers mapped within the IL13 gene can include rs20541.

In some embodiments, the preselected biomarkers genetically associated with skin inflammation and allergy risk include biomarkers that are mapped within the intergenic region between HLA-DRA and BTNL2. In the some embodiments, the biomarkers mapped within the intergenic between HLA-DRA and BTNL2 can include rs763035.

In some embodiments, the preselected biomarkers genetically associated with skin inflammation and allergy risk include biomarkers that are mapped within the intergenic region between PRELID2 and KCTD16. In the some embodiments, the biomarkers mapped within the intergenic region between PRELID2 and KCTD16 can include rs111314066.

In some embodiments, the preselected biomarkers genetically associated with skin inflammation and allergy risk include biomarkers that are mapped within the TNFAIP3 gene. In the some embodiments, the biomarkers mapped within the TNFAIP3 gene can include rs610604.

In some embodiments, the preselected biomarkers genetically associated with skin inflammation and allergy risk can include one or more of rs138726443, 1249insG (HGMD CI083373), rs374588791 (7264G⁻⁻>T), rs200519781, rs121909626, rs540453626 (8666C⁻⁻>G), rs578153418 (8667C⁻⁻>A), rs761212672 (9887C⁻⁻>A), S2889X (HGMD CX082304), and/or combinations thereof. In some embodiments, the skin inflammation is atopic dermatitis. The chromosome coordinates for the foregoing biomarkers are as follows:
rs374588791 (7264G⁻⁻>T): Chromosome 1, Start: 152280097; End: 152280098.
rs578153418 (8667C⁻⁻>A): Chromosome 1, Start: 152278694; End: 152278695.
rs540453626: Chromosome 1, Start: 152278695; End: 152278696.
1249insG (CI083373): Chromosome 1, Start: 152286113; End: 152286114.
rs761212672 (9887C⁻⁻>A): Chromosome 1, Start: 152277474; End: 152277475.

### Skin Oxidation Protection and Skin Glycation Risk

The term “skin oxidation” is used herein to include any one or more of a number of naturally occurring chemical processes, which involve reaction of the oxygen molecules with other substances which come in contact with it, and which may have an effect on the appearance or consistency of an area of skin, and includes the process by which damage is caused to portion of the skin, including cell membranes and other structures including cellular proteins, lipids and DNA. Human skin is exposed to free-radicals and reactive oxygen species (ROS) caused by solar radiation, air, and environmental pollutants in addition to our own metabolism. ROS in the skin causes oxidative stress, one of the main causes of collagen and elastin degradation that result in wrinkles and sagging of the skin. The only defenses of the skin are its endogenous protection (natural skin pigmentation, ROS-scavenging enzymes) and the antioxidants an individual consumes in his or her diet (e.g., vitamin A, C, E).

The term “skin glycation” is used herein to include any one or more of a number of naturally occurring chemical processes involving glycation, the result of typically covalent bonding of a protein or lipid molecule with a sugar molecule, such as fructose or glucose, without the controlling action of an enzyme, which may have an effect on the skin, including collagen in the skin, including the creation of advanced glycation end products (AGEs).

In some embodiments, the preselected biomarkers genetically associated with skin oxidation protection and skin glycation risk include biomarkers that are mapped within one or more genes selected from the following genes: SOD2, GPX1, CAT, NQO1, GLO1, AGER, and/or combinations thereof.

In some embodiments, the preselected biomarkers genetically associated with skin oxidation and skin glycation include biomarkers that are mapped within the SOD2 gene. In the some embodiments, the biomarkers mapped within the SOD2 gene can include rs4880.

In some embodiments, the preselected biomarkers genetically associated with skin oxidation include biomarkers that are mapped within the GPX1 gene. In the some embodiments, the biomarkers mapped within the GPX1 gene can include rs1050450.

In some embodiments, the preselected biomarkers genetically associated with skin oxidation include biomarkers that are mapped within the promoter region of CAT gene. In the some embodiments, the biomarkers mapped within the promoter region of CAT gene can include rs1001179.

In some embodiments, the preselected biomarkers genetically associated with skin oxidation include biomarkers that are mapped within the promoter region of NQO1 gene. In the some embodiments, the biomarkers mapped within the promoter region of NQO1 gene can include rs1800566, rs2917666, and/or combinations thereof.

In some embodiments, the preselected biomarkers genetically associated with skin glycation include biomarkers that are mapped within the GLO1 gene. In the some embodiments, the biomarkers mapped within the GLO1 gene can include rs1130534, rs1049346, and/or combinations thereof.

In some embodiments, the preselected biomarkers genetically associated with skin glycation include biomarkers that are mapped within the AGER gene. In the some embodiments, the biomarkers mapped within the AGER gene can include rs1800624, rs1800625, rs2070600, and/or combinations thereof.

As a non-limiting example, an assessment table is provided below in TABLE 1.

TABLE 1
Skin Health Characteristics and Exemplary Genes

| Main | Phenotype | | | | | | |
|---|---|---|---|---|---|---|---|
| Characteristic | Sub 1 | Sub 2 | Sub 3 | Sub 4 | Sub 5 | Sub 5 | Genes |
| Skin | Tanning | Sun Spots | Freckles | Wrinkles | | | MC1R, TYR, |
| Photoaging | Response | (Lentigines) | (Ephelides) | and | | | SLC45A2 (MATP), |
| | | | | Collagen | | | SLC24A5, ASIP |
| | | | | Degra- | | | Region, HERC2, |
| | | | | dation | | | IRF4, EXOC2, |
| | | | | | | | STXBP5L, 6p25.3, |
| | | | | | | | MMP1, NCOA6 |
| Skin Texture | Cellulite | Stretch Marks | Varicose | | | | ACE, HIF1A, ELN, |
| and Elasticity | | (Striae | Veins | | | | SRPX, HMCN1, |
| | | Distensae) | | | | | TMEM18, MTHFR |
| Skin Moisture | Dry Skin | Hydration | | | | | FLG, AQP3 |
| Factor | (Ichthyosis) | | | | | | |
| Skin | Eczema | Contact | Psoriasis | Rosacea | | | FLG, HLA-C, IL12B, |
| Inflammation | (Atopic | Dermatitis | | | | | IL23R, TNIP1, IL13, |
| and Allergy | Dermatitis) | | | | | | TNFAIP3, MTHFR, |
| Risk | | | | | | | Intergenic between |
| | | | | | | | HLA-DRA and |
| | | | | | | | BTNL2, Intergenic |
| | | | | | | | between PRELID2 |
| | | | | | | | and KCTD16 |
| Skin Oxidation | Antioxidation | | | | | | SOD2, GPX1, CAT, |
| Protection | Response | | | | | | NQO1 |
| Skin Glycation | Glycation | | | | | | GLO1, AGER |
| | Protection | | | | | | |
| Skin | Vit A | Vit B2 | Vit B6 | Vit B12 | Vit B3 | Vit C | GC, SLC23A1, |
| Nutritional | deficiency | deficiency | deficiency | deficiency | deficiency | deficiency | MTHFR, NBPF3, |
| Needs | Vit D | Vit E | Omega-3/ | GC gene | Best Diet | | FUT2, BCMO1, |
| | deficiency | deficiency | Omega-6 | | | | FADS1, GC genes, |
| | | | deficiency | | | | intergenic region |
| | | | | | | | near APOA5 |

TABLE 2sets forth anon-limiting example of at least a portion of information included in a personalized genetic profile report that can be generated and displayed at one or more displays or computing systems, or alternatively printed in physical form for the individual and/or physician's review, according to at least some embodiments of the methods and systems disclosed herein. In some embodiments, the report may list the relative strength of one or more biomarkers in predicting and/or affecting the likelihood of exhibiting one or more phenotypic attributes. For example, the report may list relative stengths of the one or more biomarkers on a scale from one to four. The scale may range between any numbers, such as, ranges having 0 or 1 as a lower end, which is paired with any of 3, 4, 5, 6, 7, 8, 9, 10, 20, 30, 40, 50, 75, or 100. In some embodiments, a personalized genetic profile report contains genotypic information relevant to the individual's likelihood of exhibiting the one or more (or a plurality of) skin phenotypic attributes (similar to what is shown in TABLE 2), and recommendations in relation to personalized skin care regimens and dietary regimens based on the personalized genetic profile report. For example, a recommendation in relation to the individual's dietary regimen can be assigned to one of the categories below:

1. Optimized Intake,

2. Stay Balance,

3. Minimized Intake.

TABLE 2
Example Information Included in a Personalized Genetic Profile

| | | | | | |
|---|---|---|---|---|---|
| PHENOTYPE | GENETIC | | | | |
| NAME | RESULTS | PAGE # | GENE/LOCUS | MARKER | GENOTYPE |

SKIN PHOTOAGING

| | | | | | |
|---|---|---|---|---|---|
| WRINKLES | INCREASED | P.8 | MMP1 | rs1799750 | TC/T |
| AND | RISK | | | | |
| COLLAGEN | | | | | |
| DEGRADATION | | | | | |
| TANNING | REDUCED | P.9 | EXOC2 | rs12210050 | C/C |
| RESPONSE | | | HERC2 | rs12913832 | A/G |
| | | | intergenic | rs1015362 | C/C |
| | | | intergenic | rs4911414 | G/G |
| | | | IRF4 | rs12203592 | C/C |
| | | | MC1R | rs1805007 | C/C |
| | | | MC1R | rs1805008 | C/C |
| | | | NCOA6 | rs4911442 | A/A |
| SUN SPOTS | NORMAL | P.9 | IRF4 | rs12203592 | C/C |
| (LENTIGINES) | RISK | | MC1R | rs885479 | G/G |
| | | | MC1R | rs1110400 | T/T |
| | | | MC1R | rs1805005 | G/G |
| | | | MC1R | rs1805006 | C/C |
| FRECKLES | NORMAL | P.10 | intergenic | rs1540771 | C/C |
| (EPHELIDES) | RISK | | intergenic | rs4911414 | G/G |
| | | | IRF4 | rs12203592 | C/C |
| | | | MC1R | rs1805007 | C/C |
| | | | MC1R | rs1805008 | C/C |

SKIN TEXTURE AND ELASTICITY

| | | | | | |
|---|---|---|---|---|---|
| CELLULITE | INCREASED | P.10 | ACE | rs4646994/ | D/D |
| | RISK | | | rs1799752 | |
| STRETCH | INCREASED | P.11 | ELN HMCN1 | rs7787362 | T/C C/G |
| MARKS | RISK | | | rs10798036 | |
| (STRIAE | | | | | |
| DISTENSAE) | | | | | |
| VARICOSE | INCREASED | P.11 | MTHFR | rs1801131 | T/G |
| VEINS | RISK | | | | |

SKIN INFLAMMATION AND ALLERGY RISK

| | | | | | |
|---|---|---|---|---|---|
| ROSACEA | INCREASED | P.12 | intergenic | rs763035 | A/A |
| | RISK | | | | |
| CONTACT | NORMAL | P.12 | FLG | rs61816761 | G/G |
| DERMATITIS | RISK | | | | |
| GENERALIZED | HIGH RISK | P.13 | HLA-C | rs1265181 | C/C |
| PSORIASIS | | | HLA-C | rs12191877 | C/C |
| | | | IL12B | rs2082412 | G/G |
| | | | IL13 | rs20541 | G/G |
| ECZEMA | NORMAL | P.13 | FLG | FLG:1249insG | A/A |
| (ATOPIC | RISK | | FLG | FLG:S2889X | TGG/TGG |
| DERMATITIS) | | | FLG | rs61816761 | G/G |
| | | | FLG | rs121909626 | G/G |
| | | | FLG | rs138726443 | G/G |
| | | | FLG | rs150597413 | G/G |

A physician may use the results of the genotyping analysis disclosed herein to, among other things: 1) help determine the treatment regimen based on skin condition, lifestyle, etc.; 2) analyze skin care routine and what is working; 3) recommend actions steps to enhance skin health; 4) leverage products to sell to the patient; and 5) provide nutrigenomics guidance based on the specific diet type that optimizes skin health.

For example, information obtained using the diagnostic assays described herein is useful for determining if an individual will respond to treatment for a given indication. Based on the prognostic information, a doctor can recommend a therapeutic protocol, useful for prescribing different treatment protocols for a given individual.

In addition, knowledge of the identity of a particular allele in an individual (the gene profile) allows customization of therapy for a particular condition to the individual's genetic profile. For example, an individual's genetic profile can enable a doctor: 1) to more effectively prescribe a drug that will address the molecular basis of the disease or condition; 2) to better determine the appropriate dosage of a particular drug and 3) to identify novel targets for drug development. Expression patterns of individual patients can then be compared to the expression profile of the disease to determine the appropriate drug and dose to administer to the patient.

The ability to target populations expected to show the highest clinical benefit, based on genetic profile, can enable: 1) the repositioning of marketed drugs/therapeutic options with disappointing market results; 2) the rescue of drug candidates/therapeutic options whose clinical development has been discontinued as a result of safety or efficacy limitations, which are patient subgroup specific; and 3) an accelerated and less costly development for drug/therapeutic candidates and more optimal drug/therapeutic labeling.

Side effects of a particular treatment are those related to treatment based on a positive correlation between frequency or intensity of occurrence and treatment, whether by drug or therapeutic. Such information is usually collected in the course of studies on efficacy of a treatment and many methods are available to obtain such data. Resulting information is widely distributed among the medical profession and patients receiving treatment.

A treatment result is defined here from the point of view of the treating doctor, who judges the efficacy of a treatment as a group result. Within the group, individual patients can recover completely and some may even worsen, due to statistical variations in the course of the disease or phenotypic attribute and the patient population. Some patients may discontinue treatment due to side effects, in which case no improvement in their condition can occur. An improved treatment result is an overall improvement assessed over the whole group. Improvement can be solely due to an overall reduction in frequency or intensity of side effects. It is also possible that doses can be increased or the dosing regimen can be stepped up faster thanks to less troublesome side effects in the group and consequently an earlier onset of recovery or better remission of the disease.

A disorder or phenotypic attribute, which is responsive to treatment with a particular drug, therapeutic, or treatment, is defined to be a disorder or phenotypic attribute, which is, according to recommendations in professional literature and drug formularies, known to respond with at least partial remission of the symptoms to a treatment with such drug, therapeutic, or treatment. In most countries such recommendations are subject to governmental regulations, allowing and restricting the mention of medical indications in package inserts. Other sources are drug formularies of health management organizations. Before approval by governmental agencies certain recommendations can also be recognized by publications of confirmed treatment results in peer reviewed medical journals. Such collective body of information defines what is understood here to be a disorder that is responsive to treatment with a particular medication. Being responsive to particular treatment does not exclude that the disorder or phenotypic attribute in an individual patient can resist treatment with such treatment, as long as a substantial portion of persons having the disorder or phenotypic attribute respond with improvement to the treatment.

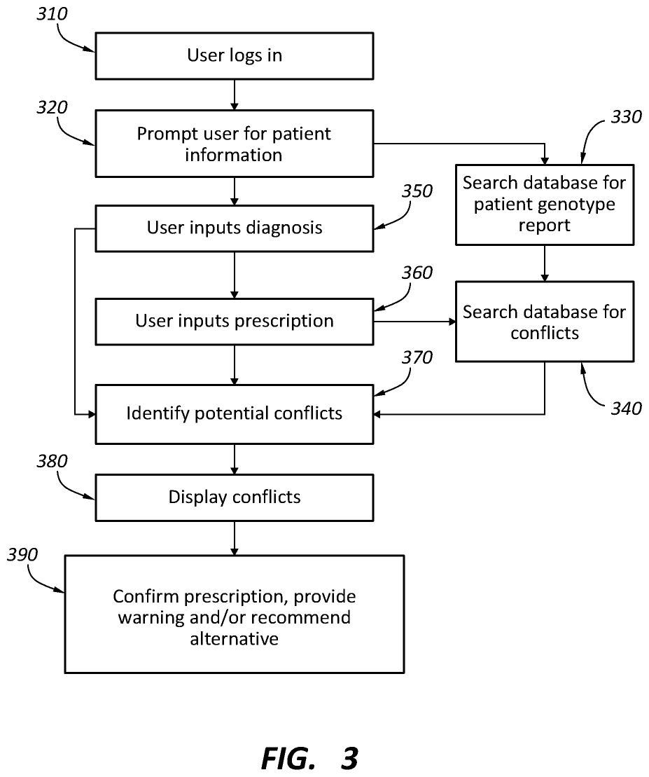

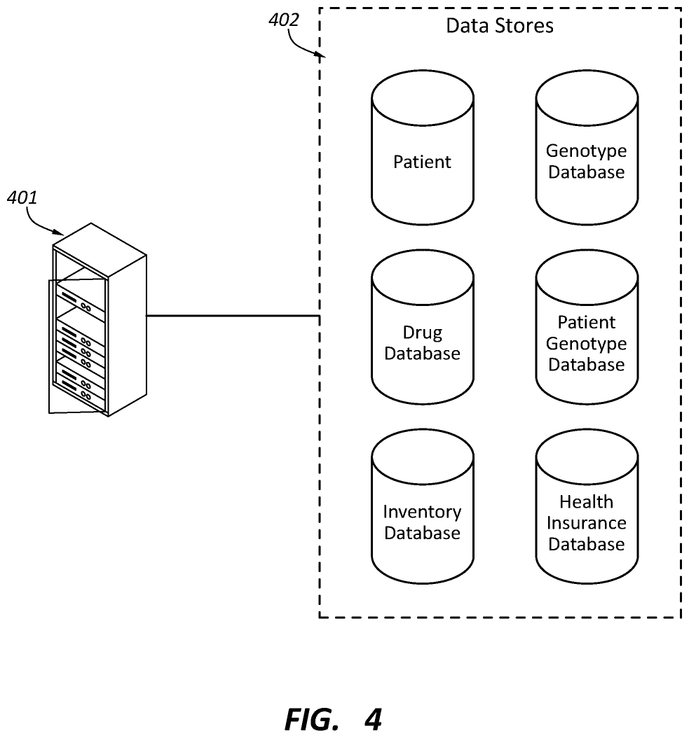

Some embodiments provide a method and system for e.g. a designated user to access information about the genetic profile of an individual to recommend or warn about particular treatments. In some embodiments, the user is typically a healthcare provider. FIG. 3 displays an interactive process of a healthcare provider, or individual with the application system for recommending particular skin therapeutic regimen. A healthcare provider can access information 310 of their patient by accessing the system and interacting with the patient genetic records. As the system is targeted to providing personal information, the system will require the identity of the individual 320 to analyze or report upon. This information may be accessed 330 through information stored onsite or offsite in, for example, a patient data warehouse or with a laboratory or company providing such services. Either the system and/or the healthcare provider can provide additional information such as the diagnosis 350 (e.g., the genotyping may consist of analyzing an individual to detect genetic anomalies associated with the disorder, disease, or phenotypic attribute). Further, the healthcare provider can input any recommended prescriptions 360 that can be analyzed 340 against the individual's genetic profile to determine the efficacy and/or risk of such a treatment protocol. Any potential conflicts and problems can be flagged 370 and displayed 380 for the healthcare provider to review. Alternatively, the system can recommend or warn against particular medications and treatments, or classes of medications or treatments upon analysis of the individual's genetic profile report. Once any warnings or recommendations are made, the system can further confirm the determination of the healthcare provider and provide additional warnings or alternative medications or treatments 390. The system 401 can be tied, as shown in FIG. 4, into one or more additional databases 402 to further analyze inventory, price, insurance restrictions, genotype, and the like.

### E. Nutritional Attributes Associated with Skin Health

An important component in preserving or restoring the skin health of an individual is the identification and/or correction of nutrient deficiencies. In many instances, skin-related diseases and conditions may be treated or prevented due to their linkage to nutrient imbalances. For people seeking to improve their outward appearance, nutrient deficiencies are reflected in the skin, eyes, hair and other outward indicators in a person's body.

In some embodiments disclosed herein, tissue, blood, and serum tests measuring quantities of individual nutrients are used to determine an individual's nutrient deficiencies. Diet and nutrient uptake is one of the many factors that influence the nutrient status of an individual. Insufficient intake or uptake of specific nutrients generally results in a deficiency of that nutrient. According to some embodiments, the level of one or more of vitamins A, B1, B2, B3, B6, B5, B12, D, and E, folic acid, folate, Biotin, omega-3 fatty, and omega-6 fatty acid can be tested in nutrient testing assays.

In addition, or alternatively, there are many other factors beyond diet that determine adequate nutrition conditions of an individual. This is because individuals are biochemically unique. Nutrient deficiencies vary between individuals and do not necessarily correlate directly with nutrient intake, even among those with similar health conditions. These factors include genetic predisposition, biochemical individuality, nutrient absorption and metabolism, age, disease conditions, and medications. Assays providing information regarding an individual's nutrient status in correlation with such factors may therefore also be included as a nutrient deficiency assay.

In some embodiments, the methods and systems disclosed herein includes at least one type of nutrient deficiency assay that provides information regarding nutrient deficiencies within an individual. These functional deficiency assays report defects in the biochemical pathways that depend upon the optimal function of the nutrients. A deficient or defective metabolic pathway may operate at a sub-optimal level for many months or even years before a clinical symptom may become apparent, if they become apparent at all. The term “functional deficiency”, as used herein, includes anything that may reduce the concentration or the efficacy of a nutrient as compared with a normal range within a population. With a deficiency, a nutrient may be present, but it may not be properly activated, localized, or have sufficient cofactors to function at a normal level of activity. Functional deficiencies include inefficiencies or deficiencies in intracellular activation, storage concentration or activity of cofactors, and tissues with increased metabolic needs. Non-limiting examples include inefficient absorption by the gastrointestinal tract, deficient transport to the appropriate tissue, impeded transport through the cell membrane, presence of intracellular inhibitors, and flaws in the biochemical pathways for the uptake of nutrients.

In some embodiments, the nutrient deficiency assay is an assay measuring levels of accumulation of the nutrient in suitable cell types, such as lymphocytes, of the individual. By way of exemplification, an intracellular function assay is generally used and comprises the steps of collecting lymphocyte cells, isolating the cells from other whole blood components, and maintaining the cells in culture during the assay. The lymphocytes collected have a 4- to 6-month lifespan in which nutrients are accumulated. The resting lymphocytes are stimulated to undergo cell division and grow in culture. The degree to which the lymphocytes grow having various nutrients available is directly related to the nutrient levels accumulated in the lymphocytes. For example, if the lymphocytes are able to grow in an environment deficient in vitamin C, then the lymphocyte has efficiently uptakes and stored vitamin C prior to harvest. On the other hand, if the lymphocyte is unable to grow in the absence of vitamin C, a deficiency is indicated. From the lymphocyte's degree of growth, a functional intracellular analysis of a broad range of nutrient deficiencies may be obtained.

The nutrient targeted in the nutrient deficiency assay can generally be any nutrient and can, for example, be selected from vitamins, minerals, amino acids, antioxidants, and metabolites. In some embodiments, the nutrient is a vitamin such as vitamin A, B1, B2, B3, B6, B12, D, E, biotin, folate, and pantothenate; minerals such as calcium, magnesium, selenium, and zinc; an amino acid such as asparagine, carnitine, glutamine, and serine; an antioxidant selected from coenzyme Q10, glutathione, and cysteine; or a metabolite such as lipoic acid, oleic acid, choline, inositol, fructose, glucose, and insulin. In some embodiments, the target nutrient is selected from folate, folic acid, Vitamin A, Vitamin B2, Vitamin B6, Vitamin B12, Vitamin B3, Vitamin C, Vitamin D, Vitamin E, omega-3 fatty acid, omega-6 fatty acid, and/or combinations thereof.

In some embodiments, genetic testing is used to acquire information regarding an individual's skin health based on the individual's genetic conditions. One of skill in the art will readily appreciate that an individual's inherited skin health risks and potential skin health problems can be assessed through the genetic testing. More importantly, correlations may be drawn to nutrient deficiencies based on sets of previously observed genetic variations and nutrient deficiencies. Thus, genetic testing assays may lead to information regarding the cause of nutrient deficiencies or nutrient deficiencies that are unobserved in other assay methods. This allows for the development of a suitable diet, lifestyle, and supplement regimen that matches the unique nutrient deficiencies of each individual. The detection of genetic variations assay can be achieved by using any one of the methodologies and systems described above. In one exemplification, assays can be carried out to detect genetic variations of a nucleotide sequence of a gene, or of the amino acid sequence of a protein encoded by such gene, which may affect the way an individual's body responds to certain stimuli such as damage, infection, or even nutrient intake. Based on the genetic test results, a personalized skin care regimen may be developed and/or implemented for the individual.

In some embodiments, functional assessment tests may also be conducted to monitor if an individual's deficiencies are related to breakdown in the uptake pathways or if cofactors or related biomolecule deficiencies are actually the root cause of an observed deficiency. For example, a calcium deficiency maybe observed in an individual. However, the root cause of the deficiency may breakdown in the conversion of vitamin D to 1,25-dihydroxyvitamin D, which is necessary in the production of TRPV6—a protein necessary for calcium absorption in the intestine. Functional assessment tests may be used to determine if biochemical pathways are functioning inefficiently and target growth factors and other active ingredients that induce correct function of the pathways. Thus, deficiencies may be identified not only from the perspective of intake of nutrients, but also efficiency of uptake of available nutrients.

Biomarkers Associated with Skin Nutrition

In some embodiments, the preselected biomarkers genetically associated with skin nutrition include biomarkers that are mapped within one or more genes selected from SLC23A1, MTHFR, NBPF3, FUT2, BCMO1, FADS1, GC genes, the intergenic region near APOA5, and/or combinations thereof.

In some embodiments, the preselected biomarkers genetically associated with skin nutrition include biomarkers that are mapped within a GC gene. In the some embodiments, the biomarkers mapped within a GC gene can include rs2282679. In some embodiments, genetic variations identified in the biomarker rs2282679 are associated with deficiency in levels of Vitamin D.

In some embodiments, the preselected biomarkers genetically associated with skin nutrition include biomarkers that are mapped within the SLC23A1 gene. In the some embodiments, the biomarkers mapped within the SLC23A1 gene can include rs33972313. In some embodiments, genetic variations identified in the biomarker rs33972313 are associated with deficiency in levels of Vitamin C.

In some embodiments, the preselected biomarkers genetically associated with skin nutrition include biomarkers that are mapped within the MTHFR gene. In the some embodiments, the biomarkers mapped within the MTHFR gene can include rs1801133 and/or rs1801131. In some embodiments, genetic variations identified in the biomarker rs1801133 and/or rs1801131 are associated with deficiency in levels of Vitamin B2, folate, folic acid, and/or combinations thereof.

In some embodiments, the preselected biomarkers genetically associated with skin nutrition include biomarkers that are mapped within the NBPF3 gene. In the some embodiments, the biomarkers mapped within the NBPF3 gene can include rs4654748. In some embodiments, genetic variations identified in the biomarker rs4654748 are associated with deficiency in levels of Vitamin B6.

In some embodiments, the preselected biomarkers genetically associated with skin nutrition include biomarkers that are mapped within the FUT2 gene. In the some embodiments, the biomarkers mapped within the FUT2 gene can include rs602662. In some embodiments, genetic variations identified in the biomarker rs602662 are associated with deficiency in levels of Vitamin B12.

In some embodiments, the preselected biomarkers genetically associated with skin nutrition include biomarkers that are mapped within the BCMO1 gene. In the some embodiments, the biomarkers mapped within the BCMO1 gene can include rs7501331, rs12934922, and/or combinations thereof. In some embodiments, genetic variations identified in the biomarker rs7501331, rs12934922, and/or combinations thereof are associated with deficiency in Vitamin A.

In some embodiments, the preselected biomarkers genetically associated with skin nutrition include biomarkers that are mapped within the FADS1 gene. In the some embodiments, the biomarkers mapped within the FADS1 gene can include rs174547. In some embodiments, genetic variations identified in the biomarker rs174547 is associated with deficiency in levels of omega-3 fatty acids, omega-6 fatty acids, and/or combinations thereof.

In some the preselected biomarkers genetically associated with skin nutrition include biomarkers that are mapped within the intergenic region near APOA5. In the some embodiments, the biomarkers mapped within the intergenic region near APOA5 can include rs12272004. In some embodiments, genetic variations identified in the biomarker rs12272004 is associated with deficiency in Vitamin E.

In some embodiments of disclosed herein, the determination of the likelihood of the individual to exhibit the one or more (or a plurality of) skin phenotypic attributes is further based on one or more additional criteria. These additional criteria can include, among others, base pair sequence homology to another known genetic marker sequence of interest; the presence of two or more regions of DNA on the same chromosome or genetic marker (i.e., synteny); relevance to the description of the molecular function, biological process and cellular component of the protein coded by the gene under investigation (i.e., ontology) and ontological classification; conservation of mutated sequence sites at conserved or less conserved sequence homology sites in the genome; quality of research on the genotype, genetic marker and phenotype under investigation; biological significance of the genetic variation and/or biomarker (for example, whether the genetic variation specifies a protein coding change); and regulatory value and classifications of the amino acid(s) specified by the genetic variation. In some particular embodiments, the one or more additional criteria is selected from the group consisting of nucleotide sequence homology, expression level, enzyme activity, relative synteny among the preselected biomarkers, family history, ontological relevance, quality of supporting research, degree of phenotypic significance, and/or combinations thereof.

### F. Methods

In some embodiments disclosed herein, the genetic variations, polymorphism patterns, or genetic profiles can be identified by detecting one or more component genetic variations in a biological sample derived from an individual, by using any one of a variety of systems and techniques available. In some embodiments, detection of a genetic variation includes, but not limited to, amplification of a sequence with specific primers; determination of the nucleotide sequence of the nucleic acid sample; hybridization analysis; single strand conformational polymorphism analysis; denaturing gradient gel electrophoresis; mismatch cleavage detection; and the like. Detection of a genetic variation can also be accomplished by detecting an alteration in the level of a mRNA transcript of the gene; aberrant modification of the corresponding gene, e.g., an aberrant methylation pattern; the presence of a non-wild-type splicing pattern of the corresponding mRNA; an alteration in the level of the corresponding polypeptide; determining the electrophoretic mobility of the allele or fragments thereof (e.g., fragments generated by endonuclease digestion), and/or an alteration in corresponding polypeptide activity.

In some embodiments, an individual can be genotyped for a genetic variation, more preferably a polymorphism, by collecting and assaying a biological sample of the individual, the biological sample having nucleic acid, to determine the nucleotide sequence of the gene at that polymorphism, the amino acid sequence encoded by the gene at that polymorphism, or the concentration of the expressed product, e.g., by using one or more genotyping reagents, such as but not limited to nucleic acid reagents, including primers, etc., which may or may not be labeled, amplification enzymes, buffers, etc. In certain embodiments, the target polymorphism will be detected at the protein level, e.g., by assaying for a polymorphic protein. In yet other embodiments, the target polymorphism will be detected at the nucleic acid level, e.g., by assaying for the presence of nucleic acid polymorphism such as, e.g., a single nucleotide polymorphism (SNP) that cause expression of the polymorphic protein.

In some embodiments of the methods disclosed herein, the acquiring knowledge of one or more genetic variation (or other complementary action such as determining one or more characteristics of a genetic variation) comprises an analytical assay which can generally be any analytical assay known to those of skill in the art and can be, for example, an antibody-based assay, a nucleotide-based assay, or an enzymatic activity assay. Non-limited examples of suitable analytical nucleotide-based assays include nucleic acid sequencing, polypeptide sequencing, restriction digestion, capillary electrophoresis, nucleic acid amplification-based assays, nucleic acid hybridization assay, comparative genomic hybridization, real-time PCR, quantitative reverse transcription PCR (qRT-PCR), PCR-RFLP assay, HPLC, mass-spectrometric genotyping, fluorescent in-situ hybridization (FISH), next generation sequencing (NGS), and any combination thereof. Other non-limiting examples of suitable analytical antibody-based assays include ELISA, immunohistochemistry, western blotting, mass spectrometry, flow cytometry, protein-microarray, immunofluorescence, a multiplex detection assay, and any combination thereof.

In some embodiments of the methods disclosed herein, depending on the genetic variations being studied, the acquiring knowledge of one or more genetic variation comprises a nucleic acid-based analytical assay performed on a nucleic acid sample obtained from an individual, where the analytical assay can include one or more of the following techniques: nucleic acid sequencing, polypeptide sequencing, restriction digestion, capillary electrophoresis, nucleic acid amplification-based assays, nucleic acid hybridization assay, comparative genomic hybridization, real-time PCR, quantitative reverse transcription PCR (QRT-PCR), PCR-RFLP assay, HPLC, mass-spectrometric genotyping, fluorescent in-situ hybridization (FISH), next generation sequencing (NGS), or a combination Thereof.

Genetic variations, polymorphism patterns, or genetic profiles can be identified by detecting one or more component genetic variations using a technique that includes 1) performing a hybridization reaction between a nucleic acid sample and a probe that is capable of hybridizing to the genetic variation; 2) sequencing at least a portion of the genetic variation; or 3) determining the electrophoretic mobility of the genetic variation or fragments thereof (e.g., fragments generated by endonuclease digestion). In some embodiments, the genetic variations determined as described above can optionally be subjected to an amplification step prior to performing the identification step.

Accordingly, in some embodiments disclosed herein, the analytical assay is an electrophoretic mobility assay in which a nucleic acid sequence comprising at least one of the genetic variations is detected by amplifying the nucleic acid region corresponding to said at least one genetic variations and comparing the electrophoretic mobility of the amplified nucleic acid to the electrophoretic mobility of the corresponding region in a reference individual that does not comprise said at least one genetic variations.

In some embodiments, the nucleic acid-based analytical assay can be an allele-specific polymerase chain reaction or a next-generation sequencing method. In some embodiments, preferred amplification methods can be selected from the following methodologies: polymerase chain reaction (PCR), ligase chain reaction (LCR), strand displacement amplification (SDA), cloning, and variations of the above (e.g., RT-PCR, quantitative reverse transcription PCR (qRT-PCR), allele specific amplification, PCR-RFLP assay). Oligonucleotides necessary for amplification may be selected, for example, from within the metabolic gene locus, either flanking the marker of interest (as required for PCR amplification) or directly overlapping the biomarker (as in allele specific oligonucleotide (ASO) hybridization). In some preferred embodiments, the sample is hybridized with a set of primers, which hybridize 5′ and 3′ in a sense or antisense sequence to the skin health associated alleles, and is subjected to a PCR amplification. Genomic DNA or mRNA can be used directly or indirectly, for example, to convert into cDNA. In addition or alternatively, the region of interest can be cloned into a suitable vector and grown in sufficient quantity for analysis.

In some embodiments, the analytical assay used to acquire the knowledge of the one or more genetic alterations associated with each member of a first set and a second set of preselected biomarkers in the individual involves a next generation sequencing procedure. As used herein “next-generation sequencing” refers to oligonucleotide sequencing technologies that have the capacity to sequence oligonucleotides at speeds above those possible with conventional sequencing methods (e.g. Sanger sequencing), due to performing and reading out thousands to millions of sequencing reactions in parallel. Non-limiting examples of next-generation sequencing methods/platforms include Massively Parallel Signature Sequencing (Lynx Therapeutics); solid-phase, reversible dye-terminator sequencing (Solexa/Illumina); DNA nanoball sequencing (Complete Genomics); SOLiD technology (Applied Biosystems); 454 pyro-sequencing (454 Life Sciences/Roche Diagnostics); ion semiconductor sequencing (ION Torrent); and technologies available from Pacific Biosciences, Intelligen Bio-systems, Oxford Nanopore Technologies, and Helicos Biosciences.

Accordingly, in some embodiments, the NGS procedure used in the methods disclosed herein can comprise pyrosequencing, sequencing by synthesis, sequencing by ligation, or a combination of any thereof. In some embodiments, the NGS procedure is performed by an NGS platform selected from Illumina, Ion Torrent, Qiagen, Invitrogen, Applied Biosystem, Helicos, Oxford Nanopore, Pacific Biosciences, and Complete Genomics. In some embodiments, the next generation sequencing procedure is performed on a MiSeq platform or NextSeq platform (Illumina).

In some embodiments, the analytical assay is a gene expression assay performed to determine whether the expression of one or more biomarkers is altered in the individual.

In some embodiments disclosed herein, the analytical assay is a nucleic acid hybridization assay that includes contacting nucleic acids from the individual with a nucleic acid probe comprising a nucleic acid sequence complementary to a nucleic acid sequence encoding at least one of said genetic variations and further comprising a detectable label.

A genetic variation may also be detected indirectly, e.g. by analyzing the protein product encoded by the DNA sequence. For example, where the biomarker in question results in the translation of a mutant protein, the protein can be detected by any one of a variety of antibody-based protein detection assays. Such methods include immunodetection and biochemical tests, such as ELISA, immunohistochemistry, western blotting, protein-microarray, immunofluorescence, multiplex detection assay. Also suitable for the methods and systems of the present application is size fractionation, where the protein has a change in apparent molecular weight either through truncation, elongation, altered folding or altered post-translational modifications.

In some embodiments, the methods include collecting biological samples from one or more individuals and exposing the samples to detection assays under conditions such that the presence or absence of at least one genetic variation is revealed. In some embodiments, samples derived from (e.g., obtained from) an individual may be employed. Any biological sample that comprises nucleic acids and/or proteins of interest from the individual is suitable for use in the methods of the application. The biological sample may be processed so as to isolate the nucleic acids and/or proteins of interest. Alternatively, whole cells or other biological samples may be used without isolation of the polynucleotides and/or proteins contained therein.

Nucleic acids can be extracted from the biological sample using conventional techniques. The nucleic acids to be extracted from the biological sample may be DNA, or RNA (e.g., total RNA). Typically RNA is extracted if the genetic variation to be studied is situated in the coding sequence of a gene. Where RNA is extracted from the biological sample, the methods further comprise a step of obtaining cDNA from the RNA. This may be carried out using conventional methods, such as reverse transcription using suitable primers. Subsequent procedures are then carried out on the extracted DNA or the cDNA obtained from extracted RNA. The term DNA, as used herein, may include both DNA and cDNA.

In some embodiments, the genetic variations to be tested are known and characterized, e.g. in terms of sequence. Therefore nucleic acid regions comprising the genetic variations may be obtained using methods known in the art.

In one aspect, DNA regions which contain the genetic variations to be identified (target DNA regions) are subjected to an amplification reaction in order to obtain amplification products that contain the genetic variations to be identified. Any suitable technique or method may be used for amplification. In general, the technique allows the (simultaneous) amplification of all the DNA sequences containing the genetic variations to be identified. In other words, where multiple genetic variations are to be analyzed, it is preferable to simultaneously amplify all of the corresponding target DNA regions (comprising the target genetic variations). In some embodiments, carrying out the amplification in a single step (or as few steps as possible) simplifies the method.

Analyzing a polynucleotide sample can be conducted in a number of ways. Preferably, the allele can optionally be subjected to an amplification step prior to performance of the detection step. Preferred amplification methods are selected from the group consisting of: the polymerase chain reaction (PCR), the ligase chain reaction (LCR), strand displacement amplification (SDA), cloning, and variations of the above (e.g. RT-PCR and allele specific amplification). A test nucleic acid sample can be amplified with primers that amplify a region known to comprise the target polymorphism(s), for example, from within the metabolic gene loci, either flanking the marker of interest (as required for PCR amplification) or directly overlapping the marker (as in allele specific oligonucleotide (ASO) hybridization). In a particularly preferred embodiment, the sample is hybridized with a set of primers, which hybridize 5′ and 3′ in a sense or antisense sequence to the vascular disease associated allele, and is subjected to a PCR amplification. Genomic DNA or mRNA can be used directly or indirectly, for example, to convert into cDNA. Alternatively, the region of interest can be cloned into a suitable vector and grown in sufficient quantity for analysis.

The nucleic acid may be amplified by conventional techniques, such as a polymerase chain reaction (PCR), to provide sufficient amounts for analysis. The use of the polymerase chain reaction is known and described in a variety of publications. Other methods for amplification of nucleic acids is ligase chain reaction (“LCR”), isothermal amplification method, or Strand Displacement Amplification or Repair Chain Reaction (RCR), transcription-based amplification systems (TAS), including nucleic acid sequence based amplification (NASBA) and 3SR, cyclic and non-cyclic synthesis of single-stranded RNA (“ssRNA”), ssDNA, and double-stranded DNA (dsDNA), and di-nucleotide amplification. In addition or alternatively, other suitable amplification methods include: self-sustained sequence replication, transcriptional amplification system, Q-Beta Replicase, or any other nucleic acid amplification method, followed by the detection of the amplified molecules using techniques known to those of skill in the art. These detection schemes are useful for the detection of nucleic acid molecules if such molecules are present in very low numbers.

Once the region of interest has been amplified, the genetic variation of interest can be detected in the PCR product by nucleotide sequencing, by SSCP analysis, or any other method known in the art. In some embodiments, any of a variety of sequencing reactions known in the art can be used to directly sequence at least a portion of the gene of interest and detect allelic variants, e.g., mutations, by comparing the sequence of the sample sequence with the corresponding wild-type (control) sequence. Exemplary sequencing reactions include those based on techniques developed by Maxam and Gilbert. It is also contemplated that any of a variety of automated sequencing procedures can be utilized when performing the subject assays, including by mass spectrometry. It will be evident to one skilled in the art that, for certain embodiments, the occurrence of only one, two or three of the nucleic acid bases need be determined in the sequencing reaction. For instance, A-track or the like, e.g., where only one nucleotide is detected, can be carried out.

The high demand for low-cost sequencing has driven the development of high-throughput sequencing (or next-generation sequencing) technologies that parallelize the sequencing process, producing thousands or millions of sequences concurrently. High-throughput sequencing including ultra-high-throughput sequencing technologies are intended to lower the cost of DNA sequencing beyond what is possible with standard dye-terminator methods. These methods include pyrosequencing, reversible dye-terminator, SOLiD sequencing using sequencing by ligation, ion semiconductor sequencing, Heliscope single molecule sequencing (Helicos Biosciences, single molecule real-time (SMRT) sequencing (Pacific Biosciences), nanopore DNA sequencing, hybridization sequencing, mass spectrometry sequencing, Sanger microfluidic sequencing, microscopy-based techniques such as transmission electron microscopy DNA sequencing, RNA polymerase (RNAP), in intro virus high-throughput sequencing, and the like.

In some embodiments of the present application, sequences of the genetic variations of interest are detected using a PCR-based assay. In some embodiments, the PCR assay comprises the use of oligonucleotide primers that hybridize only to the variant or wild type allele (e.g., to the region of polymorphism or mutation). Both sets of primers are used to amplify a sample of DNA. If only the mutant primers result in a PCR product, then the patient has the mutant allele. If only the wild-type primers result in a PCR product, then the patient has the wild type allele.

In preferred embodiments of the present application, sequences of the genetic variations of interest are detected using a hybridization assay. In a hybridization assay, the presence of absence of a given SNP or mutation is determined based on the ability of the DNA from the sample to hybridize to a complementary DNA molecule (e.g., a oligonucleotide probe). Parameters such as hybridization conditions, polymorphic primer length, and position of the polymorphism within the polymorphic primer may be chosen such that hybridization will not occur unless a polymorphism present in the primer(s) is also present in the sample nucleic acid. Those of ordinary skill in the art are well aware of how to select and vary such parameters.

In some cases, the presence of the specific allele in DNA from an individual can be shown by restriction enzyme analysis. For example, the specific nucleotide polymorphism can result in a nucleotide sequence comprising a restriction site that is absent from the nucleotide sequence of another allelic variant.

In a further embodiment, protection from cleavage agents (such as a nuclease, hydroxylamine or osmium tetroxide and with piperidine) can be used to detect mismatched bases in RNA/RNA DNA/DNA, or RNA/DNA heteroduplexes. In general, the technique of “mismatch cleavage” starts by providing heteroduplexes formed by hybridizing a control nucleic acid, which is optionally labeled, e.g., RNA or DNA, comprising a nucleotide sequence of the allelic variant of the gene of interest with a sample nucleic acid, e.g., RNA or DNA, obtained from a tissue sample. The double-stranded duplexes are treated with an agent which cleaves single-stranded regions of the duplex such as duplexes formed based on base-pair mismatches between the control and sample strands. For instance, RNA/DNA duplexes can be treated with RNase and DNA/DNA hybrids treated with 51 nucleases to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine whether the control and sample nucleic acids have an identical nucleotide sequence or in which nucleotides they are different. In some embodiments, the control or sample nucleic acid is labeled for detection.

Over or under expression of a gene, in some cases, is correlated with a genomic polymorphism. The polymorphism can be present in an open reading frame (coded) region of the gene, in a “silent” region of the gene, in the promoter region, or in the 3′ untranslated region of the transcript. Methods for determining polymorphisms are well known in the art and include, but are not limited to, the methods discussed below.

Detection of point mutations or additional base pair repeats (as required for the polymorphism) can be accomplished by molecular cloning of the specified allele and subsequent sequencing of that allele using techniques known in the art. In addition or alternatively, the gene sequences can be amplified directly from a genomic DNA preparation from the sample using PCR, and the sequence composition is determined from the amplified product. As described more fully below, numerous methods are available for analyzing an individual's DNA for mutations at a given genetic locus such as the gene of interest.

In some embodiments, a detection method is allele specific hybridization using probes overlapping the polymorphic site and having about 5, or alternatively 10, or alternatively 20, or alternatively 25, or alternatively 30 nucleotides around the polymorphic region. In some embodiments of the application, several probes capable of hybridizing specifically to the allelic variant are attached to a solid phase support, e.g., a “chip”. Oligonucleotides can be bound to a solid support by a variety of processes, including lithography. For example a chip can hold up to 250,000 oligonucleotides (GeneChip, Affymetrix). Mutation detection analysis using these chips comprising oligonucleotides, also termed “DNA probe arrays” has been well documented.

In addition or alternatively, also known in the art are various methods that utilize oligonucleotide ligation as a means of detecting polymorphisms.

In some embodiments, alterations in electrophoretic mobility are used to identify the particular allelic variant. For example, single strand conformation polymorphism (SSCP) may be used to detect differences in electrophoretic mobility between mutant and wild type nucleic acids. Single-stranded DNA fragments of sample and control nucleic acids are denatured and allowed to renature. The secondary structure of single-stranded nucleic acids varies according to their nucleotide sequences; therefore the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In some embodiments, the systems and methods disclosed herein can utilize heteroduplex analysis to separate double stranded heteroduplex molecules on the basis of changes in electrophoretic mobility.

In performing SSCP analysis, the PCR product may be digested with a restriction endonuclease that recognizes a sequence within the PCR product generated by using as a template a reference sequence, but does not recognize a corresponding PCR product generated by using as a template a variant sequence by virtue of the fact that the variant sequence no longer contains a recognition site for the restriction endonuclease.

In some embodiments, the identity of the allelic variant is obtained by analyzing the movement of a nucleic acid comprising the polymorphic region in polyacrylamide gels containing a gradient of denaturant, which is assayed using denaturing gradient gel electrophoresis (DGGE). When DGGE is used as the method of analysis, DNA will be modified to insure that it does not completely denature, for example by adding a GC clamp of approximately 40 by of high-melting GC-rich DNA by PCR. In some embodiments, a temperature gradient is used in place of a denaturing agent gradient to identify differences in the mobility of control and sample DNA.

Non-limiting examples of techniques for detecting differences of at least one nucleotide between 2 nucleic acids include selective oligonucleotide hybridization, selective amplification, or selective primer extension. For example, oligonucleotide probes may be prepared in which the known polymorphic nucleotide is placed centrally (allele-specific probes) and then hybridized to target DNA under conditions which permit hybridization only if a perfect match is found. Such allele specific oligonucleotide hybridization techniques may be used for the detection of the nucleotide changes in the polymorphic region of the gene of interest. For example, oligonucleotides having the nucleotide sequence of the specific allelic variant are attached to a hybridizing membrane and this membrane is then hybridized with labeled sample nucleic acid. Analysis of the hybridization signal will then reveal the identity of the nucleotides of the sample nucleic acid.

In addition or alternatively, allele specific amplification technology which depends on selective PCR amplification may be used in conjunction with the instant application. Oligonucleotides used as primers for specific amplification may carry the allelic variant of interest in the center of the molecule (so that amplification depends on differential hybridization) or at the extreme 3′ end of one primer where, under appropriate conditions, mismatch can prevent, or reduce polymerase extension. This technique is also termed “PROBE” for Probe Oligo Base Extension. In addition it may be desirable to introduce a novel restriction site in the region of the mutation to create cleavage-based detection.

In another embodiment, identification of the allelic variant is carried out using an oligonucleotide ligation assay (OLA). The OLA protocol uses two oligonucleotides which are designed to be capable of hybridizing to abutting sequences of a single strand of a target. One of the oligonucleotides is linked to a separation marker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson et al. (1990) Proc. Natl. Acad. Sci. (U.S.A.) 87:8923-8927). In this method, PCR is used to achieve the exponential amplification of target DNA, which is then detected using OLA.

Several techniques based on this OLA method have been developed and can be used to detect the specific allelic variant of the polymorphic region of the gene of interest. For example, an OLA using an oligonucleotide having 3′-amino group and a 5′-phosphorylated oligonucleotide can be deployed to form a conjugate having a phosphoramidate linkage. In another variation, OLA combined with PCR permits typing of two alleles in a single microtiter well. By marking each of the allele-specific primers with a unique hapten, i.e. digoxigenin and fluorescein, each OLA reaction can be detected by using hapten specific antibodies that are labeled with different enzyme reporters, alkaline phosphatase or horseradish peroxidase. This system permits the detection of the two alleles using a high throughput format that leads to the production of two different colors.

In some embodiments, the single base polymorphism can be detected by using a specialized exonuclease-resistant nucleotide, as disclosed, e.g., in Mundy (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In some embodiments of the application, a solution-based method is used for determining the identity of the nucleotide of the polymorphic site. As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA™ is described by Goelet et al. (PCT Appln. No. 92/15712). This method uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087) the method of Goelet et al. supra, is preferably a heterogeneous phase assay, in which the primer or the target molecule is immobilized to a solid phase.

Several primer-guided nucleotide incorporation procedures for assaying polymorphic sites in DNA have been described (Komher et al. (1989) Nucl. Acids. Res. 17:7779-7784; Sokolov (1990) Nucl. Acids Res. 18:3671; Syvanen et al. (1990) Genomics 8:684-692; Kuppuswamy et al. (1991) Proc. Natl. Acad. Sci. (U.S.A.) 88:1143-1147; Prezant et al. (1992) Hum. Mutat. 1:159-164; Ugozzoli et al. (1992) GATA 9:107-112; Nyren et al. (1993) Anal. Biochem. 208:171-175). These methods differ from GBA™ in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen et al. (1993) Amer. J. Hum. Genet. 52:46-59).

In one aspect the application provided for a panel of genetic markers selected from, but not limited to the genetic polymorphisms above. The panel comprises probes or primers that can be used to amplify and/or for determining the molecular structure of the polymorphisms identified above. The probes or primers can be attached or supported by a solid phase support such as, but not limited to a gene chip or microarray. The probes or primers can be detectably labeled. This aspect of the application is a means to identify the genotype of a patient sample for the genes of interest identified above. In one aspect, the methods of the application provided for a means of using the panel to identify or screen patient samples for the presence of the genetic marker identified herein. In one aspect, the various types of panels provided by the application include, but are not limited to, those described herein. In one aspect, the panel contains the above identified probes or primers as wells as other, probes or primers. In an alternative aspect, the panel includes one or more of the above noted probes or primers and others. In a further aspect, the panel consists only of the above-noted probes or primers.

In some embodiments of the application, probes are labeled with two fluorescent dye molecules to form so-called “molecular beacons” (Tyagi and Kramer (1996) Nat. Biotechnol. 14:303-8). Such molecular beacons signal binding to a complementary nucleic acid sequence through relief of intramolecular fluorescence quenching between dyes bound to opposing ends on an oligonucleotide probe. The use of molecular beacons for genotyping has been described (Kostrikis (1998) Science 279:1228-9) as has the use of multiple beacons simultaneously (Marras (1999) Genet. Anal. 14:151-6). A quenching molecule is useful with a particular fluorophore if it has sufficient spectral overlap to substantially inhibit fluorescence of the fluorophore when the two are held proximal to one another, such as in a molecular beacon, or when attached to the ends of an oligonucleotide probe from about 1 to about 25 nucleotides.

Labeled probes also can be used in conjunction with amplification of a polymorphism. (Holland et al. (1991) Proc. Natl. Acad. Sci. 88:7276-7280). U.S. Pat. No. 5,210,015 by Gelfand et al. describe fluorescence-based approaches to provide real time measurements of amplification products during PCR. Such approaches have either employed intercalating dyes (such as ethidium bromide) to indicate the amount of double-stranded DNA present, or they have employed probes containing fluorescence-quencher pairs (also referred to as the “Taq-Man” approach) where the probe is cleaved during amplification to release a fluorescent molecule whose concentration is proportional to the amount of double-stranded DNA present. During amplification, the probe is digested by the nuclease activity of a polymerase when hybridized to the target sequence to cause the fluorescent molecule to be separated from the quencher molecule, thereby causing fluorescence from the reporter molecule to appear. The Taq-Man approach uses a probe containing a reporter molecule-quencher molecule pair that specifically anneals to a region of a target polynucleotide containing the polymorphism.

Probes can be affixed to surfaces for use as “gene chips” or “microarray.” Such gene chips or microarrays can be used to detect genetic variations by a number of techniques known to one of skill in the art. In one technique, oligonucleotides are arrayed on a gene chip for determining the DNA sequence of a by the sequencing by hybridization approach, such as that outlined in U.S. Pat. Nos. 6,025,136 and 6,018,041. The probes of the application also can be used for fluorescent detection of a genetic sequence. Such techniques have been described, for example, in U.S. Pat. Nos. 5,968,740 and 5,858,659. A probe also can be affixed to an electrode surface for the electrochemical detection of nucleic acid sequences such as described by Kayem et al. U.S. Pat. No. 5,952,172 and by Kelley et al. (1999) Nucleic Acids Res. 27:4830-4837.

Various “gene chips” or “microarray” and similar technologies are known in the art. Examples of such include, but are not limited to LabCard (ACLARA Bio Sciences Inc.); GeneChip (Affymetrix, Inc); LabChip (Caliper Technologies Corp); a low-density array with electrochemical sensing (Clinical Micro Sensors); LabCD System (Gamera Bioscience Corp.); Omni Grid (Gene Machines); Q Array (Genetix Ltd.); a high-throughput, automated mass spectrometry systems with liquid-phase expression technology (Gene Trace Systems, Inc.); a thermal jet spotting system (Hewlett Packard Company); Hyseq HyChip (Hyseq, Inc.); BeadArray (Illumina, Inc., San Diego WO 99/67641 and WO 00/39587); GEM (Incyte Microarray Systems); a high-throughput microarraying system that can dispense from 12 to 64 spots onto multiple glass slides (Intelligent Bio-Instruments); Molecular Biology Workstation and NanoChip (Nanogen, Inc.); a microfluidic glass chip (Orchid biosciences, Inc.); surface tension array (ProtoGene, Palo Alto, Calif. U.S. Pat. Nos. 6,001,311; 5,985,551; and 5,474,796), BioChip Arrayer with four PiezoTip piezoelectric drop-on-demand tips (Packard Instruments, Inc.); FlexJet (Rosetta Inpharmatic, Inc.); MALDI-TOF mass spectrometer (Sequenome); ChipMaker 2 and ChipMaker 3 (TeleChem International, Inc.); and GenoSensor (Vysis, Inc.) as identified and described in Heller (2002) Annu Rev. Biomed. Eng. 4:129-153. Examples of “Gene chips” or a “microarray” are also described in US Patent Publ. Nos.: 2007-0111322, 2007-0099198, 2007-0084997, 2007-0059769 and 2007-0059765 and U.S. Pat. Nos. 7,138,506, 7,070,740, and 6,989,267.

In one aspect, “gene chips” or “microarrays” containing probes or primers for genes of the application alone or in combination are prepared. A suitable sample is obtained from the patient extraction of genomic DNA, RNA, or any combination thereof and amplified if necessary. The DNA or RNA sample is contacted to the gene chip or microarray panel under conditions suitable for hybridization of the gene(s) of interest to the probe(s) or primer(s) contained on the gene chip or microarray. The probes or primers may be detectably labeled thereby identifying the polymorphism in the gene(s) of interest. Alternatively, a chemical or biological reaction may be used to identify the probes or primers which hybridized with the DNA or RNA of the gene(s) of interest. The genotypes of the patient is then determined with the aid of the aforementioned apparatus and methods.

An allele may also be detected indirectly, e.g. by analyzing the protein product encoded by the DNA. For example, where the marker in question results in the translation of a mutant protein, the protein can be detected by any of a variety of protein detection methods. Such methods include immunodetection and biochemical tests, such as size fractionation, where the protein has a change in apparent molecular weight either through truncation, elongation, altered folding or altered post-translational modifications. Methods for measuring gene expression are also well known in the art and include, but are not limited to, immunological assays, nuclease protection assays, northern blots, in situ hybridization, reverse transcriptase Polymerase Chain Reaction (RT-PCR), Real-Time Polymerase Chain Reaction, expressed sequence tag (EST) sequencing, cDNA microarray hybridization or gene chip analysis, statistical analysis of microarrays (SAM), subtractive cloning, Serial Analysis of Gene Expression (SAGE), Massively Parallel Signature Sequencing (MPSS), and Sequencing-By-Synthesis (SBS). See for example, Carulli et al., (1998) J. Cell. Biochem. 72 (S30-31): 286-296; Galante et al., (2007) Bioinformatics, Advance Access (Feb. 3, 2007).

SAGE, MPSS, and SBS are non-array based assays that determine the expression level of genes by measuring the frequency of sequence tags derived from polyadenylated transcripts. SAGE allows for the analysis of overall gene expression patterns with digital analysis. SAGE does not require a preexisting clone and can used to identify and quantitate new genes as well as known genes. See for example, Velculescu et al., (1995) Science 270(5235):484-487; Velculescu (1997) Cell 88(2):243-251.

MPSS technology allows for analyses of the expression level of virtually all genes in a sample by counting the number of individual mRNA molecules produced from each gene. As with SAGE, MPSS does not require that genes be identified and characterized prior to conducting an experiment. MPSS has a sensitivity that allows for detection of a few molecules of mRNA per cell. See for example, Brenner et al. (2000) Nat. Biotechnol. 18:630-634; Reinartz et al., (2002) Brief Funct. Genomic Proteomic 1: 95-104.

SBS allows analysis of gene expression by determining the differential expression of gene products present in sample by detection of nucleotide incorporation during a primer-directed polymerase extension reaction.

SAGE, MPSS, and SBS allow for generation of datasets in a digital format that simplifies management and analysis of the data. The data generated from these analyses can be analyzed using publicly available databases such as Sage Genie (see, for example, Boon et al., 2002, PNAS 99:11287-92), SAGEmap (see, for example, Lash et al., 2000, Genome Res 10:1051-1060), and Automatic Correspondence of Tags and Genes (ACTG) (Galante (2007), supra). The data can also be analyzed using databases constructed using in house computers (see, for example, Blackshaw et al. 2004, PLoS Biol, 2:E247; Silva et al. 2004, Nucleic Acids Res 32:6104-6110)).

Moreover, it will be understood that any of the above methods for detecting alterations in a gene or gene product or polymorphic variants can be used to monitor the course of treatment or therapy.

The methods described herein may be performed, for example, by utilizing pre-packaged diagnostic kits, such as those described below, comprising at least one probe or primer nucleic acid described herein, which may be conveniently used, e.g., to determine whether an individual has or may have a greater or lower response to a particular treatment(s).

Diagnostic procedures can also be performed in situ directly upon samples from, such that no nucleic acid purification is necessary. Nucleic acid reagents can be used as probes and/or primers for such in situ procedures (see, for example, Nuovo (1992) “PCR IN SITU HYBRIDIZATION: PROTOCOLS AND APPLICATIONS”, Raven Press, NY).

In addition to methods that focus primarily on the detection of one nucleic acid sequence, profiles can also be assessed in such detection schemes. Fingerprint profiles can be generated, for example, by utilizing a differential display procedure, Northern analysis and/or RT-PCR.

### G. Nucleic Acid Molecules

In one aspect, the nucleic acid sequences of the gene's allelic variants, or portions thereof, can be the basis for probes or primers, e.g., in methods and compositions for determining and identifying the allele present at the gene of interest's locus, more particularly to identity the allelic variant of a polymorphic region(s). Thus, they can be used in the methods of the present application to determine which therapy is most likely to affect or not affect an individual's skin phenotypic attribute, such as to diagnose and prognose skin disease progression as well as select the most effective treatment among treatment options. In some embodiments, probes can be used to directly determine the genotype of the individual or can be used simultaneously with or subsequent to amplification.

In some embodiments, the methods of the present application can use nucleic acids isolated from vertebrates. In some embodiments, the vertebrate nucleic acids are nucleic acids isolated from a mammalian organism. In some embodiments, the nucleic acids used in the methods of the application are nucleic acids isolated from human.

Primers and probes for use in the methods of the present application are nucleic acids that hybridize to a nucleic acid sequence which is adjacent to the region of interest or which covers the region of interest and is extended. A primer or probe can be used alone in a detection method, or a can be used together with at least one other primer or probe in a detection method. Primers, and in some embodiments probes, can also be used to amplify at least a portion of a nucleic acid. In some embodiments, probes for use in the methods of the present application may be nucleic acids which hybridize to the region of interest and which are generally not extended further. However, probes for use in the kits of the present application may be an extendable nucleic acid, where appropriate. In some embodiments, probes may be further labeled, for example by nick translation, Klenow fill-in reaction, PCR, or other methods known in the art, including those described herein. For example, a probe is a nucleic acid which hybridizes to the polymorphic region of the gene of interest, and which by hybridization or absence of hybridization to the DNA of a subject will be indicative of the identity of the allelic variant of the polymorphic region of the gene of interest. Particular embodiments of probes and primers of the present application, their preparation, and/or labeling are described in Green and Sambrook (2012).

In some embodiments, primers and probes of the present application comprise a nucleotide sequence which comprises a region having a nucleotide sequence which hybridizes under stringent conditions to about 5 through about 100 consecutive nucleotides, more particularly about: 6, 8, 10, 12, 15, 20, 25, 30, 35, 40, 45, 50, 60, or 75 consecutive nucleotides of the gene of interest. Length of the primer or probe used will depend, in part, on the nature of the assay used and the hybridization conditions employed.

The term “hybridization”, as used herein, refers generally to the ability of nucleic acid molecules to join via complementary base strand pairing. Such hybridization may occur when nucleic acid molecules are contacted under appropriate conditions and/or circumstances. As used herein, two nucleic acid molecules are said to be capable of specifically hybridizing to one another if the two molecules are capable of forming an anti-parallel, double-stranded nucleic acid structure. A nucleic acid molecule is said to be the “complement” of another nucleic acid molecule if they exhibit complete complementarity. As used herein, nucleic acid molecules are said to exhibit “complete complementarity” when every nucleotide of one of the molecules is complementary to its base pairing partner nucleotide of the other. Two molecules are said to be “minimally complementary” if they can hybridize to one another with sufficient stability to permit them to remain annealed to one another under at least conventional “low-stringency” conditions. In some instances, the molecules are said to be “complementary” if they can hybridize to one another with sufficient stability to permit them to remain annealed to one another under conventional “high-stringency” conditions. Nucleic acid molecules that hybridize to other nucleic acid molecules, e.g., at least under low stringency conditions are said to be “hybridizable cognates” of the other nucleic acid molecules. Conventional stringency conditions are described by Sambrook et al., Molecular Cloning, A Laboratory Handbook, Cold Spring Harbor Laboratory Press, 1989), and by Haymes et al. In: Nucleic Acid Hybridization, A Practical Approach, IRL Press, Washington, D.C. (1985). Departures from complete complementarity are therefore permissible, as long as such departures do not completely preclude the capacity of the molecules to form a double-stranded structure. Thus, in order for a nucleic acid molecule or fragment thereof of the present disclosure to serve as a primer or probe it needs only have sufficient complementarity in sequence to be able to form a stable double-stranded structure under the particular solvent and salt concentrations employed.

Appropriate stringency conditions which promote DNA hybridization include, for example, 6.0× sodium chloride/sodium citrate (SSC) at about 45° C., followed by a wash of 2.0×SSC at about 50° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed. These conditions are known to those skilled in the art, or can be found in Current Protocols in Molecular Biology, John Wiley & Sons, N.Y. (1989), 6.3.1-6.3.6. For example, low stringency conditions may be used to select nucleic acid sequences with lower sequence identities to a target nucleic acid sequence. One may wish to employ conditions such as about 0.15 M to about 0.9 M sodium chloride, at temperatures ranging from about 20° C. to about 55° C. High stringency conditions may be used to select for nucleic acid sequences with higher degrees of identity to the disclosed nucleic acid sequences (Sambrook et al., 1989, supra). In some embodiments of the present disclosure, high stringency conditions involve nucleic acid hybridization in about 2×SSC to about 10×SSC (diluted from a 20×SSC stock solution containing 3 M sodium chloride and 0.3 M sodium citrate, pH 7.0 in distilled water), about 2.5x to about 5×Denhardt's solution (diluted from a 50x stock solution containing 1% (w/v) bovine serum albumin, 1% (w/v) ficoll, and 1% (w/v) polyvinylpyrrolidone in distilled water), about 10 mg/mL to about 100 mg/mL fish sperm DNA, and about 0.02% (w/v) to about 0.1% (w/v) SDS, with an incubation at about 50° C. to about 70° C. for several hours to overnight. High stringency conditions are preferably provided by 6×SSC, 5×Denhardt's solution, 100 mg/mL sheared and denatured salmon sperm DNA, and 0.1% (w/v) SDS, with incubation at 55×C for several hours. Hybridization is generally followed by several wash steps. The wash compositions generally comprise 0.5×SSC to about 10×SSC, and 0.01% (w/v) to about 0.5% (w/v) SDS with a 15-min incubation at about 20° C. to about 70° C. Preferably, the nucleic acid segments remain hybridized after washing at least one time in 0.1×SSC at 65° C. In some instances, very high stringency conditions may be used to select for nucleic acid sequences with much higher degrees of identity to the disclosed nucleic acid sequences. Very high stringency conditions are defined as prehybridization and hybridization at 42° C. in 5×SSPE, 0.3% SDS, 200 μg/mL sheared and denatured salmon sperm DNA, and 50% formamide and washing three times each for 15 minutes using 2×SSC, 0.2% SDS at 70° C.

In some embodiments disclosed herein, primers (and in some embodiments probes) of the present application can be complementary to nucleotide sequences located close to each other or further apart, depending on the use of the amplified DNA. For example, primers or probes can be chosen such that they amplify DNA fragments of at least about 10 nucleotides or as much as several kilobases. Preferably, the primers or probes of the present application will hybridize selectively to nucleotide sequences located about 150 to about 350 nucleotides apart.

For amplifying at least a portion of a nucleic acid, a forward primer (or probe; i.e., 5′ primer) and a reverse primer (or probe; i.e., 3′ primer) will preferably be used. Forward and reverse primers (or probes) hybridize to complementary strands of a double stranded nucleic acid, such that upon extension from each primer, a double stranded nucleic acid is amplified.

Yet other preferred primers of the present application are nucleic acids that are capable of selectively hybridizing to an allelic variant of a polymorphic region of the gene of interest. Thus, such primers can be specific for allelic variants of the gene of interest sequence, so long as they have a nucleotide sequence that is capable of hybridizing to the gene of interest.

The probe or primer may further comprises a label attached thereto, which, e.g., is capable of being detected, e.g. the label group is selected from amongst radioisotopes, fluorescent compounds, enzymes, and enzyme co-factors.

Additionally, the isolated nucleic acids used as probes or primers may be modified to become more stable. Exemplary nucleic acid molecules that are modified include phosphoramidate, phosphothioate, and methylphosphonate analogs of DNA (see also U.S. Pat. Nos. 5,176,996; 5,264,564 and 5,256,775).

The nucleic acids used in the methods of the application can also be modified at the base moiety, sugar moiety, or phosphate backbone, for example, to improve stability of the molecule. The nucleic acids, e.g., probes or primers, may include other appended groups such as peptides (e.g., for targeting host cell receptors in vivo), or agents facilitating transport across the cell membrane. See, e.g., Letsinger et al., (1989) Proc. Natl. Acad. Sci. U.S.A. 86:6553-6556; Lemaitre et al., (1987) Proc. Natl. Acad. Sci. 84:648-652; and PCT Publication No. WO 88/09810, published Dec. 15, 1988), hybridization-triggered cleavage agents, (see, e.g., Krol et al., (1988) BioTechniques 6:958-976) or intercalating agents (see, e.g., Zon (1988) Pharm. Res. 5:539-549. To this end, the nucleic acid used in the methods of the present application may be conjugated to another molecule, e.g., a peptide, hybridization triggered cross-linking agent, transport agent, hybridization-triggered cleavage agent, etc.

The isolated nucleic acids used in the methods of the present application can also comprise at least one modified sugar moiety selected from the group including but not limited to arabinose, 2-fluoroarabinose, xylulose, and hexose or, alternatively, comprise at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

The nucleic acids, or fragments thereof, to be used in the methods of the present application can be prepared according to methods known in the art and described, e.g., in Sambrook and Russel (2001) supra. For example, discrete fragments of the DNA can be prepared and cloned using restriction enzymes. Alternatively, discrete fragments can be prepared using the Polymerase Chain Reaction (PCR) using primers having an appropriate sequence under the manufacturer's conditions, (described above).

Oligonucleotides can be synthesized by standard methods known in the art, e.g. by use of an automated DNA synthesizer (such as are commercially available from Biosearch, Applied Biosystems, etc.). As examples, phosphorothioate oligonucleotides can be synthesized by the method of Stein et al. (1988) Nucl. Acids Res. 16:3209, methylphosphonate oligonucleotides can be prepared by use of controlled pore glass polymer supports. Sarin et al. (1988) Proc. Natl. Acad. Sci. U.S.A. 85:7448-7451.

### Other Uses for the Nucleic Acids of the Application

The identification of the allele of the gene of interest can also be useful for identifying an individual among other individuals from the same species. For example, DNA sequences can be used as a fingerprint for detection of different individuals within the same species. Thompson and Thompson, Eds., (1991) GENETICS IN MEDICINE, W B Saunders Co., Philadelphia, Pa. This is useful, e.g., in forensic studies.

### H. Kits for Assessing Skin Conditions

As set forth herein, some embodiments relate to methods for determining the likelihood of an individual to exhibit one or more (or a plurality of) skin phenotypic attributes and/or for identifying a skin care regimen for an individual. Accordingly, some embodiments disclosed herein relate to kits for assessing the skin condition (e.g., skin health) of an individual. In some embodiments, the kits are provided for determining the likelihood of an individual to exhibit one or more (or a plurality of) skin phenotypic attributes. Some embodiments disclosed herein relate to kits for identifying a skin care regimen for an individual. The kits according to this aspect of the disclosure typically contain reagents for performing one or more of the methods described herein, including one or more of probes, primers, oligonucleotides, antibodies, salts, enzymes, buffers, etc., and optionally instructions for using the kits. The reagents used in certain embodiments of the methods described herein are further indicated below. Additional reagents useful for performing those methods using a variety of alternative sample preparations and genetic variation detection methods or chemistries are apparent to the skilled artisan upon reviewing the disclosure.

The presently disclosed kits include reagents that permit their user to detect occurrence of one or more genetic variations in at least three (or at least four, six, eight, ten, twelve, or fifteen or more) biomarkers disclosed herein. In some embodiments, the disclosed kits include reagents that permit their user to detect occurrence of one or more genetic variations in at least twenty (or at least thirty, forty, fifty, sixty, seventy, or more) biomarkers disclosed herein. In some embodiments, the kits of the present application include reagents that permit their user to detect occurrence of one or more genetic variations associated with each member of a first set and a second set of preselected biomarkers of an individual, wherein each member of the first set of biomarkers is genetically associated with one or more skin nutritional conditions and each member of the second set of biomarkers is genetically associated with one or more (or a plurality of) skin phenotypic attributes.

In some embodiments, the kits of the present application include a plurality of molecular probes, at least some of which are specific to one or more skin disorder-associated genetic variation that are genetically linked with one of the biomarkers and/or genes (e.g., one of the biomarkers and/or genes identified herein as being of particular relevance for skin health). In some embodiments, at least some of the molecular probes are specific to genetic variations of a polymorphic region present in the gene of interest or the expression level of a gene of interest. In some embodiments, the methods use probes and/or primers comprising nucleotide sequences which are complementary to the polymorphic region of the gene of interest. Accordingly, some embodiments provide kits for performing these methods as well as instructions for carrying out the methods of this application such as collecting biological samples and/or performing the selection/identification, and/or analyzing the results, and/or administration of an effective skin care regimen described above.

In some embodiments, the kits contain one of more of the compositions and reagents described above and instructions for use. In one exemplary embodiment, kits are provided for acquiring knowledge of the occurrence of one or more genetic variations associated with each member of a first set and a second set of preselected biomarkers in the individual, wherein each member of the first set of biomarkers is genetically associated with one or more skin nutritional conditions and each member of the second set of biomarkers is genetically associated with one or more (or a plurality of) skin phenotypic attributes. Oligonucleotides “specific for” a genetic variation bind either to the polymorphic region of the target biomarker and/or gene, or bind adjacent to the polymorphic region of the corresponding locus. For oligonucleotides that are to be used as primers (which may, in some embodiments, include probes) for amplification, primers are adjacent if they are sufficiently close to be used to produce a polynucleotide comprising the polymorphic region. In some embodiments, oligonucleotides are adjacent if they bind within about 1-2 kb, and preferably less than 1 kb from the genetic variation. One of skill in the art will immediately appreciate that specific oligonucleotides are capable of hybridizing to a sequence, and under suitable conditions will not bind to a sequence sufficiently differing by a single nucleotide.

In some embodiments, the kits disclosed herein can comprise at least one probe or primer which is capable of specifically hybridizing to the polymorphic region of the gene of interest and instructions for use. The kits preferably comprise at least one of the above described molecular probes such as, for example, oligonucleotides. Preferred kits for amplifying at least a portion of the gene of interest comprise two primers and two probes, at least one of the probes is capable of binding to the target genetic variation. Such kits are suitable for detection of genotype by, for example, fluorescence detection, by electrochemical detection, or by other detection methodologies known in the art.

In some embodiments, molecular probes such as oligonucleotides or antibodies, whether used as probes or primers, contained in a kit can be detectably labeled. Labels can be detected either directly, for example for fluorescent labels, or indirectly. Indirect detection can include any detection method known to one of skill in the art, including biotin-avidin interactions, antibody binding and the like. Fluorescently labeled oligonucleotides also can contain a quenching molecule.

Molecular probes can be immobilized to a surface. Accordingly, in some embodiments, at least some of the specific molecular probes can be attached to a surface in order to facilitate handling of the molecular probes. The molecular probes can be linked with a plurality of surfaces (e.g., molecular probes specific to a particular genetic variation being attached to a particle discrete from another particle to which molecular probes for another genetic variation are attached), or they can be attached to discrete regions of a single surface (e.g., a glass or silicon surface having molecular probes attached at defined locations thereon, as in the GENECHIP™ device of Affymetrix, Inc.). Coupling/annealing between individual molecular probes and the genetic variations corresponding thereto can be detected using standard methods. In some embodiments, the kits can also comprise molecular probes that are useful as molecular beacon probes or as extendable primers. In some embodiments, the preferred surface is silica or glass. In another embodiment, the surface is a metal electrode.

Yet other kits of the application, in accordance with some embodiments, comprise at least one reagent necessary to perform the analytical assay. For example, the kits can comprise an enzyme. Alternatively or in addition, the kits can comprise a buffer or any other necessary reagents.

In some embodiments, the kits of the present application can further comprise a biological sample collection kit or apparatus such as, for example, a sample collection means, including, but not limited to a buccal swab for collecting saliva and/or epithelial cells also in saliva, storage means for storing the collected sample, and for shipment. In some embodiments, the kits of the present application can further comprise a biological sample collection kit or apparatus such as those described in U.S. Pat. Nos. 8,617,487 and 8,932,539; and co-pending U.S. patent application Ser. No. 14/717,997. Advantageously, DNA collected using the kits or apparatus can be stored or archived, and subjected to additional testing as previously unknown skin health-associated genetic variations are discovered in the biomarkers and/or genes disclosed herein, or as the significance of previously unappreciated genetic variations is realized.

Conditions for incubating a molecular probe with a test sample depend on the format employed in the assay, the detection methods used, and the type and nature of the molecular probe used in the assay. One skilled in the art will recognize that a number of the commonly available hybridization, amplification or immunological assay formats can be adapted to employ the molecular probes for use in the present application.

In some embodiments, the kits according to the present disclosure further comprise a CD, or CD-ROM with instructions on how to collect sample, ship sample, and means to interpret genotypic information retrieved from the sample DNA and/or protein, and translating the information into therapeutic/dietary or lifestyle recommendation. As discussed in greater detail below, information data of an individual's genetic profile can be stored, transmitted and displayed via computer networks and the internet. The therapeutic/dietary and lifestyle recommendations can include, but not limited to, those described in the present disclosure.

Examples of such assays can be found in Chard (1986) AN INTRODUCTION TO RADIOIMMUNOASSAY AND RELATED TECHNIQUES, Elsevier Science Publishers, Amsterdam, The Netherlands; Bullock et al. TECHNIQUES IN IMMUNOCYTOCHEMISTRY Academic Press, Orlando, Fla. Vol. 1 (1982), Vol. 2 (1983), Vol. 3 (1985); Tijssen, PRACTICE AND THEORY OF IMMUNOASSAYS: LABORATORY TECHNIQUES IN BIOCHEMISTRY AND MOLECULAR BIOLOGY, Elsevier Science Publishers, Amsterdam, The Netherlands (1985).

Biological samples suitable for use in the kits disclosed herein can include nucleic acid extracts, cells, protein or membrane extracts of cells, or biological fluids such as sputum, blood, serum, plasma, or urine. The biological sample used in the above-described methods will vary based on the assay format, nature of the detection methods and the tissues, cells or extracts used as the biological sample to be assayed. Methods for preparing nucleic acid extracts, protein extracts or extracts of cells are known in the art and can be readily adapted in order to obtain a sample which is compatible with the detection method utilized.

In some embodiments, the kits disclosed herein can include all or some of the positive controls, negative controls, reagents, primers, sequencing markers, oligonucleotide probes and antibodies described herein for determining the individual's genetic variation (i.e. genotype) in the polymorphic region or the expression levels of the genes of interest.

As amenable, the above-suggested kit components may be packaged in a manner customary for use by those of skill in the art. For example, these suggested kit components may be provided in solution or as a liquid dispersion or the like.

### I. Non-Limiting Computer Embodiments

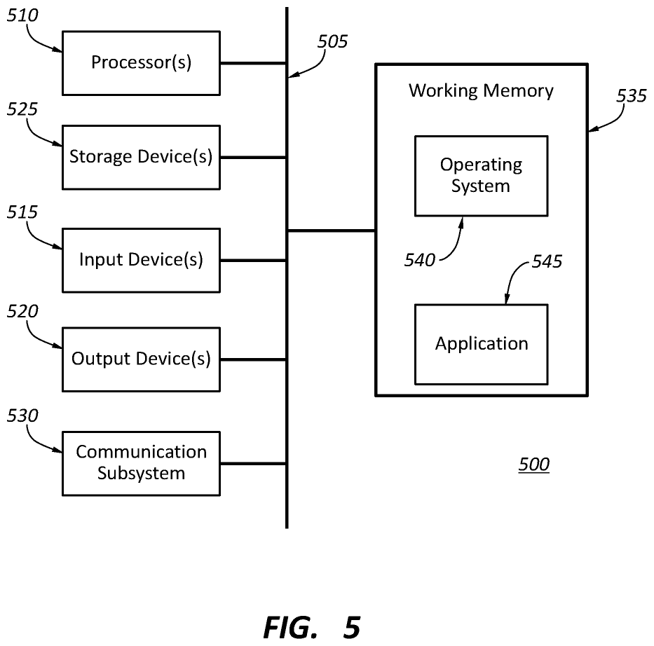

FIG. 5 provides a schematic illustration of one embodiment of a computer system 500 that can perform the methods of the application, as described herein. It should be noted that FIG. 5 is meant only to provide a generalized illustration of various components, any or all of which may be utilized as appropriate. FIG. 5, therefore, broadly illustrates how individual system elements may be implemented in a relatively separated or relatively more integrated manner.

The term “computer system” as used herein, refers to any conventional system including a processor, a main memory and a static memory, which are coupled by bus. In some embodiments, the computer system can further include a video display unit (e.g., a liquid crystal display (LCD) or cathode ray tube (CRT)) on which a user interface can be displayed). In some embodiments, the computer system can also include one or more of the followings: an alpha-numeric input device (e.g., a keyboard), a cursor control device (e.g., a mouse), a disk drive unit, a signal generation device (e.g., a speaker) and a network interface device medium. In some embodiments, the disk drive unit includes a computer-readable medium on which software can be stored. In some embodiments, the software can also reside, completely or partially, within the main memory and/or within the processor. In some embodiments, the software can also be transmitted or received via the network interface device. The term “computer-readable medium” is used herein to include any medium which is capable of storing or encoding a sequence of instructions for performing the methods described herein and can include, but not limited to, optical and/or magnetic storage devices and/or disks, and carrier wave signals.

The computer system 500 is shown comprising hardware elements that can be electrically coupled via a bus 505 (or may otherwise be in communication, as appropriate). The hardware elements can include one or more processors 510, including without limitation, one or more general purpose processors and/or one or more special purpose processors (such as digital signal processing chips, graphics acceleration chips, and/or the like); one or more input devices 515, which can include without limitation a mouse, a keyboard and/or the like; and one or more output devices 520, which can include without limitation a display device, a printer and/or the like.

The computer system 500 may further include (and/or be in communication with) one or more storage devices 525, which can comprise, without limitation, local and/or network accessible storage and/or can include, without limitation, a disk drive, a drive array, an optical storage device, a solid state storage device such as a random access memory (“RAM”) and/or a read-only memory (“ROM”), which can be programmable, flash updateable and/or the like. The computer system 500 might also include a communications subsystem 530, which can include without limitation a modem, a network card (wireless or wired), an infrared communication device, a wireless communication device and/or chipset (such as a Bluetooth™ device, an 802.11 device, a WiFi device, a WiMax device, cellular communication facilities, etc.), and/or the like. The communications subsystem 530 may permit data to be exchanged with a network (such as the network described below, to name one example), and/or any other devices described herein. In many embodiments, the computer system 500 will further comprise a working memory 535, which can include a RAM or ROM device, as described above.

The computer system 500 also can comprise software elements, shown as being currently located within the working memory 535, including an operating system 540 and/or other code, such as one or more application programs 545, which may comprise computer programs of the application, and/or may be designed to implement methods of the application and/or configure systems of the application, as described herein. Merely by way of example, one or more procedures described with respect to the method(s) discussed above might be implemented as code and/or instructions executable by a computer (and/or a processor within a computer). A set of these instructions and/or codes might be stored on a computer-readable storage medium, such as the storage device(s) 525 described above. In some cases, the storage medium might be incorporated within a computer system, such as the system 500. In other embodiments, the storage medium might be separate from a computer system (i.e., a removable medium, such as a compact disc, etc.), and is provided in an installation package, such that the storage medium can be used to program a general-purpose computer with the instructions/code stored therein. These instructions might take the form of executable code, which is executable by the computer system 500 and/or might take the form of source and/or installable code, which, upon compilation and/or installation on the computer system 500 (e.g., using any of a variety of generally available compilers, installation programs, compression/decompression utilities, etc.), then takes the form of executable code.

It will be apparent to those skilled in the art that substantial variations may be made in accordance with specific requirements. For example, customized hardware might also be used, and/or particular elements might be implemented in hardware, software (including portable software, such as applets, etc.), or both. Further, connection to other computing devices such as network input/output devices may be employed.

In one aspect, the application employs a computer system (such as the computer system 500) to perform methods of the application. According to a set of embodiments, some or all of the procedures of such methods are performed by the computer system 500 in response to processor 510 executing one or more sequences of one or more instructions (which might be incorporated into the operating system 540 and/or other code, such as an application program 545) contained in the working memory 535. Such instructions may be read into the working memory 535 from another machine-readable medium, such as one or more of the storage device(s) 525. Merely by way of example, execution of the sequences of instructions contained in the working memory 535 might cause the processor(s) 510 to perform one or more procedures of the methods described herein.

Some embodiments disclosed herein relate to a computer readable medium. The terms “computer readable medium” and “machine-readable medium,” as used herein, refer to any medium that participates in providing data that causes a machine to operate in a specific fashion. In an embodiment implemented using the computer system 500, various machine-readable media might be involved in providing instructions/code to processor(s) 510 for execution and/or might be used to store and/or carry such instructions/code (e.g., as signals). In many implementations, a computer-readable medium is a physical and/or tangible storage medium. Such a medium may take many forms, including but not limited to, non-volatile media, volatile media, and transmission media. Non-volatile media includes, for example, optical or magnetic disks, such as the storage device(s) 525. Volatile media includes, without limitation, dynamic memory, such as the working memory 535. Transmission media includes coaxial cables, copper wire and fiber optics, including the wires that comprise the bus 505, as well as the various components of the communications subsystem 530 (and/or the media by which the communications subsystem 530 provides communication with other devices). Hence, transmission media can also take the form of waves (including without limitation radio, acoustic and/or light waves, such as those generated during radio wave and infrared data communications).

Common forms of physical and/or tangible computer-readable media include, for example, a floppy disk, a flexible disk, a hard disk, magnetic tape, or any other magnetic medium, a CD-ROM, any other optical medium, punchcards, papertape, any other physical medium with patterns of holes, a RAM, a PROM, an EPROM, a FLASH-EPROM, any other memory chip or cartridge, a carrier wave as described hereinafter, or any other medium from which a computer can read instructions and/or code.

Various forms of machine-readable media may be involved in carrying one or more sequences of one or more instructions to the processor(s) 510 for execution. Merely by way of example, the instructions may initially be carried on a magnetic disk and/or optical disc of a remote computer. A remote computer might load the instructions into its dynamic memory and send the instructions as signals over a transmission medium to be received and/or executed by the computer system 500. These signals, which might be in the form of electromagnetic signals, acoustic signals, optical signals and/or the like, are all examples of carrier waves on which instructions can be encoded, in accordance with various embodiments of the application.

The communications subsystem 530 (and/or components thereof) generally will receive the signals, and the bus 505 then might carry the signals (and/or the data, instructions, etc., carried by the signals) to the working memory 535, from which the processor(s) 510 retrieves and executes the instructions. The instructions received by the working memory 535 may optionally be stored on a storage device 525 either before or after execution by the processor(s) 510.

In some embodiments, the computer readable medium disclosed herein is a non-transitory computer readable medium containing executable instructions that when executed cause a processor to perform operations including (a) receiving an individual's personalized genetic profile of a first set and a second set of biomarkers in the individual, wherein each member of the first set of biomarkers is genetically associated with one or more skin nutritional conditions and each member of the second set of biomarkers is genetically associated with one or more skin phenotypic attributes; (b) assigning, based at least in part on the personalized biomarker profile, a relative biomarker score to each of the one or more skin nutritional conditions and the one or more skin phenotypic attributes, each biomarker score indicating whether the individual has an enhanced, diminished, or average risk of the likelihood of exhibiting the skin phenotypic attributes or the one or more skin nutritional conditions; and (c) outputting a personalized skin care regimen for the individual based upon the assigned risk scores.

In some embodiments, the personalized biomarker profile generated for the individual can be compared to the multivariable scoring matrix to obtain a relative marker score, wherein the multivariable scoring matrix correlates patterns of genetic variations with probabilities of exhibiting phenotypic attributes. The multivariable scoring matrix correlates patterns of genetic variations with probabilities of exhibiting phenotypic attributes, based on scoring matrix vectors that can include one or more descriptors such as, for example, family history, general medical physiological measures or values (such as, but not limited to, cholesterol levels, blood pressure, heart rate, growth hormone levels, triglyceride levels, red blood cells, bone density, CD scan results, etc.), mRNA expression profiles, methylation profiles, protein expression profiles, enzyme activity, antibody load, nucleotide sequence homology, relative synteny among the preselected biomarkers, ontological relevance, quality of supporting research, degree of phenotypic significance, and the like. The multivariable matrix correlates patterns of genetic variations with probabilities of exhibiting phenotypic attributes, as described hereinabove. Then it is determined whether the probability score indicates that the individual would have an enhanced, diminished, or average likelihood of exhibiting one or more phenotypic attributes.

It will be appreciated that in some embodiments, the personalized genotype profile and associated likelihoods for exhibiting one or more phenotypic attributes may be expressed in a report. The report may, in some embodiments, be generated at a computing system and may additionally be displayed at the computing system at one or more output devices, including, for example, a display. A user interface of the display may be used by an individual to access and view the report thereon. In one or more embodiments, the user interface displaying the report provides a technical benefit. The data may be arranged in a summarized format, similar to that provided in TABLE 2, wherein the relevant information is readily available and viewable by a user. In providing the data in an accessible format at the user interface, it precludes or reduces the amount and frequency of user-implemented processing interruptions, such as, for example, movement and signaling of an input device about the display. Such searching and “clicking” at user interfaces can be resource intensive. Any processes running on the computer system (e.g., at the one or more processors) are interrupted by the user interaction such that the user interactions are prioritized over the previously processed information. Further, user interactions are often erratic in both duration and interval, which places a reiterative stress on the processing. By increasing the efficiency by which the data are reported and displayed at the user interface, less user interaction is needed, and consequently, the system becomes more efficient from a computing perspective.

Additionally, the algorithms and weighting schemes provided herein provide technical advantages when provided and/or implemented at a computing system. For example, by preselecting a set of genetic variations, the computing system is spared from accessing all data points; instead, it is streamlined and accessing only those data points necessary and relevant. On the other hand, current methods and systems lack such trimming steps. The present invention may additionally provide a technical advantage in that a trimming and/or filtering step may occur not only at the selection of genetic variations, but additionally at reporting thresholds that may be set by the user or the computing system. That is, a reporting threshold may be set that prevents reporting of a phenotypic attribute or genetic variations associated therewith if the values and/or weights associated with one or both of the foregoing are below a predetermined mark/threshold. This type of threshold may be implemented to, for example, avoid reporting data that are not significant or at least prevents reporting data that may mislead or potentially be erroneous. In such a way, the trimming/filtering of lower weights/values may act as a quality control step while simultaneously acting to improve the processing power of computers implementing the disclosed methods. This may be particularly exacerbated in cloud computing environments where a reduction in computing (e.g., processing or memory) that results from the aforementioned trimming/filtering allows for either an increase in the number of clients that can be serviced or it may alternatively allow for a reduction in system requirements. These advantages, together with those described herein, are not all of the advantages offered by the current application and claimed embodiments but are exemplary only.

The comparison with the multivariable scoring matrix can be done manually or, preferably, by employing a suitable computer software instantiation in which the multivariable scoring matrix is algorithmically constructed and manipulated via a programming language, for example, but not limited to, Java, Perl, C, or C++. Further, in some embodiments of the present application, the results of the genetic test and outcomes could be analyzed by a machine learning artificial intelligence, such as the IBM Watson system, in order to find more personal relationships and action items for the patient.

Based on this comparison it can be determined whether the relative biomarker score indicates an enhanced, diminished, or average likelihood of exhibiting one or more phenotypic attributes, relative to a reference population, e.g., the general population of a chosen geographical area, or another chosen subpopulation thereof in terms of ethnicity, gender, age, or other identifying feature of interest.

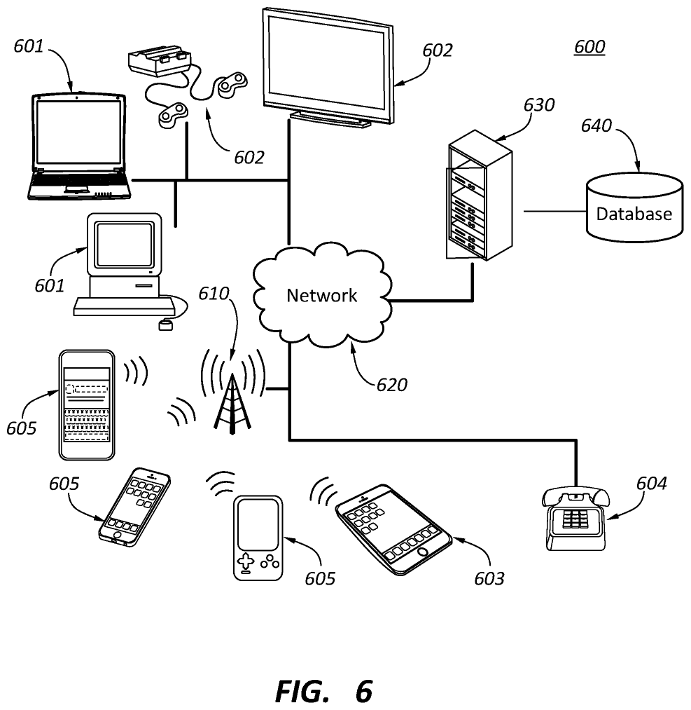

Merely by way of example, FIG. 6 illustrates a schematic diagram of devices to access and implement the application system 600. The system 600 can include one or more user computers 601. The user computers 601 can be general-purpose personal computers (including, merely by way of example, personal computers and/or laptop computers running any appropriate flavor of Microsoft Corp.'s Windows™ and/or Apple Corp.'s Macintosh™ operating systems) and/or workstation computers running any of a variety of commercially available UNIX™ or UNIX-like operating systems. These user computers 601 can also have any of a variety of applications, including one or more applications configured to perform methods of the application, as well as one or more office applications, database client and/or server applications, and web browser applications. Alternatively, the user computers 601 can be any other electronic device, such as a thin-client computer, media computing platforms 602 (e.g., gaming platforms, or cable and satellite set top boxes with navigation and recording capabilities), handheld computing devices (e.g., PDAs, tablets or handheld gaming platforms) 603, conventional land lines 604 (wired and wireless), mobile (e.g., cell or smart) phones 605 or tablets, or any other type of portable communication or computing platform (e.g., vehicle navigation systems), capable of communicating via a network (e.g., the network 620 described below) and/or displaying and navigating web pages or other types of electronic documents. Although the exemplary system 600 is shown with a user computer 601, any number of user computers can be supported.

Certain embodiments of the application operate in a networked environment, which can include a network 620. The network 620 can be any type of network familiar to those skilled in the art that can support data communications using any of a variety of commercially available protocols, including without limitation TCP/IP, SNA, IPX, AppleTalk, and the like. Merely by way of example, the network 620 can be a local area network (“LAN”), including without limitation an Ethernet network, a Token-Ring network and/or the like; a wide-area network (WAN); a virtual network, including without limitation a virtual private network (“VPN”); the Internet; an intranet; an extranet; a public switched telephone network (“PSTN”); an infrared network; a wireless network 610, including without limitation a network operating under any of the IEEE 802.11 suite of protocols, the Bluetooth™ protocol known in the art, and/or any other wireless protocol 610; and/or any combination of these and/or other networks.

Embodiments of the application can include one or more server computers 630. Each of the server computers 630 may be configured with an operating system, including without limitation any of those discussed above, as well as any commercially (or freely) available server operating systems. Each of the servers 630 may also be running one or more applications, which can be configured to provide services to one or more clients and/or other servers.

Merely by way of example, one of the servers 630 may be a web server, which can be used, merely by way of example, to process requests for web pages or other electronic documents from user computers 601. The web server can also run a variety of server applications, including HTTP servers, FTP servers, CGI servers, database servers, Java™ servers, and the like. In some embodiments of the application, the web server may be configured to serve web pages that can be operated within a web browser on one or more of the user computers 601 to perform methods of the application.

The server computers 630, in some embodiments, might include one or more application servers, which can include one or more applications accessible by a client running on one or more of the client computers and/or other servers. Merely by way of example, the server(s) 630 can be one or more general purpose computers capable of executing programs or scripts in response to the user computers and/or other servers, including without limitation web applications (which might, in some cases, be configured to perform methods of the application). Merely by way of example, a web application can be implemented as one or more scripts or programs written in any suitable programming language, such as Java™, C, C.T.M. or C++, and/or any scripting language, such as Perl, Python, or TCL, as well as combinations of any programming/scripting languages. The application server(s) can also include database servers, including without limitation those commercially available from Oracle™, Microsoft™ Sybase™ IBM™ and the like, which can process requests from clients (including, depending on the configuration, database clients, API clients, web browsers, etc.) running on a user computer and/or another server. In some embodiments, an application server can create web pages dynamically for displaying the information in accordance with embodiments of the application. Data provided by an application server may be formatted as web pages (comprising HTML, Javascript, etc., for example) and/or may be forwarded to a user computer via a web server (as described above, for example). Similarly, a web server might receive web page requests and/or input data from a user computer and/or forward the web page requests and/or input data to an application server. In some cases a web server may be integrated with an application server.

In accordance with further embodiments, one or more servers 630 can function as a file server and/or can include one or more of the files (e.g., application code, data files, etc.) necessary to implement methods of the application incorporated by an application running on a user computer and/or another server. Alternatively, as those skilled in the art will appreciate, a file server can include all necessary files, allowing such an application to be invoked remotely by a user computer and/or server. It should be noted that the functions described with respect to various servers herein (e.g., application server, database server, web server, file server, etc.) can be performed by a single server and/or a plurality of specialized servers, depending on implementation-specific needs and parameters.

In certain embodiments, the system can include one or more databases 640. The location of the database(s) 640 is discretionary. Merely by way of example, a database might reside on a storage medium local to (and/or resident in) a server (and/or a user computer). Alternatively, a database can be remote from any or all of the computers, so long as the database can be in communication (e.g., via the network) with one or more of these. In a particular set of embodiments, a database can reside in a storage-area network (“SAN”) familiar to those skilled in the art. (Likewise, any necessary files for performing the functions attributed to the computers can be stored locally on the respective computer and/or remotely, as appropriate.) In one set of embodiments, the database can be a relational database, such as an Oracle™ database, that is adapted to store, update, and retrieve data in response to SQL-formatted commands. The database might be controlled and/or maintained by a database server, as described above, for example.

While the application has been particularly shown and described with reference to specific embodiments thereof, it will be understood by those skilled in the art that changes in the form and details of the disclosed embodiments may be made without departing from the spirit or scope of the application. For example, embodiments have been described herein with reference to the use of conventional landlines and cellular phones. Additionally, the various embodiments of the application as described may be implemented in the form of software running on a general purpose computer, in the form of a specialized hardware, or combination of software and hardware. It will be understood, however, that the application is not so limited. That is, embodiments are contemplated in which a much wider diversity of communication devices may be employed in various combinations to effect redemption.

The present application can be performed without undue experimentation using, unless otherwise indicated, conventional systems and techniques of molecular biology, microbiology, virology, recombinant DNA technology, peptide synthesis in solution, solid phase peptide synthesis, histology and immunology. Detailed information relating to such systems, techniques, and procedures can be found, for example, in the following texts that are incorporated by reference.
(i) Green M R, Sambrook J, Molecular Cloning: A Laboratory Manual, Cold Spring Harbor Laboratories Press, New York, Fourth Edition (2012), whole of Vols I, II, and III;
(ii) DNA Cloning: A Practical Approach, Vols. I-IV (D. M. Glover, ed., 1995), Oxford University Press, whole of text;
(iii) Oligonucleotide Synthesis: Methods and Application (P Herdewijn, ed., 2010) Humana Press, Oxford, whole of text;
(iv) Nucleic Acid Hybridization: A Practical Approach (B. D. Hames & S. J. Higgins, eds., 1985) IRL Press, Oxford, whole of text;
(v) van Pelt-Verkuil, E, van Belkum, A, Hays, J P. Principles and Technical Aspects of PCR Amplification (2010) Springer, whole of text;
(vi) Perbal, B., A Practical Guide to Molecular Cloning, 3rd Ed. (2008);
(vii) Gene Synthesis: Methods and Protocols (J Peccoud, ed. 2012) Humana Press, whole of text;
(viii) PCR Primer Design (Methods in Molecular Biology). (A Yuryev. ed., 2010), Humana Press, Oxford, whole of text.

Throughout this disclosure, various publications, patents and published patent specifications are referenced by an identifying citation. All publications and patent applications mentioned in this specification are herein incorporated by reference to the same extent as if each individual publication or patent application was specifically and individually indicated to be incorporated by reference.

No admission is made that any reference cited herein constitutes prior art. The discussion of the references states what their authors assert, and the applicants reserve the right to challenge the accuracy and pertinence of the cited documents. It will be clearly understood that, although a number of information sources, including scientific journal articles, patent documents, and textbooks, are referred to herein, this reference does not constitute an admission that any of these documents forms part of the common general knowledge in the art.

The discussion of the general methods given herein is intended for illustrative purposes only. Other alternative methods and embodiments will be apparent to those skilled in the art upon review of this disclosure. In addition, although various advantages, aspects, and objects of the present application have been discussed herein with reference to various embodiments, it will be understood that the scope of the application should not be limited by reference to such advantages, aspects, and objects. Rather, the scope of the application should be determined with reference to the appended claims.

### EXAMPLES

Additional alternatives are disclosed in further detail in the following examples, which are not in any way intended to limit the scope of the claims.

### Example 1

### Nucleic Acid Isolation

Total DNA from the collected biological samples was extracted using a standard DNA isolation protocol after a minimum of two days of storage at room temperature.

### Example 2

### DNA Quantification

Following DNA isolation, the human genomic DNA was resuspended in approximately 75 μL and an aliquot of this DNA was used for DNA quantification by using a validated PicoGreen® fluorescence assay protocol. The PicoGreen method uses fluorescence probes to detect the extracted human DNA, in which the amount of fluorescence was measured against a standardized concentration curve, followed by background noise correction, and then used to calculate the DNA concentration of each DNA specimen. Extracted DNA samples were either manually pipetted or automatically transferred to a Fluorotrac™ 200, 96-well plate for use on a BioTek™ Flx800™ Fluorescence Microplate Reader (Fluorometer).

### Example 3

### DNA Normalization and Integrity Evaluation

DNA samples were typically normalized to 50 ng/μl (L-0052) and, in most experiments, subsequently subjected to gel-based quality control (QC) analysis according to standard molecular biology methods. In some experiments, this normalization step was omitted. The plate of samples found to contain DNA concentrations of at least 20 ng/μL, as quantified by the PicoGreen method as described in Example 2, were subsequently normalized using the BioMek® FX Liquid Handler (Beckman Coulter). In particular, samples with DNA concentrations measured to be greater than 200 ng/μL were diluted 1:10 with UltraPure Distilled Water into the acceptable range. Samples with DNA concentrations measured to be between 50 ng/μL and 200 ng/μL were normalized to a concentration of 50 ng/μL in this step. Samples with DNA concentrations measured to be between 20 and 50 ng/μL were unchanged in this step. Additionally, the quality of the DNA in the samples was evaluated based on gel electrophoresis. The DNAs passed this gel-based quality control (QC) analysis, and meet DNA quantification criteria were subjected to further testing (samples containing high molecular weight genomic DNA was analyzed for integrity). DNA concentrations were typically >20 ng/μl. In some instances, however, DNA samples of 5 ng/μl or greater nucleic acid concentration were also admissible.

### Example 4

### Genotyping

Genotyping assays were designed for use with commercially synthesized nucleic acid primers (Integrated DNA Technologies, Coralville, Iowa). Samples were genotyped using Access Array or Juno technology (Fluidigm) for library preparation and/or enrichment. Next-generation sequencing (NGS) was performed using a MiSeq system or NextSeq system (Illumina, San Diego, Calif.), using targeted-sequencing preparation (TSP) chemistry, previously referred to as Orion chemistry. A typical workflow of the genotyping process includes the following steps:

1) Multiplex Step: this step typically involves multiplexed target enrichment and amplification to incorporate barcodes into the nucleic acids.

2) Harvest Step: this step involves harvesting pools of samples from the Access Array or Juno plates.

3) Post-Harvest Purification Step: this step involves purification of nucleic acids by using a Solid Phase Reversible Immobilization (SPRI) paramagnetic bead-based technique. This step can be repeated up to 3 times depending on samples quality and specific applications.

4) Adaptor Addition Step: after purification step, suitable adaptors were added to the purified nucleic acids by using an off-chip adaptor addition procedure to prepare for sequencing.

5) Cleanup Step: this step involves cleaning up the SPRI beads with DNA bound to the beads, which was typically performed one time and on-bead rather than in solution.

6) Sequencing: next generation sequencing (NGS) was performed by using a number of commercially available NGS methods and platforms. In most instances, next generation sequencing (NGS) was performed on a MiSeq platform or NextSeq platform (Illumina).

In a typical workflow, the steps discussed above were performed with Fluidigm Access Array™ or Juno™ systems that were specifically designed to support high-throughput re-sequencing, targeted enrichment, sample barcoding, and library preparation for sequencing using amplicon tagging. In most experiments, the 48.48 Access Array™ integrated fluidic circuits (IFCs) and the Juno™ LP 192.24 integrated fluidic circuits (IFCs) were used. The 48.48 Access Array™ IFC designed for next generation sequencing was a microfluidic chip that systematically combines 48 sample inputs with 48 primer inputs to create 2,304 combinations of samples and primers. Therefore, the 48.48 Access Array™ IFCs can process up to 48 samples, in up to 48 assay wells in parallel, where each well can be amplifying up to approximately 100 assays. The Juno™ LP 192.24 IFC designed for next generation sequencing was a microfluidic chip that systematically combines 192 sample inputs with 24 primer inputs to create 4,608 combinations of samples and primers. Therefore, the 192.24 Juno™ IFCs can process up to 192 samples, in up to 24 assay wells in parallel, where each well can be amplifying up to approximately 100 assays. Alternatively, in other experiments, IFCs with a different format were deployed, depending on specific samples and applications.

### Example 5

### Amplification Plate Set Up on 48.48 Access Array™ IFCs

A pooled assay mix was prepared by mixing the primers of the PCR-based assays designed to specifically scan the genomic region targeted by the PCR-based assays. Primers amplifying the different genetic targets, for instances, genetic variations or biomarkers, were multiplexed into one reaction of approximately 100 targets per reaction well. The amplification step allows for the enrichment of genomic sequences. The pooled assay mix was combined with a commercial Multiplex PCR Master Mix (Qiagen) to prepare an “Amp Master Mix”.

20× primer solutions were prepared by combining Orion Multiplex Primer Pool, TSP assay loading reagent, and water. The final volume per primer solution was 20 μl Orion Multiplex Primer Pool, 2.5 μl TSP assay loading reagent, and 27.5 μl water for a total of 50 μl.

Subsequently, a sample pre-mix was prepared such that each reaction contains 2.5 μl Qiagen Multiplex PCR mix, 0.25 μl TSP sample loading reagent, 0.2 μl Qiagen HotStar Taq DNA polymerase, and 0.05 μl water.

Separately, standard 96-well microtiter plates were prepared to hold 48 individual sample mix solutions. For the amplification step, set up with a liquid handler or manually, the final volume per reaction was 3.0 μL of Sample Pre Master, 1.0 μL of genomic DNA (gDNA, about 50 ng/μl), and 1.0 μl of barcode from the TSP barcode library. A list of exemplary of genes, biomarkers, and primers used in these experiments are presented in TABLE 3 for each patient specimen. Nucleotide positions of the amplicons relative to the respective chromosomal sequences are also provided in TABLE 3. Negative controls, No template controls (NTC), were also loaded, which provided an indication of whether any contamination occurred during sample processing. The microtiter plate was then sealed and vortexed to ensure proper reagent mixing, then briefly centrifuged to ensure reaction mix solutions assembled at the bottom of the wells.

The Access Array™ integrated fluidic circuit (IFC) was primed, followed by loading of the reaction reagents onto the chips. Four (4) μl of each 20× primer solution from the primer plate was loaded and four (4) μl of each sample mix from the samples plate were loaded into each of the sample inlets. Typically, the IFC was loaded into IFC controller, for instance, a pre-PCR IFC Controller AX (Fluidigm), and a predefined script was allowed to run in order to load the sample mix onto the chip.

### Example 6

### Amplification Plate Set Up on the Juno™ LP 192.24 IFCs

A pooled assay mix was prepared by mixing the primers of the PCR-based assays designed to specifically scan the genomic region targeted by the PCR-based assays. Primers amplifying the different genetic targets, for instances, genetic variations or biomarkers, were multiplexed into one reaction of approximately 100 targets per reaction well. The amplification step allows for the enrichment of genomic sequences. The pooled assay mix was combined with a commercial TSP Multiplex PCR Master Mix (Fluidigm) to prepare an “Amp Master Mix”.

10× primer solutions were prepared by combining Targeted DNA Sequence Library Multiplex Primer Pool, TSP assay loading reagent, and water. The final volume per primer solution was 10 μl Targeted DNA Sequence Library, 2.5 μl TSP assay loading reagent, and 37.5 μl water for a total of 50 μl.

Subsequently, a sample pre-mix was prepared such that each reaction contains 1.25 μl TSP 4× Multiplex PCR mix, 0.25 μl TSP Sample loading reagent, 0.2 μl TSP DNA polymerase, and 0.3 μl water.

Separately, two standard 96-well microtiter plates were prepared to hold 96 individual sample mix solutions. For the amplification step, set up with a liquid handler or manually, the final volume per reaction was 2.0 μL of Sample Pre Master, 2.0 μL of genomic DNA (gDNA, about 50 ng/μl), and 1.0 μl of barcode from the TSP barcode library. A list of exemplary of genes, biomarkers, and primers used in these experiments are presented in TABLE 3 for each patient specimen. Nucleotide positions of the amplicons relative to the respective chromosomal sequences are also provided in TABLE 3. Negative controls, No template controls (NTC), were also loaded, which provided an indication of whether any contamination occurred during sample processing. The microtiter plates were then sealed and vortexed to ensure proper reagent mixing, then briefly centrifuged to ensure reaction mix solutions assembled at the bottom of the wells.

The Juno™ integrated fluidic circuit (IFC) was prepared, followed by loading of the reaction reagents onto the chips. 3.5 μl of each 10× primer solution from the primer plate was loaded and 3.5 μl of each sample mix from the samples plate were loaded into each of the sample inlets. Typically, the IFC was sealed with the Barrier Type Applicator, was placed on the Juno™ system, and a predefined script was allowed to run in order to load the sample mix onto the chip. The Juno™ system combined IFC control, loading, thermal cycling and harvesting into one process step, after the IFC was finished; entire harvested volumes from the appropriate samples were combined into a single tube for next step (Library purification).

TABLE 3
Non-limiting examples of genes and biomarkers

| Gene/Biomarker | Amplicon Name | Chromosome | From | To |
|---|---|---|---|---|
| ACE | rs4646994_II_F1R1 | 17 | 61565740 | 61566076 |
| ACE | rs4646994_WT_F2R2 | 17 | 61565823 | 61565981 |
| ACE | rs4646994_WT_F3R3 | 17 | 61565831 | 61565961 |
| ACE | rs4646994_WT_F1R1 | 17 | 61565842 | 61566033 |
| CI083373 | CI083373_1549 | 1 | 152286006 | 152286230 |
| CI083373 | CI083373_t1_1184 | 1 | 152286048 | 152286245 |
| CM081617 | CM081617-r1-1_1925 | 1 | 152277346 | 152277534 |
| CM081617 | CM081617_1666 | 1 | 152277402 | 152277599 |
| FLG | FLG-r3-1_1455 | 1 | 152278577 | 152278798 |
| FLG | FLG-r2-1_1904 | 1 | 152278614 | 152278840 |
| MC1R | MC1R-r5-2_1644 | 16 | 89985953 | 89986173 |
| MC1R | MC1R-r5-2_1645 | 16 | 89986087 | 89986302 |
| MC1R | MC1R-r1-1_2796 | 16 | 89985874 | 89986112 |
| MC1R | MC1R-r2-1_1487 | 16 | 89986118 | 89986352 |
| MC1R | MC1R-r1-1_1189 | 16 | 89985708 | 89985939 |
| MC1R | MC1R-r6-1_1770 | 16 | 89985744 | 89985924 |
| MC1R | MC1R-r1-1_6940 | 16 | 89986362 | 89986601 |
| MC1R | MC1R-r1-1_7397 | 16 | 89986488 | 89986724 |
| MC1R | MC1R-r1-1_7232 | 16 | 89986525 | 89986758 |
| MC1R_CDS_01 | MC1R_CDS_01_F3R3a | 16 | 89986051 | 89986302 |
| MC1R_CDS_01 | MC1R_CDS_01_F3R3b | 16 | 89986053 | 89986352 |
| MC1R_CDS_01 | MC1R_CDS_01_8 | 16 | 89986039 | 89986263 |
| MC1R_CDS_01 | MC1R_CDS_01_F2R2a | 16 | 89985814 | 89986103 |
| MC1R_CDS_01 | MC1R_CDS_01_F2R2b | 16 | 89985817 | 89986116 |
| MC1R_CDS_01 | MC1R_CDS_01_5 | 16 | 89985853 | 89986081 |
| MC1R_CDS_01 | MC1R_CDS_01_3 | 16 | 89985708 | 89985938 |
| MC1R_CDS_01 | MC1R_CDS_01_F1R1a | 16 | 89985598 | 89985887 |
| MC1R_CDS_01 | MC1R_CDS_01_F1R1b | 16 | 89985610 | 89985893 |
| MC1R_CDS_01 | MC1R_CDS_01_17 | 16 | 89986431 | 89986624 |
| MC1R_CDS_01 | MC1R_CDS_01_F5R5b | 16 | 89986432 | 89986719 |
| MC1R_CDS_01 | MC1R_CDS_01_F5R5a | 16 | 89986485 | 89986777 |
| rs1001179 | rs1001179_t1_1707 | 11 | 34460104 | 34460325 |
| rs1001179 | rs1001179_1366 | 11 | 34460122 | 34460342 |
| rs1015362 | rs1015362-r1-1_1272 | 20 | 32738480 | 32738714 |
| rs1015362 | rs1015362_1438 | 20 | 32738497 | 32738733 |
| rs1042602 | rs1042602-r2-1_1834 | 11 | 88911545 | 88911760 |
| rs1042602 | rs1042602_1699 | 11 | 88911581 | 88911794 |
| rs1049346 | rs1049346_1162 | 6 | 38670722 | 38670931 |
| rs1049346 | rs1049346_2 | 6 | 38670735 | 38670895 |
| rs1049346 | rs1049346_t1_148 | 6 | 38670739 | 38670962 |
| rs1050450 | rs1050450-r2-1_1866 | 3 | 49394678 | 49394888 |
| rs1050450 | rs1050450_1338 | 3 | 49394731 | 49394946 |
| rs10798036 | rs10798036_F1R1 | 1 | 186052815 | 186053057 |
| rs10798036 | rs10798036_1442 | 1 | 186052875 | 186053056 |
| rs10798036 | rs10798036-r1-1_566 | 1 | 186052896 | 186053076 |
| rs1110400 | rs1110400_2 | 16 | 89986086 | 89986250 |
| rs1110400 | rs1110400_3 | 16 | 89986082 | 89986263 |
| rs111314066 | rs111314066_1 | 5 | 144469808 | 144470007 |
| rs111314066 | rs111314066_2 | 5 | 144469824 | 144469983 |
| rs111314066 | rs111314066_F1R1 | 5 | 144469727 | 144469971 |
| rs1126809 | rs1126809_1 | 11 | 89017830 | 89018010 |
| rs1126809 | rs1126809-r1-1_1631 | 11 | 89017907 | 89018086 |
| rs1126809 | rs1126809_F1R1 | 11 | 89017909 | 89018170 |
| rs1130534 | rs1130534_F1R1 | 6 | 38650364 | 38650650 |
| rs1130534 | rs1130534-r2-1_1736 | 6 | 38650539 | 38650723 |
| rs1130534 | rs1130534-r2-1_7 | 6 | 38650556 | 38650737 |
| rs11547464 | rs11547464_rs1805007_F1R1 | 16 | 89986046 | 89986267 |
| rs11547464 | rs11547464_2 | 16 | 89986039 | 89986200 |
| rs11547464 | rs11547464-r2-1_3 | 16 | 89985953 | 89986135 |
| rs11549465 | rs11549465_1422 | 14 | 62207431 | 62207658 |
| rs11549465 | rs11549465_t1_1972 | 14 | 62207472 | 62207683 |
| rs121909626 | rs121909626_F2R2 | 1 | 152279546 | 152279827 |
| rs121909626 | rs121909626_1817 | 1 | 152279588 | 152279821 |
| rs121909626 | rs121909626_F1R1 | 1 | 152279663 | 152279832 |
| rs12191877 | rs12191877_1593 | 6 | 31252799 | 31252992 |
| rs12191877 | rs12191877-r2-1_309 | 6 | 31252851 | 31253080 |
| rs12203592 | rs12203592_1305 | 6 | 396205 | 396432 |
| rs12203592 | rs12203592_t1_1408 | 6 | 396226 | 396456 |
| rs12210050 | rs12210050_1985 | 6 | 475364 | 475560 |
| rs12210050 | rs12210050-r1-1_148 | 6 | 475383 | 475596 |
| rs12272004 | rs12272004_t1_190 | 11 | 116603594 | 116603816 |
| rs12272004 | rs12272004_1492 | 11 | 116603622 | 116603835 |
| rs1265181 | rs1265181_F1R1 | 6 | 31155615 | 31155842 |
| rs1265181 | rs1265181_F1R1b | 6 | 31155615 | 31155827 |
| rs1265181 | rs1265181_F2R2 | 6 | 31155740 | 31155943 |
| rs12913832 | rs12913832-r1-1_181 | 15 | 28365493 | 28365726 |
| rs12913832 | rs12913832-r2-1_754 | 15 | 28365533 | 28365770 |
| rs12934922 | rs12934922_t1_1228 | 16 | 81301580 | 81301791 |
| rs12934922 | rs12934922_1546 | 16 | 81301606 | 81301817 |
| rs138726443 | rs138726443_1592 | 1 | 152279943 | 152280136 |
| rs1393350 | rs1393350_1389 | 11 | 89010928 | 89011161 |
| rs1393350 | rs1393350_t1_1176 | 11 | 89010970 | 89011181 |
| rs1426654 | rs1426654_1665 | 15 | 48426367 | 48426579 |
| rs1426654 | rs1426654-r2-1_1494 | 15 | 48426403 | 48426633 |
| rs150597413 | rs150597413_1334 | 1 | 152277511 | 152277742 |
| rs150597413 | rs150597413_t1_175 | 1 | 152277527 | 152277757 |
| rs1540771 | rs1540771_1247 | 6 | 465913 | 466152 |
| rs1540771 | rs1540771_t1_192 | 6 | 465946 | 466132 |
| rs16891982 | rs16891982-r2-1_299 | 5 | 33951553 | 33951766 |
| rs16891982 | rs16891982_1666 | 5 | 33951599 | 33951815 |
| rs174547 | rs174547_t1_1524 | 11 | 61570647 | 61570879 |
| rs174547 | rs174547_1119 | 11 | 61570681 | 61570907 |
| rs17553719 | rs17553719_1297 | 9 | 33447450 | 33447639 |
| rs17553719 | rs17553719-r2-1_3 | 9 | 33447459 | 33447625 |
| rs17553719 | rs17553719_1110 | 9 | 33447471 | 33447680 |
| rs17728338 | rs17728338-r1-1_606 | 5 | 150478182 | 150478403 |
| rs17728338 | rs17728338_1649 | 5 | 150478241 | 150478420 |
| rs1799750 | rs1799750_3 | 11 | 102670354 | 102670537 |
| rs1799750 | rs1799750_1779 | 11 | 102670378 | 102670591 |
| rs1799750 | rs1799750_1110 | 11 | 102670386 | 102670614 |
| rs1799752 | rs1799752-r2-1_1891 | 17 | 61565739 | 61565959 |
| rs1799752 | rs1799752_1474 | 17 | 61565766 | 61565976 |
| rs1800566 | rs1800566_1874 | 16 | 69745018 | 69745231 |
| rs1800566 | rs1800566_1408 | 16 | 69745062 | 69745275 |
| rs1800624 | rs1800624_1137 | 6 | 32152262 | 32152491 |
| rs1800625 | rs1800625-r1-1_1386 | 6 | 32152318 | 32152529 |
| rs1801131 | rs1801131_t1_124 | 1 | 11854358 | 11854574 |
| rs1801131 | rs1801131_1179 | 1 | 11854381 | 11854592 |
| rs1801133 | rs1801133_194 | 1 | 11856258 | 11856485 |
| rs1801133 | rs1801133_t1_1238 | 1 | 11856294 | 11856513 |
| rs1805005 | rs1805005_2 | 16 | 89985772 | 89985939 |
| rs1805005 | rs1805005_1 | 16 | 89985714 | 89985902 |
| rs1805006 | rs1805006-r2-1_1161 | 16 | 89985781 | 89985970 |
| rs1805006 | rs1805006_2 | 16 | 89985872 | 89986061 |
| rs1805007 | rs1805007_1322 | 16 | 89986003 | 89986220 |
| rs1805007 | rs11547464_rs1805007_F2R2 | 16 | 89986051 | 89986220 |
| rs1805008 | rs1805008_3 | 16 | 89986084 | 89986267 |
| rs1805009 | rs1805009_1955 | 16 | 89986434 | 89986654 |
| rs1805009 | rs1805009_2 | 16 | 89986487 | 89986676 |
| rs1805009 | rs1805009_1 | 16 | 89986434 | 89986602 |
| rs200519781 | rs200519781-r2-1_11 | 1 | 152283893 | 152284103 |
| rs200519781 | rs200519781_16 | 1 | 152283921 | 152284140 |
| rs20541 | rs20541_1514 | 5 | 131995852 | 131996070 |
| rs20541 | rs20541_t1_1386 | 5 | 131995883 | 131996099 |
| rs2070600 | rs2070600_t1_1192 | 6 | 32151309 | 32151532 |
| rs2070600 | rs2070600_111 | 6 | 32151342 | 32151553 |
| rs2082412 | rs2082412_t1_1897 | 5 | 158717660 | 158717883 |
| rs2082412 | rs2082412_1242 | 5 | 158717681 | 158717906 |
| rs2201841 | rs2201841_1108 | 1 | 67694110 | 67694326 |
| rs2201841 | rs2201841_F1R1 | 1 | 67694082 | 67694339 |
| rs2201841 | rs2201841_F2R2 | 1 | 67694105 | 67694333 |
| rs2228479 | rs2228479-r2-1_1118 | 16 | 89985853 | 89986076 |
| rs2282679 | rs2282679-r2-1_1594 | 4 | 72608231 | 72608415 |
| rs2282679 | rs2282679_M_REP | 4 | 72608239 | 72608424 |
| rs2282679 | rs2282679_F1R1 | 4 | 72608248 | 72608477 |
| rs2555364 | rs2555364_1652 | 15 | 48419283 | 48419496 |
| rs2555364 | rs2555364_t1_1994 | 15 | 48419301 | 48419521 |
| rs26722 | rs26722_t1_1498 | 5 | 33963736 | 33963959 |
| rs26722 | rs26722_1351 | 5 | 33963774 | 33963986 |
| rs2917666 | rs2917666_1439 | 16 | 69763835 | 69764015 |
| rs2917666 | rs2917666_1378 | 16 | 69763839 | 69764033 |
| rs322458 | rs322458-r1-1_1671 | 3 | 120585195 | 120585407 |
| rs322458 | rs322458-r1-1_1791 | 3 | 120585213 | 120585432 |
| rs33972313 | rs33972313_t1_1989 | 5 | 138715380 | 138715563 |
| rs33972313 | rs33972313_1103 | 5 | 138715401 | 138715580 |
| rs35318931 | rs35318931_151 | X | 38008995 | 38009214 |
| rs35318931 | rs35318931_t1_127 | X | 38009017 | 38009197 |
| rs374588791 | rs374588791_1317 | 1 | 152279972 | 152280192 |
| rs397507563 | rs397507563_1173 | 1 | 152283526 | 152283717 |
| rs397507563 | rs397507563_1249 | 1 | 152283589 | 152283768 |
| rs429358 | rs429358_t1_1648 | 19 | 45411805 | 45412038 |
| rs429358 | rs429358_143 | 19 | 45411828 | 45412066 |
| rs429358 | rs429358_2 | 19 | 45411879 | 45412068 |
| rs429358 | rs429358_1 | 19 | 45411828 | 45412037 |
| rs4340 | rs4340_12 | 17 | 61565743 | 61565953 |
| rs4340 | rs4340_6 | 17 | 61565865 | 61566030 |
| rs4654748 | rs4654748_1406 | 1 | 21785956 | 21786153 |
| rs4654748 | rs4654748_t1_1182 | 1 | 21785990 | 21786203 |
| rs4746 | rs4746_F1R1 | 6 | 38650437 | 38650736 |
| rs4746 | rs4746-r2-1_3 | 6 | 38650553 | 38650771 |
| rs4746 | rs4746_1 | 6 | 38650565 | 38650754 |
| rs4880 | rs4880_3 | 6 | 160113724 | 160113917 |
| rs4880 | rs4880_1271 | 6 | 160113754 | 160113975 |
| rs4880 | rs4880_t1_1131 | 6 | 160113778 | 160113958 |
| rs4911414 | rs4911414_t1_1235 | 20 | 32729312 | 32729539 |
| rs4911414 | rs4911414_1811 | 20 | 32729351 | 32729560 |
| rs4911442 | rs4911442_t1_1476 | 20 | 33354892 | 33355077 |
| rs4911442 | rs4911442-r2-1_1334 | 20 | 33354915 | 33355099 |
| rs558269137 | rs558269137-r2-1_48 | 1 | 152284926 | 152285162 |
| rs558269137 | rs558269137-r1-1_97 | 1 | 152284956 | 152285180 |
| rs602662 | rs602662_1829 | 19 | 49206867 | 49207076 |
| rs602662 | rs602662_t1_1548 | 19 | 49206908 | 49207119 |
| rs610604 | rs610604_t1_1903 | 6 | 138199284 | 138199487 |
| rs610604 | rs610604_1843 | 6 | 138199333 | 138199542 |
| rs61816761 | rs61816761_1345 | 1 | 152285736 | 152285970 |
| rs61816761 | rs61816761_t1_1692 | 1 | 152285780 | 152285991 |
| rs7412 | rs7412_2 | 19 | 45411923 | 45412104 |
| rs7412 | rs7412_t1_1728 | 19 | 45411966 | 45412176 |
| rs7412 | rs7412_F2R2 | 19 | 45411987 | 45412231 |
| rs7412 | rs7412_1430 | 19 | 45411987 | 45412203 |
| rs7501331 | rs7501331-r2-1_1849 | 16 | 81314354 | 81314591 |
| rs7501331 | rs7501331_1594 | 16 | 81314405 | 81314618 |
| rs7594220 | rs7594220_t1_1877 | 2 | 643223 | 643454 |
| rs7594220 | rs7594220_1230 | 2 | 643247 | 643426 |
| rs763035 | rs763035-r3-1_4 | 6 | 32394693 | 32394923 |
| rs763035 | rs763035_1 | 6 | 32394749 | 32394940 |
| rs7787362 | rs7787362_1557 | 7 | 73392486 | 73392703 |
| rs7787362 | rs7787362_t1_1826 | 7 | 73392551 | 73392735 |
| rs885479 | rs885479_1966 | 16 | 89986037 | 89986262 |
| rs885479 | rs885479_2 | 16 | 89986089 | 89986278 |
| rs885479 | rs885479_3 | 16 | 89986004 | 89986220 |

### Example 7

### Amplification

After retrieving from the pre-PCR IFC controller, the Access Array™ IFC was placed into a Fluidigm FC1™ cycler and an Access Array™ Orion protocol was run on the Fluidigm FC™ cycler.

Samples were then amplified. A typical amplification protocol includes (1) 18 cycles of 15 seconds at 95° C., (2) 90 seconds at 60° C., and (3) 90 seconds at 68° C., which was followed by a final extension for 3 minutes at 68° C. The amplified products were then harvested on a post-PCR ICF Controller AX. After harvest, amplified products were cleaned up by using a SPRI paramagnetic bead-based procedure, which can be repeated up to 3 times, and quantitated with a Qubit™ system (Invitrogen) or a Tapestation system (Agilent) or equivalent.

The library comprising the amplified products was then prepared for an adaptor-addition PCR step. For this purpose, Qiagen Multiplex PCR master mix was prepared for use with the TSP adaptor Mix and the purified library, where the final composition per reaction was 15 μl master mix, 6 μl TSP adaptor mix and 4.5 μl water for a total of 30 μl final volume. Samples were subsequently amplified via PCR. A typical amplification protocol includes (1) 10 cycles of 15 seconds at 95° C., (2) 90 seconds at 60° C., and (3) 90 seconds at 68° C., which was followed by a final extension for 3 minutes at 68° C.

After amplification, the library generated from both the Access Array™ system and the Juno™ system were subject to a clean-up step by using a SPRI paramagnetic bead-based procedure, and quantitated on a Qubit™ system (Invitrogen) or a Tapestation system (Agilent) or equivalent. Subsequently, the final library was subject to the high-throughput sequencing step by using a MiSeq system or NextSeq system (Illumina, San Diego, Calif.). In some instances, the primers were designed such that the nucleotide sequences span multiple biomarkers located adjacent to one another in the genome (TABLE 4).

TABLE 4
Non-limiting examples of amplicons whose nucleotide
sequences span multiple adjacent biomarkers

| Amplicon ID | Biomarker ID |
|---|---|
| CI083373_1549 | 1249insG |
| CI083373_t1_1184 | 1249insG |
| CM081617_1666 | rs761212672 |
| CM081617-r1-1_1925 | rs761212672 |
| FLG-r2-1_1904 | rs540453626 |
| FLG-r2-1_1904 | rs578153418 |
| FLG-r3-1_1455 | rs540453626 |
| FLG-r3-1_1455 | rs578153418 |
| MC1R_CDS_01_17 | rs1805009 |
| MC1R_CDS_01_3 | rs1805005 |
| MC1R_CDS_01_3 | rs1805006 |
| MC1R_CDS_01_5 | rs1805006 |
| MC1R_CDS_01_5 | rs2228479 |
| MC1R_CDS_01_8 | rs1110400 |
| MC1R_CDS_01_8 | rs11547464 |
| MC1R_CDS_01_8 | rs1805007 |
| MC1R_CDS_01_8 | rs1805008 |
| MC1R_CDS_01_8 | rs885479 |
| MC1R_CDS_01_F1R1a | rs1805005 |
| MC1R_CDS_01_F1R1b | rs1805005 |
| MC1R_CDS_01_F2R2a | rs11547464 |
| MC1R_CDS_01_F2R2a | rs1805005 |
| MC1R_CDS_01_F2R2a | rs1805006 |
| MC1R_CDS_01_F2R2a | rs2228479 |
| MC1R_CDS_01_F2R2b | rs11547464 |
| MC1R_CDS_01_F2R2b | rs1805005 |
| MC1R_CDS_01_F2R2b | rs1805006 |
| MC1R_CDS_01_F2R2b | rs2228479 |
| MC1R_CDS_01_F3R3a | rs1110400 |
| MC1R_CDS_01_F3R3a | rs11547464 |
| MC1R_CDS_01_F3R3a | rs1805007 |
| MC1R_CDS_01_F3R3a | rs1805008 |
| MC1R_CDS_01_F3R3a | rs885479 |
| MC1R_CDS_01_F3R3b | rs1110400 |
| MC1R_CDS_01_F3R3b | rs11547464 |
| MC1R_CDS_01_F3R3b | rs1805007 |
| MC1R_CDS_01_F3R3b | rs1805008 |
| MC1R_CDS_01_F3R3b | rs885479 |
| MC1R_CDS_01_F5R5a | rs1805009 |
| MC1R_CDS_01_F5R5b | rs1805009 |
| MC1R-r1-1_1189 | rs1805005 |
| MC1R-r1-1_1189 | rs1805006 |
| MC1R-r1-1_2796 | rs11547464 |
| MC1R-r1-1_2796 | rs1805006 |
| MC1R-r1-1_2796 | rs2228479 |
| MC1R-r1-1_6940 | rs1805009 |
| MC1R-r1-1_7232 | rs1805009 |
| MC1R-r1-1_7397 | rs1805009 |
| MC1R-r2-1_1487 | rs1110400 |
| MC1R-r2-1_1487 | rs1805008 |
| MC1R-r2-1_1487 | rs885479 |
| MC1R-r5-2_1644 | rs1110400 |
| MC1R-r5-2_1644 | rs11547464 |
| MC1R-r5-2_1644 | rs1805007 |
| MC1R-r5-2_1644 | rs1805008 |
| MC1R-r5-2_1644 | rs885479 |
| MC1R-r5-2_1645 | rs1110400 |
| MC1R-r5-2_1645 | rs11547464 |
| MC1R-r5-2_1645 | rs1805007 |
| MC1R-r5-2_1645 | rs1805008 |
| MC1R-r5-2_1645 | rs885479 |
| MC1R-r6-1_1770 | rs1805005 |
| MC1R-r6-1_1770 | rs1805006 |
| rs1001179_1366 | rs1001179 |
| rs1001179_t1_1707 | rs1001179 |
| rs1015362_1438 | rs1015362 |
| rs1015362-r1-1_1272 | rs1015362 |
| rs1042602_1699 | rs1042602 |
| rs1042602-r2-1_1834 | rs1042602 |
| rs1049346_1162 | rs1049346 |
| rs1049346_2 | rs1049346 |
| rs1049346_t1_148 | rs1049346 |
| rs1050450_1338 | rs1050450 |
| rs1050450-r2-1_1866 | rs1050450 |
| rs10798036_1442 | rs10798036 |
| rs10798036_F1R1 | rs10798036 |
| rs10798036-r1-1_566 | rs10798036 |
| rs1110400_2 | rs1110400 |
| rs1110400_2 | rs11547464 |
| rs1110400_2 | rs1805007 |
| rs1110400_2 | rs1805008 |
| rs1110400_2 | rs885479 |
| rs1110400_3 | rs1110400 |
| rs1110400_3 | rs11547464 |
| rs1110400_3 | rs1805007 |
| rs1110400_3 | rs1805008 |
| rs1110400_3 | rs885479 |
| rs111314066_1 | rs111314066 |
| rs111314066_2 | rs111314066 |
| rs111314066_F1R1 | rs111314066 |
| rs1126809_1 | rs1126809 |
| rs1126809_F1R1 | rs1126809 |
| rs1126809-r1-1_1631 | rs1126809 |
| rs1130534_F1R1 | rs1130534 |
| rs1130534-r2-1_1736 | rs1130534 |
| rs1130534-r2-1_7 | rs1130534 |
| rs11547464_2 | rs1110400 |
| rs11547464_2 | rs11547464 |
| rs11547464_2 | rs1805007 |
| rs11547464_2 | rs1805008 |
| rs11547464_2 | rs885479 |
| rs11547464_rs1805007_F1R1 | rs1110400 |
| rs11547464_rs1805007_F1R1 | rs11547464 |
| rs11547464_rs1805007_F1R1 | rs1805007 |
| rs11547464_rs1805007_F1R1 | rs1805008 |
| rs11547464_rs1805007_F1R1 | rs885479 |
| rs11547464_rs1805007_F2R2 | rs1110400 |
| rs11547464_rs1805007_F2R2 | rs11547464 |
| rs11547464_rs1805007_F2R2 | rs1805007 |
| rs11547464_rs1805007_F2R2 | rs1805008 |
| rs11547464_rs1805007_F2R2 | rs885479 |
| rs11547464-r2-1_3 | rs1110400 |
| rs11547464-r2-1_3 | rs11547464 |
| rs11547464-r2-1_3 | rs1805007 |
| rs11549465_1422 | rs11549465 |
| rs11549465_t1_1972 | rs11549465 |
| rs121909626_1817 | rs121909626 |
| rs121909626_F1R1 | rs121909626 |
| rs121909626_F2R2 | rs121909626 |
| rs12191877_1593 | rs12191877 |
| rs12191877-r2-1_309 | rs12191877 |
| rs12203592_1305 | rs12203592 |
| rs12203592_t1_1408 | rs12203592 |
| rs12210050_1985 | rs12210050 |
| rs12210050-r1-1_148 | rs12210050 |
| rs12272004_1492 | rs12272004 |
| rs12272004_t1_190 | rs12272004 |
| rs1265181_F1R1 | rs1265181 |
| rs1265181_F1R1b | rs1265181 |
| rs1265181_F2R2 | rs1265181 |
| rs12913832-r1-1_181 | rs12913832 |
| rs12913832-r2-1_754 | rs12913832 |
| rs12934922_1546 | rs12934922 |
| rs12934922_t1_1228 | rs12934922 |
| rs138726443_1592 | rs138726443 |
| rs138726443_1592 | rs374588791 |
| rs1393350_1389 | rs1393350 |
| rs1393350_t1_1176 | rs1393350 |
| rs1426654_1665 | rs1426654 |
| rs1426654-r2-1_1494 | rs1426654 |
| rs150597413_1334 | rs150597413 |
| rs150597413_t1_175 | rs150597413 |
| rs1540771_1247 | rs1540771 |
| rs1540771_t1_192 | rs1540771 |
| rs16891982_1666 | rs16891982 |
| rs16891982-r2-1_299 | rs16891982 |
| rs174547_1119 | rs174547 |
| rs174547_t1_1524 | rs174547 |
| rs17553719_1110 | rs17553719 |
| rs17553719_1297 | rs17553719 |
| rs17553719-r2-1_3 | rs17553719 |
| rs17728338_1649 | rs17728338 |
| rs17728338-r1-1_606 | rs17728338 |
| rs1799750_1110 | rs1799750 |
| rs1799750_1779 | rs1799750 |
| rs1799750_3 | rs1799750 |
| rs1799752_1474 | rs1799752 |
| rs1799752-r2-1_1891 | rs1799752 |
| rs1800566_1408 | rs1800566 |
| rs1800566_1874 | rs1800566 |
| rs1800624_1137 | rs1800624 |
| rs1800624_1137 | rs1800625 |
| rs1800625-r1-1_1386 | rs1800624 |
| rs1800625-r1-1_1386 | rs1800625 |
| rs1801131_1179 | rs1801131 |
| rs1801131_t1_124 | rs1801131 |
| rs1801133_194 | rs1801133 |
| rs1801133_t1_1238 | rs1801133 |
| rs1805005_1 | rs1805005 |
| rs1805005_2 | rs1805005 |
| rs1805005_2 | rs1805006 |
| rs1805006_2 | rs1805006 |
| rs1805006_2 | rs2228479 |
| rs1805006-r2-1_1161 | rs1805005 |
| rs1805006-r2-1_1161 | rs1805006 |
| rs1805006-r2-1_1161 | rs2228479 |
| rs1805007_1322 | rs1110400 |
| rs1805007_1322 | rs11547464 |
| rs1805007_1322 | rs1805007 |
| rs1805007_1322 | rs1805008 |
| rs1805007_1322 | rs885479 |
| rs1805008_3 | rs1110400 |
| rs1805008_3 | rs11547464 |
| rs1805008_3 | rs1805007 |
| rs1805008_3 | rs1805008 |
| rs1805008_3 | rs885479 |
| rs1805009_1 | rs1805009 |
| rs1805009_1955 | rs1805009 |
| rs1805009_2 | rs1805009 |
| rs200519781_16 | rs200519781 |
| rs200519781-r2-1_11 | rs200519781 |
| rs20541_1514 | rs20541 |
| rs20541_t1_1386 | rs20541 |
| rs2070600_111 | rs2070600 |
| rs2070600_t1_1192 | rs2070600 |
| rs2082412_1242 | rs2082412 |
| rs2082412_t1_1897 | rs2082412 |
| rs2201841_1108 | rs2201841 |
| rs2201841_F1R1 | rs2201841 |
| rs2201841_F2R2 | rs2201841 |
| rs2228479-r2-1_1118 | rs1805006 |
| rs2228479-r2-1_1118 | rs2228479 |
| rs2282679_F1R1 | rs2282679 |
| rs2282679_M_REP | rs2282679 |
| rs2282679-r2-1_1594 | rs2282679 |
| rs2555364_1652 | rs2555364 |
| rs2555364_t1_1994 | rs2555364 |
| rs26722_1351 | rs26722 |
| rs26722_t1_1498 | rs26722 |
| rs2917666_1378 | rs2917666 |
| rs2917666_1439 | rs2917666 |
| rs322458-r1-1_1671 | rs322458 |
| rs322458-r1-1_1791 | rs322458 |
| rs33972313_1103 | rs33972313 |
| rs33972313_t1_1989 | rs33972313 |
| rs35318931_151 | rs35318931 |
| rs35318931_t1_127 | rs35318931 |
| rs374588791_1317 | rs138726443 |
| rs374588791_1317 | rs374588791 |
| rs397507563_1173 | rs397507563 |
| rs397507563_1249 | rs397507563 |
| rs429358_1 | rs429358 |
| rs429358_143 | rs429358 |
| rs429358_2 | rs429358 |
| rs429358_t1_1648 | rs429358 |
| rs4340_12 | rs1799752 |
| rs4340_6 | rs1799752 |
| rs4646994_II_F1R1 | rs1799752 |
| rs4646994_WT_F1R1 | rs1799752 |
| rs4646994_WT_F2R2 | rs1799752 |
| rs4646994_WT_F3R3 | rs1799752 |
| rs4654748_1406 | rs4654748 |
| rs4654748_t1_1182 | rs4654748 |
| rs4746_1 | rs1130534 |
| rs4746_F1R1 | rs1130534 |
| rs4746-r2-1_3 | rs1130534 |
| rs4880_1271 | rs4880 |
| rs4880_3 | rs4880 |
| rs4880_t1_1131 | rs4880 |
| rs4911414_1811 | rs4911414 |
| rs4911414_t1_1235 | rs4911414 |
| rs4911442_t1_1476 | rs4911442 |
| rs4911442-r2-1_1334 | rs4911442 |
| rs558269137-r1-1_97 | rs558269137 |
| rs558269137-r2-1_48 | rs558269137 |
| rs602662_1829 | rs602662 |
| rs602662_t1_1548 | rs602662 |
| rs610604_1843 | rs610604 |
| rs610604_t1_1903 | rs610604 |
| rs61816761_1345 | rs61816761 |
| rs61816761_t1_1692 | rs61816761 |
| rs7412_1430 | rs7412 |
| rs7412_2 | rs429358 |
| rs7412_2 | rs7412 |
| rs7412_F2R2 | rs7412 |
| rs7412_t1_1728 | rs7412 |
| rs7501331_1594 | rs7501331 |
| rs7501331-r2-1_1849 | rs7501331 |
| rs7594220_1230 | rs7594220 |
| rs7594220_t1_1877 | rs7594220 |
| rs763035_1 | rs763035 |
| rs763035-r3-1_4 | rs763035 |
| rs7787362_1557 | rs7787362 |
| rs7787362_t1_1826 | rs7787362 |
| rs885479_1966 | rs1110400 |
| rs885479_1966 | rs11547464 |
| rs885479_1966 | rs1805007 |
| rs885479_1966 | rs1805008 |
| rs885479_1966 | rs885479 |
| rs885479_2 | rs1110400 |
| rs885479_2 | rs11547464 |
| rs885479_2 | rs1805007 |
| rs885479_2 | rs1805008 |
| rs885479_2 | rs885479 |
| rs885479_3 | rs1110400 |
| rs885479_3 | rs11547464 |
| rs885479_3 | rs1805007 |
| rs885479_3 | rs1805008 |
| rs885479_3 | rs885479 |

Results: Genotyping results were analyzed using an internal pipeline for NGS analysis for genotyping by using proprietary algorithm or system of algorithms, wherein the likelihood of a patient exhibiting one or more phenotypic attributes based on the individual's genotype was assigned to categorical grades such as one of the following categories: High Risk, Very High Risk, Increased Risk, Diminished/Decreased Risk, or Typical/Normal/Average Risk. A preferred assessment table is provided below in TABLE 5.

TABLE 5
Skin Health Characteristics and Associated Biomarkers

| | | |
|---|---|---|
| Phenotype and Gene | Assigned Grade | SNP |

Skin Photo-Aging
Tanning Response

| | | |
|---|---|---|
| EXOC2 | Decreased Risk | rs12210050 |
| | Normal | rs12210050 |
| IRF4 | Decreased Risk | rs12203592 |
| | Normal | rs12203592 |
| HERC2 | Decreased Risk | rs12913832 |
| | Normal | rs12913832 |
| TYR | Decreased Risk | rs1393350 |
| | Normal | rs1393350 |
| TYR | Decreased Risk | rs1126809 |
| | Normal | rs1126809 |
| TYR | Decreased Risk | rs1042602 |
| | Normal | rs1042602 |
| SLC45A2 (MATP) | Decreased Risk | rs16891982 |
| | Normal | rs16891982 |
| SLC45A2 (MATP) | Decreased Risk | rs26722 |
| | Normal | rs26722 |
| SLC24A5 | Decreased Risk | rs1426654 |
| | Normal | rs1426654 |
| SLC24A5 | Decreased Risk | rs2555364 |
| | Normal | rs2555364 |
| ASIP Region | Decreased Risk | rs1015362 |
| | Normal | rs1015362 |
| ASIP Region | Decreased Risk | rs4911414 |
| | Normal | rs4911414 |
| NCOA6 | Decreased Risk | rs4911442 |
| | Normal | rs4911442 |

Sun Spots (Lentigines)

| | | |
|---|---|---|
| MC1R | Increased Risk | rs1805005 |
| | Typical Risk | rs1805005 |
| MC1R | Increased Risk | rs2228479 |
| | Typical Risk | rs2228479 |
| MC1R | Increased Risk | rs885479 |
| | Typical Risk | rs885479 |
| MC1R | Increased Risk | rs1805007 |
| | Typical Risk | rs1805007 |
| MC1R | Increased Risk | rs1805008 |
| | Typical Risk | rs1805008 |
| MC1R | Increased Risk | rs1805009 |
| | Typical Risk | rs1805009 |
| MC1R | Increased Risk | rs11547464 |
| | Typical Risk | rs11547464 |
| MC1R | Increased Risk | rs1110400 |
| | Typical Risk | rs1110400 |
| MC1R | Increased Risk | rs1805006 |
| | Typical Risk | rs1805006 |

Freckles (Ephelides)

| | | |
|---|---|---|
| IRF4 | Very High Risk | rs12203592 |
| | High Risk | rs12203592 |
| | Typical Risk | rs12203592 |
| MC1R | Very High Risk | rs1805007 |
| | High Risk | rs1805007 |
| | Typical Risk | rs1805007 |
| MC1R | Very High Risk | rs1805008 |
| | High Risk | rs1805008 |
| | Typical Risk | rs1805008 |
| MC1R | Very High Risk | rs1805009 |
| | High Risk | rs1805009 |
| | Typical Risk | rs1805009 |
| MC1R | Very High Risk | rs11547464 |
| | High Risk | rs11547464 |
| | Typical Risk | rs11547464 |
| 6p25.3-Intergenic between | Very High Risk | rs1540771 |
| EXOC2 and IRF4 | High Risk | rs1540771 |
| | Typical Risk | rs1540771 |
| NCOA6 | Very High Risk | rs4911442 |
| | High Risk | rs4911442 |
| | Typical Risk | rs4911442 |
| ASIP Region | Very High Risk | rs4911414 |
| | High Risk | rs4911414 |
| | Typical Risk | rs4911414 |
| TYR | Very High Risk | rs1042602 |
| | High Risk | rs1042602 |
| | Typical Risk | rs1042602 |
| TYR | Very High Risk | rs1393350 |
| | High Risk | rs1393350 |
| | Typical Risk | rs1393350 |

Wrinkles and Collagen Degradation

| | | |
|---|---|---|
| STXBP5L | Increased Risk | rs322458 |
| | Typical Risk | rs322458 |
| MMP1 | Increased Risk | rs1799750 |
| | Typical Risk | rs1799750 |

Skin Texture and Elasticity
Cellulite

| | | |
|---|---|---|
| ACE | Increased Risk | rs1799752 |
| | Typical Risk | rs1799752 |
| | Reduced Risk | rs1799752 |
| HIF1A | Increased Risk | rs11549465 |
| | Typical Risk | rs11549465 |
| | Reduced Risk | rs11549465 |

Stretch Marks (Striae Distensae)

| | | |
|---|---|---|
| ELN | Increased Risk | rs7787362 |
| | Typical Risk | rs7787362 |
| SRPX | Increased Risk | rs35318931 |
| | Typical Risk | rs35318931 |
| HMCN1 | Increased Risk | rs10798036 |
| | Typical Risk | rs10798036 |
| TMEM18 | Increased Risk | rs7594220 |
| | Typical Risk | rs7594220 |

Varicose Veins

| | | |
|---|---|---|
| MTHFR | Increased Risk | rs1801133 |
| | Typical Risk | rs1801133 |
| MTHFR | Increased Risk | rs1801131 |
| | Typical Risk | rs1801131 |

Skin Moisture Factor
Dry Skin (Ichthyosis)

| | | |
|---|---|---|
| FLG | Increased Risk | rs558269137 |
| | Typical Risk | rs558269137 |
| FLG | Increased Risk | rs61816761 |
| | Typical Risk | rs61816761 |
| FLG | Increased Risk | rs138726443 |
| | Typical Risk | rs138726443 |
| FLG | Increased Risk | rs150597413 |
| | Typical Risk | rs150597413 |
| FLG | Increased Risk | rs397507563 |
| | Typical Risk | rs397507563 |
| FLG | Increased Risk | rs200519781 |
| | Typical Risk | rs200519781 |

Hydration Capacity

| | | |
|---|---|---|
| AQP3 | Decreased Risk | rs17553719 |
| | Normal | rs17553719 |

Skin Inflammation and Allergy Risk
Eczema (Atopic Dermatitis)

| | | |
|---|---|---|
| FLG | Very High Risk | rs558269137 |
| | High Risk | rs558269137 |
| | Typical Risk | rs558269137 |
| FLG | Very High Risk | rs61816761 |
| | High Risk | rs61816761 |
| | Typical Risk | rs61816761 |
| FLG | Very High Risk | rs150597413 |
| | High Risk | rs150597413 |
| | Typical Risk | rs150597413 |
| FLG | Very High Risk | rs397507563 |
| | High Risk | rs397507563 |
| | Typical Risk | rs397507563 |
| FLG | Very High Risk | rs138726443 |
| | High Risk | rs138726443 |
| | Typical Risk | rs138726443 |
| FLG | Very High Risk | 1249insG HGMD CI083373 |
| | High Risk | 1249insG HGMD CI083373 |
| | Typical Risk | 1249insG HGMD CI083373 |
| FLG | Very High Risk | rs374588791 |
| | High Risk | rs374588791 |
| | Typical Risk | rs374588791 |
| FLG | Very High Risk | rs200519781 |
| | High Risk | rs200519781 |
| | Typical Risk | rs200519781 |
| FLG | Very High Risk | rs121909626 |
| | High Risk | rs121909626 |
| | Typical Risk | rs121909626 |
| FLG | Very High Risk | S2889X HGMD CX082304 |
| | High Risk | S2889X HGMD CX082304 |
| | Typical Risk | S2889X HGMD CX082304 |
| FLG | Very High Risk | rs761212672 |
| | High Risk | rs761212672 |
| | Typical Risk | rs761212672 |

Contact Dermatitis

| | | |
|---|---|---|
| FLG | Increased Risk | rs558269137 |
| | Typical Risk | rs558269137 |
| FLG | Increased Risk | rs61816761 |
| | Typical Risk | rs61816761 |

Psoriasis

| | | |
|---|---|---|
| HLA-C | Very High Risk | rs1265181 |
| | High Risk | rs1265181 |
| | Typical Risk | rs1265181 |
| HLA-C | Very High Risk | rs12191877 |
| | High Risk | rs12191877 |
| | Typical Risk | rs12191877 |
| IL12B | Very High Risk | rs2082412 |
| | High Risk | rs2082412 |
| | Typical Risk | rs2082412 |
| IL23R | Very High Risk | rs2201841 |
| | High Risk | rs2201841 |
| | Typical Risk | rs2201841 |
| TNIP1 | Very High Risk | rs17728338 |
| | High Risk | rs17728338 |
| | Typical Risk | rs17728338 |
| IL13 | Very High Risk | rs20541 |
| | High Risk | rs20541 |
| | Typical Risk | rs20541 |
| TNFAIP3 | Very High Risk | rs610604 |
| | High Risk | rs610604 |
| | Typical Risk | rs610604 |
| MTHFR | Very High Risk | rs1801133 |
| | High Risk | rs1801133 |
| | Typical Risk | rs1801133 |

Rosacea

| | | |
|---|---|---|
| Intergenic between HLA-DRA and | Increased Risk | rs763035 |
| BTNL2 | Typical Risk | rs763035 |
| Intergenic between PRELID2 and | Increased Risk | rs111314066 |
| KCTD16 | Typical Risk | rs111314066 |

Skin Oxidation Protection
Antioxidation Response

| | | |
|---|---|---|
| SOD2 | Decreased Risk | rs4880 |
| | Normal | rs4880 |
| GPX1 | Decreased Risk d | rs1050450 |
| | Normal | rs1050450 |
| CAT | Decreased Risk | rs1001179 |
| | Normal | rs1001179 |
| NQO1 | Decreased Risk | rs1800566 |
| | Normal | rs1800566 |
| NQO1 | Decreased Risk d | rs2917666 |
| | Normal | rs2917666 |

Skin Glycation Risk
Glycation Protection

| | | |
|---|---|---|
| GLO1 | Decreased Risk d | rs1130534 |
| | Normal | rs1130534 |
| GLO1 | Decreased Risk d | rs1049346 |
| | Normal | rs1049346 |
| AGER | Decreased Risk | rs1800624 |
| | Normal | rs1800624 |
| AGER | Decreased Risk | rs1800625 |
| | Normal | rs1800625 |
| AGER | Decreased Risk | rs2070600 |
| | Normal | rs2070600 |

Skin Nutritional Needs
Vitamin A Deficiency

| | | |
|---|---|---|
| BCMO1 | Increased Risk | rs7501331 |
| | Typical Risk | rs7501331 |
| BCMO1 | Increased Risk | rs12934922 |
| | Typical Risk | rs12934922 |

Vitamin B2 Deficiency

| | | |
|---|---|---|
| MTHFR | Increased Risk | rs1801133 |
| | Typical Risk | rs1801133 |

Vitamin B6 Deficiency

| | | |
|---|---|---|
| NBPF3 | Increased Risk | rs4654748 |
| | Typical Risk | rs4654748 |

Vitamin B12 Deficiency

| | | |
|---|---|---|
| FUT2 | Increased Risk | rs602662 |
| | Typical Risk | rs602662 |

Vitamin C Deficiency

| | | |
|---|---|---|
| SLC23A1 | Increased Risk | rs33972313 |
| | Typical Risk | rs33972313 |

Vitamin D Deficiency

| | | |
|---|---|---|
| GC | Increased Risk | rs2282679 |
| | Typical Risk | rs2282679 |

Vitamin E Deficiency

| | | |
|---|---|---|
| Intergenic near APOA5 | Decreased Risk | rs12272004 |
| | Typical Risk | rs12272004 |

Folate-Folic Acid Deficiency

| | | |
|---|---|---|
| MTHFR | Increased Risk | rs1801133 |
| | Typical Risk | rs1801133 |
| MTHFR | Increased Risk | rs1801131 |
| | Typical Risk | rs1801131 |

Omega-3 and Omega-6 Deficiency

| | | |
|---|---|---|
| FADS1 | Increased Risk | rs174547 |
| | Typical Risk | rs174547 |

The foregoing detailed description makes reference to specific exemplary embodiments. However, it will be appreciated that various substitutions, alterations, and/or modifications of the inventive features illustrated herein, and additional applications of the principles illustrated herein, which would occur to one skilled in the relevant art and having possession of this disclosure, can be made to the illustrated embodiments without necessarily departing from the spirit and scope of the invention as defined by the claims, and are to be considered within the scope of this disclosure. Thus, while various aspects and embodiments have been disclosed herein, other aspects and embodiments are contemplated. Similarly, while a number of method steps and components similar or equivalent to those described herein can be used to practice embodiments of the present disclosure, only certain components and method steps are described herein. Furthermore, various well-known aspects of illustrative systems, methods, products, and the like are not described herein in particular detail in order to avoid obscuring aspects of the example embodiments. Such aspects are, however, also contemplated herein.

It will also be appreciated that systems, methods, and/or products according to certain embodiments of the present disclosure may include, incorporate, or otherwise comprise features (e.g., configurations, parameters, properties, steps, components, ingredients, members, elements, parts, and/or portions, etc.) described in other embodiments disclosed and/or described herein. Accordingly, the various features of certain embodiments can be compatible with, combined with, included in, and/or incorporated into other embodiments of the present disclosure. Thus, disclosure of certain features relative to a specific embodiment of the present disclosure should not be construed as limiting application or inclusion of said features to the specific implementation. Rather, it will be appreciated that other embodiments can also include said features without necessarily departing from the scope of the present disclosure. Moreover, unless a feature is described as requiring another feature in combination therewith, any feature herein may be combined with any other feature of a same or different implementation disclosed herein.

Moreover, any steps recited in any method or process described herein and/or recited in the claims can be executed in any suitable order and are not necessarily limited to the order presented in the claims, unless otherwise stated (explicitly or implicitly) in the claims. Such steps can, however, also be required to be performed in any suitable order in certain embodiments of the present disclosure. Accordingly, the scope of the invention should be determined solely by the appended claims and their legal equivalents, rather than by the descriptions and examples given above.

In addition, the present disclosure may be embodied in other specific forms without departing from its spirit or essential characteristics. The described embodiments are to be considered in all respects only as illustrative and not restrictive. The scope of the invention is, therefore, indicated by the appended claims and their legal equivalents rather than by the foregoing description. While certain embodiments and details have been included herein and in the attached disclosure for purposes of illustrating embodiments of the present disclosure, it will be apparent to those skilled in the art that various changes in or to the embodiments disclosed herein may be made without departing from the scope of the disclosure or of the invention, which is defined in the appended claims. The limitations in the claims are to be interpreted broadly based on the language employed in the claims and not limited to specific examples described in the foregoing detailed description, which examples are to be construed as non-exclusive and non-exhaustive. All changes which come within the meaning and range of equivalency of the claims are to be embraced within their scope.

What is claimed is:

1. A method for determining a likelihood of an individual to exhibit skin phenotypic attributes, comprising:
providing a biological sample, the biological sample having a genotype;
determining at least a portion of the genotype and identifying genetic variations associated with skin phenotypic attributes in the portion of the genotype, the skin phenotypic attributes comprising one or more skin nutritional conditions and one or more skin health characteristics, and the genetic variations comprising a first set of preselected genetic variations and a second set of preselected genetic variations, each member of the first set of preselected genetic variations being genetically associated with the one or more skin nutritional conditions and each member of the second set of preselected genetic variations being genetically associated with the one or more skin health characteristics,
wherein the first set of preselected genetic variations comprises biomarkers mapped within one or more genes selected from the group consisting of: SLC23A, MTHFR, NBPF3, FUT2, BCMO1, FADS1, GC genes, and the intergenic region near APOA5, and wherein the second set of preselected genetic variations comprises:
a first subset of preselected genetic variations comprising biomarkers that are genetically associated with skin photoaging and are mapped within one or more genes selected from the group consisting of: MCIR, TYR, SLC45A2 (ATP), SLC24A5, ASIP Region, HERC2, IRF4, EXOC2, STXBP5L, 6p25.3 Region, MMP, and NCOA6, wherein skin photoaging includes including skin aging and skin tone;
a second subset of preselected genetic variations comprising biomarkers that are genetically associated with skin texture and elasticity and are mapped within one or more genes selected from the group consisting of: ACE, HIF1A, ELN, SRPX, HMCN1, TMEM18, and MTHFR;
a third subset of preselected genetic variations comprising biomarkers that are genetically associated with skin moisture factor and are mapped within one or more genes selected from the group consisting of: AQP3 and FLG;
a fourth subset of preselected genetic variations comprising biomarkers that are genetically associated with skin inflammation and allergy and are mapped within one or more genes selected from the group consisting of: FLG, HLA-C, IL12B, IL23R, TNIP1, IL13, MTHFR, the intergenic region between HLA-DRA and BTNL2, the intergenic region between PRELID2 and KCTD16, and TNFAIP3; and
a fifth subset of preselected genetic variations comprising biomarkers that are genetically associated with skin oxidation protection or skin glycation risk and are mapped within one or more genes selected from the group consisting of: SOD2, GPXJ, CAT, NQO1, GLO1, and AGER, and
wherein identifying genetic variations comprises:
identifying a plurality of genetic variations associated with the one or more skin phenotypic attributes;
assigning a weight to each genetic variation of the plurality of genetic variations, the weight comprising an aggregate value of one or more criteria, the one or more criteria selected form the group consisting of: nucleotide sequence homology, expression level, enzyme activity, relative synteny among the preselected biomarkers, family history, ontological relevance, quality of supporting research, and degree of phenotypic significance; and
selecting at least a first and a second genetic variation from the plurality of genetic variations based on the results of weighting each genetic variation, wherein the first genetic variation comprises a member of the first set of preselected genetic variations and the second genetic variation comprises a member of the second set of preselected genetic variations;
generating a personalized biomarker profile for the individual based on the identified genetic variations;
determining the likelihood of the individual to exhibit the skin phenotypic attributes based at least in part upon the personalized biomarker profile and based at least in part on one or more criteria selected from the group consisting of: family history, general medical physiological measures, cholesterol levels, blood pressure, heart rate, growth hormone levels, insulin sensitivity, obesity, body weight, triglyceride levels, red blood cells, bone density, CD scan results, mRNA expression profiles, methylation profiles, protein expression profiles, and enzyme activity; and
generating a personalized genetic profile report that contains genotypic information relevant to the individual's likelihood of exhibiting the one or more skin phenotypic attributes and providing a personalized skin care regimen and a personalized nutritional regimen based on the determined likelihood of the individual to exhibit the one or more skin phenotypic attributes, wherein the personalized skin care regimen and the personalized nutritional regimen comprise one or more selections of adaptive intervention selected from a type and duration of physical exercise, a type and duration of lifestyle counseling, a type and dosing of skin protective products, a type and dosing of skin health medications, a type and dosing of food, and a type and dosing of nutritional supplements.

2. The method of claim 1, wherein at least one of the one or more skin nutritional conditions is selected from the group consisting of: folate level, folic acid level, Vitamin A level, Vitamin B2 level, Vitamin B6 level, Vitamin B12 level, Vitamin B3 level, Vitamin C level, Vitamin D level, Vitamin E level, omega-3 fatty acid level, omega-6 fatty acid level, and combinations thereof.

3. The method of claim 1, wherein biomarker of the second set of preselected genetic variations are selected from the group consisting of: rs1805005, rs2228479, rs885479, rs1805007, rs1805008, rs1805009, rs11547464, rs1110400, rs1805006, rs1393350, rs1126809, rs1042602, rs16891982, rs26722, rs1426654, rs2555364, rs1015362, rs4911414, rs12913832, rs12203592, rs12210050, rs322458, rs1540771, rs1799750, rs4911442, rs1799752, rs4646994, rs11549465, rs7787362, rs35318931, rs10798036, rs7594220, rs1801133, rs1801131, rs558269137, rs17553719, rs61816761, rs150597413, rs397507563, rs12191877, rs2082412, rs2201841, rs17728338, rs20541, rs763035, rs111314066, rs610604, rs138726443, 1249insG (HGMD CI083373), rs374588791 (7264G⁻⁻>T), rs200519781, rs121909626, rs540453626 (8666C⁻⁻>G), rs578153418 (8667C⁻⁻>A), rs761212672 (9887C⁻⁻>A), S2889X (HGMD CX082304), rs4880, rs1050450, rs1001179, rs1800566, rs2917666, rs1130534, rs1049346, rs1800624, rs1800625, rs2070600, and combinations thereof.

4. The method of claim 1, wherein biomarkers of the second set of preselected genetic variations are genetically associated with skin photoaging and are mapped within one or more genes selected from the group consisting of: MCIR, TYR, SLC45A2 (MATP), SLC24A5, ASIP Region, HERC2, IRF4, EXOC2, STXBP5L, 6p25.3 Region, MMP1, and NCOA6, wherein skin photoaging includes including skin aging and skin tone.

5. The method of claim 1, wherein biomarkers of the second set of preselected genetic variations are genetically associated with skin texture and elasticity and are mapped within one or more genes selected from the group consisting of: ACE, HIF1A, ELN, SRPX HMCN1, TEM18, and MTHFR.

6. The method of claim 1, wherein biomarkers of the second set of preselected genetic variations are genetically associated with skin moisture factor and are mapped within one or more genes selected from the group consisting of: AQP3 and FLG.

7. The method of claim 1, wherein biomarkers of the second set of preselected genetic variations are genetically associated with skin inflammation and allergy and are mapped within one or more genes selected from the group consisting of: FLG, HLA-C, IL12B, IL23R, TNIP1, IL13, MTHFR, the intergenic region between HLA-DRA and BTNL2, the intergenic region between PRELID2 and KCTD16, and TNFAIP3.

8. The method of claim 1, wherein biomarkers of the second set of preselected genetic variations are genetically associated with skin oxidation protection or skin glycation risk and are mapped within one or more genes selected from the group consisting of: SOD2, GPX1, CAT, NQO1, GLO1, and AGER.

9. The method of claim 1, wherein at least one of the genetic variations is a nucleic acid based genetic variation selected from the group consisting of: a genetic mutation, a gene amplification, a gene rearrangement, a deletion, an insertion, an InDel mutation, a single nucleotide polymorphism (SNP), an epigenetic alteration, a splicing variant, an RNA/protein overexpression, and an aberrant RNA/protein expression, and combinations thereof.

10. The method of claim 1, wherein the at least a portion of the genotype is determined by performing an analytical assay comprising one or more of nucleic acid sequencing, polypeptide sequencing, restriction digestion, capillary electrophoresis, nucleic acid amplification-based assays, nucleic acid hybridization assay, comparative genomic hybridization, real-time PCR, quantitative reverse transcription PCR (qRT-PCR), PCR-RFLP assay, HPLC, mass-spectrometric genotyping, fluorescent in-situ hybridization (FISH), next generation sequencing (NGS), or a combination thereof.

11. The method of claim 10, wherein the analytical assay is an allele-specific polymerase chain reaction or NGS.

12. The method of claim 1, wherein the at least a portion of the genotype is determined by performing an antibody-based assay comprising one or more of ELISA, immunohistochemistry, western blotting, mass spectrometry, flow cytometry, protein-microarray, immunofluorescence, multiplex detection assay, or combinations thereof.

13. The method of claim 1 further comprising reporting a relative level of risk of exhibiting each of the one or more skin phenotypic attributes, wherein the relative level of risk comprises one of a high risk, an increased risk, a reduced risk, or a normal risk.

14. The method of claim 1, further comprising administering to the individual the selected personalized skin care regimen.

15. A kit for assessing skin health of an individual, comprising:
genotyping reagents, comprising:
a first set of molecular probes specific to a first set of preselected genetic variations, each member of the first set of preselected genetic variations being genetically associated with one or more skin nutritional conditions, wherein the first set of preselected genetic variations comprises biomarkers mapped within one or more genes selected from the group consisting of: SLC23A, MTHFR, NBPF3, FUT2, BCMO1, FADS1, GC genes, and the intergenic region near APOA5, and wherein the second set of preselected genetic variations comprises biomarkers that genetically associate with skin photoaging and are mapped within one or more genes selected from the group consisting of: MCIR, TYR, SLC45A2 (ATP), SLC24A5, ASIP Region, HERC2, IRF4, EXOC2, STXBP5L, 6p25.3 Region, MMP1, and NCOA6, wherein skin photoaging includes including skin aging and skin tone,
wherein the first set of preselected genetic variations comprises biomarkers mapped within one or more genes selected from the group consisting of: SLC23A, MTHFR, NBPF3, FUT2, BCMO1, FADS1, GC genes, and the intergenic region near APOA5, and wherein the second set of preselected genetic variations comprises:
a first subset of preselected genetic variations comprising biomarkers that are genetically associated with skin photoaging and are mapped within one or more genes selected from the group consisting of: MCR, TYR, SLC45A2 (MATP), SLC24A5, ASIP Region, HERC2, IRF4, EXOC2, STXBP5L, 6p25.3 Region, MIP, and NCOA6, wherein skin photoaging includes including skin aging and skin tone;
a second subset of preselected genetic variations comprising biomarkers that are genetically associated with skin texture and elasticity and are mapped within one or more genes selected from the group consisting of: ACE, HIF1A, ELN, SRPX HMCN1, TMEM18, and MTHFR;
a third subset of preselected genetic variations comprising biomarkers that are genetically associated with skin moisture factor and are mapped within one or more genes selected from the group consisting of: AQP3 and FLG;
a fourth subset of preselected genetic variations comprising biomarkers that are genetically associated with skin inflammation and allergy and are mapped within one or more genes selected from the group consisting of: FLG, HLA-C, IL12B, IL23R, TNIP1, IL13, MTHFR, the intergenic region between HLA-DRA and BTNL2, the intergenic region between PRELID2 and KCTD16, and TNFAIP3; and
a fifth subset of preselected genetic variations comprising biomarkers that are genetically associated with skin oxidation protection or skin glycation risk and are mapped within one or more genes selected from the group consisting of: SOD2, GPXJ, CAT, NQO1, GLO1, and AGER; and
a second set of molecular probes specific to a second set of preselected genetic variations, each member of the second set of preselected genetic variations being genetically associated with one or more skin health characteristics.

16. The kit as in claim 15, wherein the first set and the second set of molecular probes are individually selected from the group consisting of: primers, fluorescent oligonucleotide probes, and antibodies.

17. A computer system for generating and displaying a personalized genetics profile, comprising:
one or more processors; and
one or more computer-readable storage media having stored thereon computer-executable instructions that are executable by the one or more processors to cause the computer system to determine the likelihood of an individual to exhibit one or more skin phenotypic attributes, the computer-executable instructions including instructions that are executable to cause the computer system to perform at least the following:
receive sequence data of a user sample, the sequence data comprising at least a portion of a user genotype;
identify a plurality of loci in the sequence data corresponding to a first set of preselected genetic variations and a second set of preselected genetic variations, each member of the first set of preselected genetic variations being genetically associated with one or more skin nutritional conditions and each member of the second set of preselected genetic variations being genetically associated with one or more skin health characteristics;
determine a genotype for each locus of the plurality of loci;
based on one or more criteria associated with the genotype for each locus or for the locus itself, apply a weight to each of the one or more genetic variations corresponding to the genotyped plurality of loci;
calculate a score for at least one of the one or more skin phenotypic attributes based on an aggregated weighted value of genotyped loci corresponding to the at least one of the one or more phenotypic attributes, the score corresponding to the individual's likelihood of exhibiting the at least one of the one or more skin phenotypic attributes; and
generate and display a personalized genetics profile report comprising the one or more genetic variations corresponding to the genotyped plurality of loci and the score for the at least one of the one or more phenotypic attributes.


SEQUENCE LISTING (573 sequences)

SEQ ID NO: 1 - DNA, 25 bp, Artificial Sequence
  Primer rs4646994_II_F1R1_F-sp
tgggaccacaggcgcccgccactac

SEQ ID NO: 2 - DNA, 23 bp, Artificial Sequence
  Primer rs4646994_II_F1R1_R-sp
cgccagccctcccatgcccataa

SEQ ID NO: 3 - DNA, 336 bp, Artificial Sequence
  Amplicon_rs4646994_II_F1R1
  misc_feature (250)..(250): n is a, c, g, or t
  misc_feature (258)..(258): n is a, c, g, or t
  misc_feature (291)..(291): n is a, c, g, or t
tgggaccacaggcgcccgccactacgcccggctaattttttgtatttttagtagagacggggtttcaccgttttagccgggatggtctcgatctcctgacctcgtgatccgcccgcctcggcctcccaaagtgctgggattacaggcgtgatacagtcacttttatgtggtttcgccaattttattccagctctgaaattctctgagctccccttacaagcagaggtgagctaagggctggagctcaagncattcaancccctaccagatctgacgaatgtgatggccacntcccggaaatatgaagacctgttatgggcatgggagggctggcga

SEQ ID NO: 4 - DNA, 20 bp, Artificial Sequence
  Primer rs4646994_WT_F2R2_F-sp
gggactctgtaagccactgc

SEQ ID NO: 5 - DNA, 20 bp, Artificial Sequence
  Primer rs4646994_WT_F2R2_R-sp
ccagcccttagctcacctct

SEQ ID NO: 6 - DNA, 192 bp, Artificial Sequence
  Amplicon rs4646994_WT_F2R2
ctggagagccactcccatcctttctcccatttctctagacctgctgcctatacagtcacttttatgtggtttcgccaattttattccagctctgaaattctctgagctccccttacaagcagaggtgagctaagggctggagctcaaggcattcaaacccctaccagatctgacgaatgtgatggccacgtc

SEQ ID NO: 7 - DNA, 20 bp, Artificial Sequence
  Primer rs4646994_WT_F3R3_F-sp
gtaagccactgctggagagc

SEQ ID NO: 8 - DNA, 20 bp, Artificial Sequence
  Primer rs4646994_WT_F3R3_R-sp
gcttgtaaggggagctcaga

SEQ ID NO: 9 - DNA, 159 bp, Artificial Sequence
  Amplicon rs4646994_WT_F3R3
gggactctgtaagccactgctggagagccactcccatcctttctcccatttctctagacctgctgcctatacagtcacttttatgtggtttcgccaattttattccagctctgaaattctctgagctccccttacaagcagaggtgagctaagggctgg

SEQ ID NO: 10 - DNA, 24 bp, Artificial Sequence
  Primer rs4646994_WT_F1R1_F-sp
ctggagagccactcccatcctttc

SEQ ID NO: 11 - DNA, 25 bp, Artificial Sequence
  Primer rs4646994_WT_F1R1_R-sp
gatgtggccatcacattcgtcagat

SEQ ID NO: 12 - DNA, 131 bp, Artificial Sequence
  Amplicon rs4646994_WT_F1R1
gtaagccactgctggagagccactcccatcctttctcccatttctctagacctgctgcctatacagtcacttttatgtggtttcgccaattttattccagctctgaaattctctgagctccccttacaagc

SEQ ID NO: 13 - DNA, 20 bp, Artificial Sequence
  Primer CI083373_1549_F-sp
ctcttggtggctctgctgtc

SEQ ID NO: 14 - DNA, 23 bp, Artificial Sequence
  Primer CI083373_1549_R-sp
tcaagtccaggagaaagacatgg

SEQ ID NO: 15 - DNA, 225 bp, Artificial Sequence
  Amplicon CI083373_1549
ctcttggtggctctgctgtctcagccyagcctttycgtggcctgacactgattgtgtgtctgagttttctgaatgtccctcactgtcactggcctgactacygctagacccccggtgtccrcgatcgctgactgcagatgaagcttgcccgcgcccagtggctgagtgtctggagctgtctgctgactgctggtggcckgatccatgtctttctcctggacttga

SEQ ID NO: 16 - DNA, 23 bp, Artificial Sequence
  Primer CI083373_t1_1184_F-sp
tgacactgattgtgtgtctgagt

SEQ ID NO: 17 - DNA, 23 bp, Artificial Sequence
  Primer CI083373_t1_1184_R-sp
catgaacaggcaagatcaagtcc

SEQ ID NO: 18 - DNA, 198 bp, Artificial Sequence
  Amplicon CI083373_t1_1184
tgacactgattgtgtgtctgagttttctgaatgtccctcactgtcactggcctgactacygctagacccccggtgtccrcgatcgctgactgcagatgaagcttgcccgcgcccagtggctgagtgtctggagctgtctgctgactgctggtggcckgatccatgtctttctcctggacttgatcttgcctgttcatg

SEQ ID NO: 19 - DNA, 17 bp, Artificial Sequence
  Primer CM081617-r1-1_1925_F-sp
tgactaccactggaccc

SEQ ID NO: 20 - DNA, 18 bp, Artificial Sequence
  Primer CM081617-r1-1_1925_R-sp
tcgtcacgcagagacttc

SEQ ID NO: 21 - DNA, 198 bp, Artificial Sequence
  Amplicon CM081617-r1-1_1925
acgcggaatgcctgagtgtctggagctgtctgctgactgctggtggcgggatccgtgtctctctcctggacttgatcttgcctgttcatgggatgatgcagcctgtccaccagaggaagtctctgcgtgacgagtgcctgattgtctggagcrgtctgcagagtgcccgtgacyggctctgtcttcgtgatgggacct

SEQ ID NO: 22 - DNA, 18 bp, Artificial Sequence
  Primer CM081617_1666_F-sp
acgcggaatgcctgagtg

SEQ ID NO: 23 - DNA, 21 bp, Artificial Sequence
  Primer CM081617_1666_R-sp
aggtcccatcacgaagacaga

SEQ ID NO: 24 - DNA, 189 bp, Artificial Sequence
  Amplicon CM081617_1666
tgactaccactggacccccagtgtctactgtctctgactgcagatgaagcytgtccacgcggaatgcctgagtgtctggagctgtctgctgactgctggtggcgggatccgtgtctctctcctggacttgatcttgcctgttcatgggatgatgcagcctgtccaccagaggaagtctctgcgtgacga

SEQ ID NO: 25 - DNA, 20 bp, Artificial Sequence
  Primer FLG-r3-1_1455_F-sp
gtctggagccatctcttagc

SEQ ID NO: 26 - DNA, 17 bp, Artificial Sequence
  Primer FLG-r3-1_1455_R-sp
ggtcgcgtcaccatgaa

SEQ ID NO: 27 - DNA, 227 bp, Artificial Sequence
  Amplicon FLG-r3-1_1455
atgatggtttctggaagcagacccagaccacctctcagagtcttctgaatgtccctcactgtcactgtcctggctmacactggatccctggcgcctgcttctcctggacccctctgattgtccctggactgcctgtgagtgtctagagatgwcggcatgagtggaagcttcatggtgacgcgaccctgagtgcctggagcygtctcctgattgttcctcattacgtg

SEQ ID NO: 28 - DNA, 22 bp, Artificial Sequence
  Primer FLG-r2-1_1904_F-sp
atgatggtttctggaagcagac

SEQ ID NO: 29 - DNA, 23 bp, Artificial Sequence
  Primer FLG-r2-1_1904_R-sp
cacgtaatgaggaacaatcagga

SEQ ID NO: 30 - DNA, 222 bp, Artificial Sequence
  Primer Amplicon FLG-r2-1_1904
gtctggagccatctcttagctgctcctgagcagatccatgatggtttctggaagcagacccagaccacctctcagagtcttctgaatgtccctcactgtcactgtcctggctcacactggatccctggcgcctgcttctcctggacccctctgattgtccctggactgcctgtgagtgtctagagatgtcggcatgagtggaagcttcatggtgacgcgacc

SEQ ID NO: 31 - DNA, 17 bp, Artificial Sequence
  Primer MC1R-r5-2_1644_F-sp
ccgtcatcctcctgctg

SEQ ID NO: 32 - DNA, 16 bp, Artificial Sequence
  Primer MC1R-r5-2_1644_R-sp
ccagatggccgcaacg

SEQ ID NO: 33 - DNA, 232 bp, Artificial Sequence
  Amplicon MC1R-r5-2_1644
caactccacccccacagccatcccccagctggggctggctgccaaccagacaggagcccggtgcctggaggtgtccatctctgacgggctcytcctcagcctggggctggtgagcttggtggagaacgcgctggtgktggccaccatcgccaagaaccggaacctgcactcacccatgtactgcttcatctgctgcctggccttgtcggamctgctggtgagcgggagcaac

SEQ ID NO: 34 - DNA, 23 bp, Artificial Sequence
  Primer MC1R-r5-2_1645_F-sp
gaccgctacatctccatcttcta

SEQ ID NO: 35 - DNA, 18 bp, Artificial Sequence
  Primer MC1R-r5-2_1645_R-sp
ggccagcatgtggacgta

SEQ ID NO: 36 - DNA, 239 bp, Artificial Sequence
  Amplicon MC1R-r5-2_1645
cactcacccatgtactgcttcatctgctgcctggccttgtcggamctgctggtgagcgggagcaacrtgctggagacggccgtcatcctcctgctggaggccggtgcactggtggcccgggctgcggtgctgcagcagctggacaatgtcaytgacgtgatcacctgcagctccatgctgtccagcctctgcttcctgggcgccatcgccgtggaccgctacatctccatcttctacgc

SEQ ID NO: 37 - DNA, 20 bp, Artificial Sequence
  Primer MC1R-r1-1_2796_F-sp
cactcacccatgtactgctt

SEQ ID NO: 38 - DNA, 22 bp, Artificial Sequence
  Primer MC1R-r1-1_2796_R-sp
gcgtagaagatggagatgtagc

SEQ ID NO: 39 - DNA, 240 bp, Artificial Sequence
  Amplicon MC1R-r1-1_2796
ccagggctttggccttaaaggygctgtcaccctcaccatcctgctgggcattttcttcctctgctggggccccttcttcctgcatctcacactcatcgtcctctgccccgagcaccccacgtgcggctgcatcttcaagaacttcaacctctttctcgccctcatcatctgcaatgccatcatcsaccccctcatctacgccttycacagccaggagctccgcaggacgctcaaggaggt

SEQ ID NO: 40 - DNA, 18 bp, Artificial Sequence
  Primer MC1R-r2-1_1487_F-sp
gctaccacagcatcgtga

SEQ ID NO: 41 - DNA, 17 bp, Artificial Sequence
  Primer MC1R-r2-1_1487_R-sp
cgctgcctcttgtggag

SEQ ID NO: 42 - DNA, 234 bp, Artificial Sequence
  Amplicon MC1R-r2-1_1487
atcatctgcaatgccatcatcsaccccctcatctacgccttycacagccaggagctccgcaggacgctcaaggaggtgctgacrtgctcctggtgagcgcggtgcacrcggctttaagtgtgctgggcagagggaggtggtgatattgtgtggtctggttcctgtgtgaccctgggcagttccttacctccctggtccccgtttgtcaaagaggatggactaaatgatctctga

SEQ ID NO: 43 - DNA, 17 bp, Artificial Sequence
  Primer MC1R-r1-1_1189_F-sp
caactccacccccacag

SEQ ID NO: 44 - DNA, 16 bp, Artificial Sequence
  Primer MC1R-r1-1_1189_R-sp
gttgctcccgctcacc

SEQ ID NO: 45 - DNA, 237 bp, Artificial Sequence
  Amplicon MC1R-r1-1_1189
ctgcatcttcaagaacttcaacctctttctcgccctcatcatctgcaatgccatcatcsaccccctcatctacgccttycacagccaggagctccgcaggacgctcaaggaggtgctgacrtgctcctggtgagcgcggtgcacrcggctttaagtgtgctgggcagagggaggtggtgatattgtgtggtctggttcctgtgtgaccctgggcagttccttacctccctggtcccc

SEQ ID NO: 46 - DNA, 17 bp, Artificial Sequence
  Primer MC1R-r6-1_1770_F-sp
ggctgccaaccagacag

SEQ ID NO: 47 - DNA, 17 bp, Artificial Sequence
  Primer MC1R-r6-1_1770_R-sp
cagcaggtccgacaagg

SEQ ID NO: 48 - DNA, 235 bp, Artificial Sequence
  Amplicon MC1R-r6-1_1770
gctaccacagcatcgtgaycctgmcgygggcgcggcragccgttgcggccatytgggtgkccagtgtcgtcttcagcacgctcttcatcgcctactacgaccacgtggccgtcctgctgtgcctcgtggtcttcttcctggctatgctggtgctcatggccgtgctgtacgtccacatgctggcccgggcctgccagcacgcccagggcatcgcccggctccacaagaggcagcg

SEQ ID NO: 49 - DNA, 17 bp, Artificial Sequence
  Primer MC1R-r1-1_6940_F-sp
ccagggctttggcctta

SEQ ID NO: 50 - DNA, 17 bp, Artificial Sequence
  Primer MC1R-r1-1_6940_R-sp
acctccttgagcgtcct

SEQ ID NO: 51 - DNA, 221 bp, Artificial Sequence
  Amplicon MC1R-r1-1_6940
ccgtcatcctcctgctggaggccggtgcactggtggcccgggctgcggtgctgcagcagctggacaatgtcattgacgtgatcacctgcagctccatgctgtccagcctctgcttcctgggcgccatcgccgtggaccgctacatctccatcttctacgcactgcgctaccacagcatcgtgaccctgccgcgggcgcggcgagccgttgcggccatctgg

SEQ ID NO: 52 - DNA, 23 bp, Artificial Sequence
  Primer MC1R-r1-1_7397_F-sp
ctgcatcttcaagaacttcaacc

SEQ ID NO: 53 - DNA, 17 bp, Artificial Sequence
  Primer MC1R-r1-1_7397_R-sp
ggggaccagggaggtaa

SEQ ID NO: 54 - DNA, 216 bp, Artificial Sequence
  Amplicon MC1R-r1-1_7397
gaccgctacatctccatcttctacgcactgcgctaccacagcatcgtgaccctgccgcgggcgcggcgagccgttgcggccatctgggtggccagtgtcgtcttcagcacgctcttcatcgcctactacgaccacgtggccgtcctgctgtgcctcgtggtcttcttcctggctatgctggtgctcatggccgtgctgtacgtccacatgctggcc

SEQ ID NO: 55 - DNA, 21 bp, Artificial Sequence
  Primer MC1R-r1-1_7232_F-sp
atcatctgcaatgccatcatc

SEQ ID NO: 56 - DNA, 23 bp, Artificial Sequence
  Primer MC1R-r1-1_7232_R-sp
tcagagatcatttagtccatcct

SEQ ID NO: 57 - DNA, 181 bp, Artificial Sequence
  Amplicon MC1R-r1-1_7232
ggctgccaaccagacaggagcccggtgcctggaggtgtccatctctgacgggctcttcctcagcctggggctggtgagcttggtggagaacgcgctggtggtggccaccatcgccaagaaccggaacctgcactcacccatgtactgcttcatctgctgcctggccttgtcggacctgctg

SEQ ID NO: 58 - DNA, 20 bp, Artificial Sequence
  Primer MC1R_CDS_01_F3R3a_F-sp
ctgtccagcctctgcttcct

SEQ ID NO: 59 - DNA, 18 bp, Artificial Sequence
  Primer MC1R_CDS_01_F3R3a_R-sp
ggccagcatgtggacgta

SEQ ID NO: 60 - DNA, 194 bp, Artificial Sequence
  Amplicon MC1R_CDS_01_F3R3a
ccccttcttcctgcatctcacactcatcgtcctctgccccgagcaccccacgtgcggctgcatcttcaagaacttcaacctctttctcgccctcatcatctgcaatgccatcatcsaccccctcatctacgccttycacagccaggagctccgcaggacgctcaaggaggtgctgacrtgctcctggtgagcgc

SEQ ID NO: 61 - DNA, 19 bp, Artificial Sequence
  Primer MC1R_CDS_01_F3R3b_F-sp
gtccagcctctgcttcctg

SEQ ID NO: 62 - DNA, 17 bp, Artificial Sequence
  Primer MC1R_CDS_01_F3R3b_R-sp
cgctgcctcttgtggag

SEQ ID NO: 63 - DNA, 231 bp, Artificial Sequence
  Amplicon MC1R_CDS_01_F3R3b
caactccacccccacagccatcccccagctggggctggctgccaaccagacaggagcccggtgcctggaggtgtccatctctgacgggctcytcctcagcctggggctggtgagcttggtggagaacgcgctggtgktggccaccatcgccaagaaccggaacctgcactcacccatgtactgcttcatctgctgcctggccttgtcggamctgctggtgagcgggagcaa

SEQ ID NO: 64 - DNA, 17 bp, Artificial Sequence
  Primer MC1R_CDS_01_8_F-sp
tgcagctccatgctgtc

SEQ ID NO: 65 - DNA, 21 bp, Artificial Sequence
  Primer MC1R_CDS_01_8_R-sp
catagccaggaagaagaccac

SEQ ID NO: 66 - DNA, 229 bp, Artificial Sequence
  Amplicon MC1R_CDS_01_8
atcgccaagaaccggaacctgcactcacccatgtactgcttcatctgctgcctggccttgtcggamctgctggtgagcgggagcaacrtgctggagacggccgtcatcctcctgctggaggccggtgcactggtggcccgggctgcggtgctgcagcagctggacaatgtcaytgacgtgatcacctgcagctccatgctgtccagcctctgcttcctgggcgccatcg

SEQ ID NO: 67 - DNA, 19 bp, Artificial Sequence
  Primer MC1R_CDS_01_F2R2a_F-sp
ctggtgagcttggtggaga

SEQ ID NO: 68 - DNA, 20 bp, Artificial Sequence
  Primer MC1R_CDS_01_F2R2a_R-sp
atggagatgtagcggtccac

SEQ ID NO: 69 - DNA, 225 bp, Artificial Sequence
  Amplicon MC1R_CDS_01_F2R2a
tgcagctccatgctgtccagcctctgcttcctgggcgccatcgccgtggaccgctacatctccatcttctacgcactgbgctaccacagcatcgtgaycctgmcgygggcgcggcragccgttgcggccatytgggtgkccagtgtcgtcttcagcacgctcttcatcgcctactacgaccacgtggccgtcctgctgtgcctcgtggtcttcttcctggctatg

SEQ ID NO: 70 - DNA, 19 bp, Artificial Sequence
  Primer MC1R_CDS_01_F2R2b_F-sp
gtgagcttggtggagaacg

SEQ ID NO: 71 - DNA, 22 bp, Artificial Sequence
  Primer MC1R_CDS_01_F2R2b_R-sp
cagtgcgtagaagatggagatg

SEQ ID NO: 72 - DNA, 290 bp, Artificial Sequence
  Amplicon MC1R_CDS_01_F2R2b
acctggaggggaagaactgtggggacctggaggcctccaacgactccttcctgcttcctggacaggactatggctgtgcagggatcccagagaagacttctgggctccctcaactccacccccacagccatcccccagctggggctggctgccaaccagacaggagcccggtgcctggaggtgtccatctctgacgggctcttcctcagcctggggctggtgagcttggtggagaacgcgctggtggtggccaccatcgccaagaaccggaacctgcactcacccatgta

SEQ ID NO: 73 - DNA, 19 bp, Artificial Sequence
  Primer MC1R_CDS_01_5_F-sp
atcgccaagaaccggaacc

SEQ ID NO: 74 - DNA, 16 bp, Artificial Sequence
  Primer MC1R_CDS_01_5_R-sp
cgatggcgcccaggaa

SEQ ID NO: 75 - DNA, 284 bp, Artificial Sequence
  Amplicon MC1R_CDS_01_5
agaactgtggggacctggaggcctccaacgactccttcctgcttcctggacaggactatggctgtgcagggatcccagagaagacttctgggctccctcaactccacccccacagccatcccccagctggggctggctgccaaccagacaggagcccggtgcctggaggtgtccatctctgacgggctcttcctcagcctggggctggtgagcttggtggagaacgcgctggtggtggccaccatcgccaagaaccggaacctgcactcacccatgtactgctt

SEQ ID NO: 76 - DNA, 17 bp, Artificial Sequence
  Primer MC1R_CDS_01_3_F-sp
caactccacccccacag

SEQ ID NO: 77 - DNA, 17 bp, Artificial Sequence
  Primer MC1R_CDS_01_3_R-sp
ttgctcccgctcaccag

SEQ ID NO: 78 - DNA, 290 bp, Artificial Sequence
  Amplicon MC1R_CDS_01_3
ctggtgagcttggtggagaacgcgctggtggtggccaccatcgccaagaaccggaacctgcactcacccatgtactgcttcatctgctgcctggccttgtcggacctgctggtgagcgggagcaacgtgctggagacggccgtcatcctcctgctggaggccggtgcactggtggcccgggctgcggtgctgcagcagctggacaatgtcattgacgtgatcacctgcagctccatgctgtccagcctctgcttcctgggcgccatcgccgtggaccgctacatctccat

SEQ ID NO: 79 - DNA, 20 bp, Artificial Sequence
  Primer MC1R_CDS_01_F1R1a_F-sp
acctggaggggaagaactgt

SEQ ID NO: 80 - DNA, 20 bp, Artificial Sequence
  Primer MC1R_CDS_01_F1R1a_R-sp
tacatgggtgagtgcaggtt

SEQ ID NO: 81 - DNA, 300 bp, Artificial Sequence
  Primer Amplicon MC1R_CDS_01_F1R1a
  misc_feature (28)..(28): n is a, c, g, or t
gtgagcttggtggagaacgcgctggtgntggccaccatcgccaagaaccggaacctgcactcacccatgtactgcttcatctgctgcctggccttgtcggacctgctggtgagcgggagcaacgtgctggagacggccgtcatcctcctgctggaggccggtgcactggtggcccgggctgcggtgctgcagcagctggacaatgtcattgacgtgatcacctgcagctccatgctgtccagcctctgcttcctgggcgccatcgccgtggaccgctacatctccatcttctacgcactg

SEQ ID NO: 82 - DNA, 19 bp, Artificial Sequence
  Primer MC1R_CDS_01_F1R1b_F-sp
agaactgtggggacctgga

SEQ ID NO: 83 - DNA, 21 bp, Artificial Sequence
  Primer MC1R_CDS_01_F1R1b_R-sp
aagcagtacatgggtgagtgc

SEQ ID NO: 84 - DNA, 252 bp, Artificial Sequence
  Amplicon MC1R_CDS_01_F1R1b
ctgtccagcctctgcttcctgggcgccatcgccgtggaccgctacatctccatcttctacgcactgcgctaccacagcatcgtgaccctgccgcgggcgcggcgagccgttgcggccatctgggtggccagtgtcgtcttcagcacgctcttcatcgcctactacgaccacgtggccgtcctgctgtgcctcgtggtcttcttcctggctatgctggtgctcatggccgtgctgtacgtccacatgctggcc

SEQ ID NO: 85 - DNA, 21 bp, Artificial Sequence
  Primer MC1R_CDS_01_17_F-sp
ccccttcttcctgcatctcac

SEQ ID NO: 86 - DNA, 16 bp, Artificial Sequence
  Primer MC1R_CDS_01_17_R-sp
gcgctcaccaggagca

SEQ ID NO: 87 - DNA, 300 bp, Artificial Sequence
  Amplicon MC1R_CDS_01_17
gtccagcctctgcttcctgggcgccatcgccgtggaccgctacatctccatcttctacgcactgcgctaccacagcatcgtgaccctgccgcgggcgcggcgagccgttgcggccatctgggtggccagtgtcgtcttcagcacgctcttcatcgcctactacgaccacgtggccgtcctgctgtgcctcgtggtcttcttcctggctatgctggtgctcatggccgtgctgtacgtccacatgctggcccgggcctgccagcacgcccagggcatcgcccggctccacaagaggcagcg

SEQ ID NO: 88 - DNA, 20 bp, Artificial Sequence
  Primer MC1R_CDS_01_F5R5b_F-sp
cccttcttcctgcatctcac

SEQ ID NO: 89 - DNA, 20 bp, Artificial Sequence
  Primer MC1R_CDS_01_F5R5b_R-sp
ccagggaggtaaggaactgc

SEQ ID NO: 90 - DNA, 293 bp, Artificial Sequence
  Amplicon MC1R_CDS_01_F5R5b
cggctgcatcttcaagaacttcaacctctttctcgccctcatcatctgcaatgccatcatcgaccccctcatctacgccttccacagccaggagctccgcaggacgctcaaggaggtgctgacatgctcctggtgagcgcggtgcacgcggctttaagtgtgctgggcagagggaggtggtgatattgtgtggtctggttcctgtgtgaccctgggcagttccttacctccctggtccccgtttgtcaaagaggatggactaaatgatctctgaaagtgttgaagcgcggacc

SEQ ID NO: 91 - DNA, 20 bp, Artificial Sequence
  Primer MC1R_CDS_01_F5R5a_F-sp
cggctgcatcttcaagaact

SEQ ID NO: 92 - DNA, 17 bp, Artificial Sequence
  Primer MC1R_CDS_01_F5R5a_R-sp
kgtccgcgcttcaacac

SEQ ID NO: 93 - DNA, 288 bp, Artificial Sequence
  Amplicon MC1R_CDS_01_F5R5a
cccttcttcctgcatctcacactcatcgtcctctgccccgagcaccccacgtgcggctgcatcttcaagaacttcaacctctttctcgccctcatcatctgcaatgccatcatcgaccccctcatctacgccttccacagccaggagctccgcaggacgctcaaggaggtgctgacatgctcctggtgagcgcggtgcacgcggctttaagtgtgctgggcagagggaggtggtgatattgtgtggtctggttcctgtgtgaccctgggcagttccttacctccctgg

SEQ ID NO: 94 - DNA, 16 bp, Artificial Sequence
  Primer rs1001179_t1_1707_F-sp
gagcgcaaggccccac

SEQ ID NO: 95 - DNA, 21 bp, Artificial Sequence
  Primer rs1001179_t1_1707_R-sp
gaagaccggagatacccagga

SEQ ID NO: 96 - DNA, 221 bp, Artificial Sequence
  Amplicon rs1001179_t1_1707
agcagggtctaagtattccgtctgcaaaactggcaggccaccaacggccgcgtcccagggcggcctgaaggatgctgataaccgggagccccgccctgggttcggctatyccgggcaccccgggccggcggggcgaggctctccaattgctgggccagagcgggacccttcctttccgcaccctcctgggtatctccggtcttcaggcctccttcggagag

SEQ ID NO: 97 - DNA, 23 bp, Artificial Sequence
  Primer rs1001179_1366_F-sp
agcagggtctaagtattccgtct

SEQ ID NO: 98 - DNA, 21 bp, Artificial Sequence
  Primer rs1001179_1366_R-sp
ctctccgaaggaggcctgaag

SEQ ID NO: 99 - DNA, 222 bp, Artificial Sequence
  Amplicon rs1001179_1366
gagcgcaaggccccacccagcagggtctaagtattccgtctgcaaaactggcaggccaccaacggccgcgtcccagggcggcctgaaggatgctgataaccgggagccccgccctgggttcggctatyccgggcaccccgggccggcggggcgaggctctccaattgctgggccagagcgggacccttcctttccgcaccctcctgggtatctccggtcttc

SEQ ID NO: 100 - DNA, 26 bp, Artificial Sequence
  Primer rs1015362-r1-1_1272_F-sp
aattacatatgagtttatgaagagca

SEQ ID NO: 101 - DNA, 24 bp, Artificial Sequence
  Primer rs1015362-r1-1_1272_R-sp
agaagtttcttccacagaaataag

SEQ ID NO: 102 - DNA, 237 bp, Artificial Sequence
  Amplicon rs1015362-r1-1_1272
tgaagagcaggaaagttcttcataatttaaaaaatgtgggataatatttgagatgcttgagaaatgcaaaatgttgctgaacaaatagtcccgaccaggagatgaaaacatctcayactgtttcagacacacagtacacacttaaggaattttggcacaatctcaatttttcaaatgtttcaaacacccytaagcttatttctgtggaagaaacttctttcccacctacgtgcaatc

SEQ ID NO: 103 - DNA, 23 bp, Artificial Sequence
  Primer rs1015362_1438_F-sp
tgaagagcaggaaagttcttcat

SEQ ID NO: 104 - DNA, 22 bp, Artificial Sequence
  Primer rs1015362_1438_R-sp
gattgcacgtaggtgggaaaga

SEQ ID NO: 105 - DNA, 235 bp, Artificial Sequence
  Amplicon rs1015362_1438
aattacatatgagtttatgaagagcaggaaagttcttcataatttaaaaaatgtgggataatatttgagatgcttgagaaatgcaaaatgttgctgaacaaatagtcccgaccaggagatgaaaacatctcayactgtttcagacacacagtacacacttaaggaattttggcacaatctcaatttttcaaatgtttcaaacacccytaagcttatttctgtggaagaaacttct

SEQ ID NO: 106 - DNA, 23 bp, Artificial Sequence
  Primer rs1042602-r2-1_1834_F-sp
aagcataccatcagctcagacta

SEQ ID NO: 107 - DNA, 23 bp, Artificial Sequence
  Primer rs1042602-r2-1_1834_R-sp
gagtctatgccaaggcagaaaag

SEQ ID NO: 108 - DNA, 214 bp, Artificial Sequence
  Amplicon rs1042602-r2-1_1834
  misc_feature (109)..(109): n is a, c, g, or t
gggacctatggccaaatgaaaaatggatcaacacccatgtttaacgacatcaatatttatgacctcwttgtctrgatgcattattatgtgtcaatggatgcactgcttnggggatmtgaaatctggagagacattgattttgcccatgaagcamcarcttttctgccttggcatagactcttcttgwtgbrgtgggaacaagaaatccagaagc

SEQ ID NO: 109 - DNA, 23 bp, Artificial Sequence
  Primer rs1042602_1699_F-sp
gggacctatggccaaatgaaaaa

SEQ ID NO: 110 - DNA, 23 bp, Artificial Sequence
  Primer rs1042602_1699_R-sp
gcttctggatttcttgttcccac

SEQ ID NO: 111 - DNA, 216 bp, Artificial Sequence
  Amplicon rs1042602_1699
  misc_feature (145)..(145): n is a, c, g, or t
aagcataccatcagctcagactatgtcatcccyatagggacctatggccaaatgaaaaatggatcaacacccatgtttaacgacatcaatatttatgacctcwttgtctrgatgcattattatgtgtcaatggatgcactgcttnggggatmtgaaatctggagagacattgattttgcccatgaagcamcarcttttctgccttggcatagactc

SEQ ID NO: 112 - DNA, 18 bp, Artificial Sequence
  Primer rs1049346_1162_F-sp
ccgttcccttcggtcctg

SEQ ID NO: 113 - DNA, 21 bp, Artificial Sequence
  Primer rs1049346_1162_R-sp
ccatcgcgccattcctagtta

SEQ ID NO: 114 - DNA, 210 bp, Artificial Sequence
  Amplicon rs1049346_1162
ccgttcccttcggtcctgtgcccaccttggtactggggtccgcgtcggagcagcaactgagggcggcctcgtccgtgaggccgccggacgggggctgcggttctgccatggctgcrctgcagtatcacagacgacgggacccaaggaacgsaggagtcacccacactacgcctcggccctgtgccgccttaactaggaatggcgcgatgg

SEQ ID NO: 115 - DNA, 19 bp, Artificial Sequence
  Primer rs1049346_2_F-sp
tcctgtgcccaccttggta

SEQ ID NO: 116 - DNA, 20 bp, Artificial Sequence
  Primer rs1049346_2_R-sp
gaggcgtagtgtgggtgact

SEQ ID NO: 117 - DNA, 161 bp, Artificial Sequence
  Amplicon rs1049346_2
tcctgtgcccaccttggtactggggtccgcgtcggagcagcaactgagggcggcctcgtccgtgaggccgccggacgggggctgcggttctgccatggctgcrctgcagtatcacagacgacgggacccaaggaacgsaggagtcacccacactacgcctc

SEQ ID NO: 118 - DNA, 19 bp, Artificial Sequence
  Primer rs1049346_t1_148_F-sp
gtgcccaccttggtactgg

SEQ ID NO: 119 - DNA, 19 bp, Artificial Sequence
  Primer rs1049346_t1_148_R-sp
gctggaagtcagcggtctc

SEQ ID NO: 120 - DNA, 224 bp, Artificial Sequence
  Amplicon rs1049346_t1_148
gtgcccaccttggtactggggtccgcgtcggagcagcaactgagggcggcctcgtccgtgaggccgccggacgggggctgcggttctgccatggctgcrctgcagtatcacagacgacgggacccaaggaacgsaggagtcacccacactacgcctcggccctgtgccgccttaactaggaatggcgcgatggcgccgcgggcgggagaccgctgacttccagc

SEQ ID NO: 121 - DNA, 17 bp, Artificial Sequence
  Primer rs1050450-r2-1_1866_F-sp
gaccagcacccatctcg

SEQ ID NO: 122 - DNA, 20 bp, Artificial Sequence
  Primer rs1050450-r2-1_1866_R-sp
gcttccagaccattgacatc

SEQ ID NO: 123 - DNA, 216 bp, Artificial Sequence
  Amplicon rs1050450-r2-1_1866
  misc_feature (104)..(104): n is a, c, g, or t
cgtaggtttagaggaaacaccctcatagatgaaaacccccccgagacagcagcactgcaactgccaagcagccggggtaggaggggcgccctaggcacagctgngcccttgagacagcagggyttcgatgtcaggctcgatgtcaatggtctggaagcggcggctgtacctgcgtaggggcacaccgtcagggcccaccaggaacttctcaaagtt

SEQ ID NO: 124 - DNA, 23 bp, Artificial Sequence
  Primer rs1050450_1338_F-sp
cgtaggtttagaggaaacaccct

SEQ ID NO: 125 - DNA, 23 bp, Artificial Sequence
  Primer rs1050450_1338_R-sp
aactttgagaagttcctggtggg

SEQ ID NO: 126 - DNA, 211 bp, Artificial Sequence
  Amplicon rs1050450_1338
  misc_feature (157)..(157): n is a, c, g, or t
gaccagcacccatctcgaggkggtattttctgtaagatcaggtgttcctccctcgtaggtttagaggaaacaccctcatagatgaaaacccccccgagacagcagcactgcaactgccaagcagccggggtaggaggggcgccctaggcacagctgngcccttgagacagcagggyttcgatgtcaggctcgatgtcaatggtctggaagc

SEQ ID NO: 127 - DNA, 23 bp, Artificial Sequence
  Primer rs10798036_F1R1_F-sp
tgtcatgaaaaatttctgccttc

SEQ ID NO: 128 - DNA, 20 bp, Artificial Sequence
  Primer rs10798036_F1R1_R-sp
cacatgtgttcctgctctgg

SEQ ID NO: 129 - DNA, 182 bp, Artificial Sequence
  Amplicon rs10798036_F1R1
tggtgtgatatgtagtgagtattggtatctagcagtatttggtgtttgtgtaatagatatttaaaacacttttatctttttaattccsgttcttagcacagtgagtaacacactgtagatgcttaataagtgtccttgaagttttttgaatggataaatctaaccagagcaggaacacatgt

SEQ ID NO: 130 - DNA, 26 bp, Artificial Sequence
  Primer rs10798036_1442_F-sp
tggtgtgatatgtagtgagtattggt

SEQ ID NO: 131 - DNA, 23 bp, Artificial Sequence
  Primer rs10798036_1442_R-sp
acatgtgttcctgctctggttag

SEQ ID NO: 132 - DNA, 243 bp, Artificial Sequence
  Amplicon rs10798036_1442
tgtcatgaaaaatttctgccttctaaaggacttttatgtagtatgataaatattcttctttggtgtgatatgtagtgagtattggtatctagcagtatttggtgtttgtgtaatagatatttaaaacacttttatctttttaattcccgttcttagcacagtgagtaacacactgtagatgcttaataagtgtccttgaagttttttgaatggataaatctaaccagagcaggaacacatgtg

SEQ ID NO: 133 - DNA, 23 bp, Artificial Sequence
  Primer rs10798036-r1-1_566_F-sp
ttggtatctagcagtatttggtg

SEQ ID NO: 134 - DNA, 23 bp, Artificial Sequence
  Primer rs10798036-r1-1_566_R-sp
tcttcctgagaattcataacaca

SEQ ID NO: 135 - DNA, 181 bp, Artificial Sequence
  Amplicon rs10798036-r1-1_566
ttggtatctagcagtatttggtgtttgtgtaatagatatttaaaacacttttatctttttaattccsgttcttagcacagtgagtaacacactgtagatgcttaataagtgtccttgaagttttttgaatggataaatctaaccagagcaggaacacatgtgttatgaattctcaggaaga

SEQ ID NO: 136 - DNA, 23 bp, Artificial Sequence
  Primer rs1110400_2_F-sp
ggaccgctacatctccatcttct

SEQ ID NO: 137 - DNA, 18 bp, Artificial Sequence
  Primer rs1110400_2_R-sp
aagaccacgaggcacagc

SEQ ID NO: 138 - DNA, 165 bp, Artificial Sequence
  Amplicon rs1110400_2
ggaccgctacatctccatcttctacgcactgbgctaccacagcatcgtgaycctgmcgygggcgcggcragccgttgcggccatytgggtgkccagtgtcgtcttcagcacgctcttcatcgcctactacgaccacgtggccgtcctgctgtgcctcgtggtctt

SEQ ID NO: 139 - DNA, 18 bp, Artificial Sequence
  Primer rs1110400_3_F-sp
ccgtggaccgctacatct

SEQ ID NO: 140 - DNA, 21 bp, Artificial Sequence
  Primer rs1110400_3_R-sp
catagccaggaagaagaccac

SEQ ID NO: 141 - DNA, 182 bp, Artificial Sequence
  Amplicon rs1110400_3
ccgtggaccgctacatctccatcttctacgcactgcgctaccacagcatcgtgaccctgccgcgggcgcggcgagccgttgcggccatctgggtggccagtgtcgtcttcagcacgctcttcatcgcctactacgaccacgtggccgtcctgctgtgcctcgtggtcttcttcctggctatg

SEQ ID NO: 142 - DNA, 25 bp, Artificial Sequence
  Primer rs111314066_1_F-sp
tgcatatgttgaaccaactttgaat

SEQ ID NO: 143 - DNA, 24 bp, Artificial Sequence
  Primer rs111314066_1_R-sp
tcctaacattatgtgaaggcagca

SEQ ID NO: 144 - DNA, 200 bp, Artificial Sequence
  Amplicon rs111314066_1
tgcatatgttgaaccaactttgaatcataggaatgttgcctatttgatcatggtatgctgctggatttggtttgctaatattttgttaagaatttttgcatctatgcttatcggtgatactggtctaaagtttcctttttatgttgtgtgtcttccagcttttgttatcaggatgatgctgccttcacataatgttagga

SEQ ID NO: 145 - DNA, 24 bp, Artificial Sequence
  Primer rs111314066_2_F-sp
actttgaatcataggaatgttgcc

SEQ ID NO: 146 - DNA, 23 bp, Artificial Sequence
  Primer rs111314066_2_R-sp
tcatcctgataacaaaagctgga

SEQ ID NO: 147 - DNA, 160 bp, Artificial Sequence
  Amplicon rs111314066_2
actttgaatcataggaatgttgcctatttgatcatggtatgctgctggatttggtttgctaatattttgttaagaatttttgcatctatgcttatcggtgatactggtctaaagtttcctttttatgttgtgtgtcttccagcttttgttatcaggatga

SEQ ID NO: 148 - DNA, 24 bp, Artificial Sequence
  Primer rs111314066_F1R1_F-sp
aaactttttctgggtttatttgga

SEQ ID NO: 149 - DNA, 21 bp, Artificial Sequence
  Primer rs111314066_F1R1_R-sp
caaaagctggaagacacacaa

SEQ ID NO: 150 - DNA, 245 bp, Artificial Sequence
  Amplicon rs111314066_F1R1
aaactttttctgggtttatttggataatcatatggtttttgtcttaaattctgtatatatggggaattacaattgttgatttgcatatgttgaaccaactttgaatcataggaatgttgcctatttgatcatggtatgctgctggatttggtttgctaatattttgttaagaatttttgcatctatgcttatcggtgatactggtctaaagtttcctttttatgttgtgtgtcttccagcttttg

SEQ ID NO: 151 - DNA, 23 bp, Artificial Sequence
  Primer rs1126809_1_F-sp
tctttccatgtctccagatttta

SEQ ID NO: 152 - DNA, 19 bp, Artificial Sequence
  Primer rs1126809_1_R-sp
aatgggtgcattggcttct

SEQ ID NO: 153 - DNA, 181 bp, Artificial Sequence
  Amplicon rs1126809_1
  misc_feature (41)..(41): n is a, c, g, or t
tctttccatgtctccagattttaatatatgccttattttantttaaaaattttcaaatgtttcttttatacacaatatgtttcttagtctgaataaccttttcctctgcagtatttttgagcagtkgctcyraagkcmccgtcytcttcaagaagtttatccagaagccaatgcacccatt

SEQ ID NO: 154 - DNA, 25 bp, Artificial Sequence
  Primer rs1126809-r1-1_1631_F-sp
tgtttcttagtctgaataacctttt

SEQ ID NO: 155 - DNA, 26 bp, Artificial Sequence
  Primer rs1126809-r1-1_1631_R-sp
ctttggatgaaataaagaaatcacca

SEQ ID NO: 156 - DNA, 262 bp, Artificial Sequence
  Amplicon rs1126809-r1-1_1631
tttcttagtctgaataaccttttcctctgcagtatttttgagcagtggctccgaaggcaccgtcctcttcaagaagtttatccagaagccaatgcacccattggacataaccgggaatcctacatggttccttttataccactgtacagaaatggtgatttctttatttcatccaaagatctgggctatgactatagctatctacaagattcaggtaaagtttactttctttcagaggaattgctgaatctagtgttaccaa

SEQ ID NO: 157 - DNA, 25 bp, Artificial Sequence
  Primer rs1126809_F1R1_F-sp
tttcttagtctgaataaccttttcc

SEQ ID NO: 158 - DNA, 23 bp, Artificial Sequence
  Primer rs1126809_F1R1_R-sp
ttggtaacactagattcagcaat

SEQ ID NO: 159 - DNA, 180 bp, Artificial Sequence
  Amplicon rs1126809_F1R1
tgtttcttagtctgaataaccttttcctctgcagtatttttgagcagtkgctcyraagkcmccgtcytcttcaagaagtttatccagaagccaatgcacccattrgacataaccrggaatcctacatggttccttttataccactgtacagaaatggtgatttctttatttcatccaaag

SEQ ID NO: 160 - DNA, 23 bp, Artificial Sequence
  Primer rs1130534_F1R1_F-sp
aaatcatggtgagatggtaagtg

SEQ ID NO: 161 - DNA, 20 bp, Artificial Sequence
  Primer rs1130534_F1R1_R-sp
aattggggcactgaagatga

SEQ ID NO: 162 - DNA, 287 bp, Artificial Sequence
  Amplicon rs1130534_F1R1
aaatcatggtgagatggtaagtgtaattttagatcgagactctgaaattaaacaactcctaaataagattatttgtggaaaataaaaataatcatatctctaataaattgatcatacttaattaggacattaatgttttaggtttattaaaatcataattatatacataatacaaaatcacacaaatctacatatatcacataaaaacaggcaaacttaccgaatcctcgagggtctgaattgccattgtggtaactctgggtctcatcatcttcagtgccccaatt

SEQ ID NO: 163 - DNA, 26 bp, Artificial Sequence
  Primer rs1130534-r2-1_1736_F-sp
aatcacacaaatctacatatatcaca

SEQ ID NO: 164 - DNA, 23 bp, Artificial Sequence
  Primer rs1130534-r2-1_1736_R-sp
caaagatgttacttgcttttcag

SEQ ID NO: 165 - DNA, 185 bp, Artificial Sequence
  Amplicon rs1130534-r2-1_1736
aatcacacaaatctacatatatcacataaaaacaggcaaacttaccgaawcctcgagggtctgaattgccattgtggtaactctgggtckcatcatcttcagtgccccaattgctacaaaaggaaggaaaacatagctacaatgtcctagggaatagatattctgaaaagcaagtaacatctttg

SEQ ID NO: 166 - DNA, 25 bp, Artificial Sequence
  Primer rs1130534-r2-1_7_F-sp
tatatcacataaaaacaggcaaact

SEQ ID NO: 167 - DNA, 23 bp, Artificial Sequence
  Primer rs1130534-r2-1_7_R-sp
acgtggtaagatttcaaagatgt

SEQ ID NO: 168 - DNA, 182 bp, Artificial Sequence
  Amplicon rs1130534-r2-1_7
tatatcacataaaaacaggcaaacttaccgaawcctcgagggtctgaattgccattgtggtaactctgggtckcatcatcttcagtgccccaattgctacaaaaggaaggaaaacatagctacaatgtcctagggaatagatattctgaaaagcaagtaacatctttgaaatcttaccacgt

SEQ ID NO: 169 - DNA, 18 bp, Artificial Sequence
  Primer rs11547464_rs1805007_F1R1_F-sp
ccatgctgtccagcctct

SEQ ID NO: 170 - DNA, 20 bp, Artificial Sequence
  Primer rs11547464_rs1805007_F1R1_R-sp
ccagcatagccaggaagaag

SEQ ID NO: 171 - DNA, 162 bp, Artificial Sequence
  Amplicon rs11547464_rs1805007_F1R1
tgcagctccatgctgtccagcctctgcttcctgggcgccatcgccgtggaccgctacatctccatcttctacgcactgbgctaccacagcatcgtgaycctgmcgygggcgcggcragccgttgcggccatytgggtgkccagtgtcgtcttcagcacgctc

SEQ ID NO: 172 - DNA, 18 bp, Artificial Sequence
  Primer rs11547464_2_F-sp
tgcagctccatgctgtcc

SEQ ID NO: 173 - DNA, 20 bp, Artificial Sequence
  Primer rs11547464_2_R-sp
gagcgtgctgaagacgacac

SEQ ID NO: 174 - DNA, 222 bp, Artificial Sequence
  Amplicon rs11547464_2
ccatgctgtccagcctctgcttcctgggcgccatcgccgtggaccgctacatctccatcttctacgcactgcgctaccacagcatcgtgaccctgccgcgggcgcggcgagccgttgcggccatctgggtggccagtgtcgtcttcagcacgctcttcatcgcctactacgaccacgtggccgtcctgctgtgcctcgtggtcttcttcctggctatgctgg

SEQ ID NO: 175 - DNA, 17 bp, Artificial Sequence
  Primer rs11547464-r2-1_3_F-sp
ccgtcatcctcctgctg

SEQ ID NO: 176 - DNA, 18 bp, Artificial Sequence
  Primer rs11547464-r2-1_3_R-sp
tcacgatgctgtggtagc

SEQ ID NO: 177 - DNA, 183 bp, Artificial Sequence
  Amplicon rs11547464-r2-1_3
ccgtcatcctcctgctggaggccggtgcactggtggcccgggctgcggtgctgcagcagctggacaatgtcaytgacgtgatcacctgcagctccatgctgtccagcctctgcttcctgggcgccatcgccgtggaccgctacatctccatcttctacgcactgbgctaccacagcatcgtga

SEQ ID NO: 178 - DNA, 24 bp, Artificial Sequence
  Primer rs11549465_1422_F-sp
ggtgtggccattgtaaaaactcat

SEQ ID NO: 179 - DNA, 23 bp, Artificial Sequence
  Primer rs11549465_1422_R-sp
ggtggcattagcagtaggttctt

SEQ ID NO: 180 - DNA, 228 bp, Artificial Sequence
  Primer Amplicon rs11549465_1422
ggtgtggccattgtaaaaactcatgtatttgctgttttaaaggacacagatttagacttggagatgttagctccctatatcccaatggatgatgacttccagttacgttccttcgatcagttgtcaycattagaaagcagttccrcaagccctgaaagcgcaagtcctcaaagcacagttacagtattccagcagactcaaatacaagaacctactgctaatgccacc

SEQ ID NO: 181 - DNA, 26 bp, Artificial Sequence
  Primer rs11549465_t1_1972_F-sp
ggacacagatttagacttggagatgt

SEQ ID NO: 182 - DNA, 23 bp, Artificial Sequence
  Primer rs11549465_t1_1972_R-sp
attcatcagtggtggcagtggta

SEQ ID NO: 183 - DNA, 212 bp, Artificial Sequence
  Amplicon rs11549465_t1_1972
ggacacagatttagacttggagatgttagctccctatatcccaatggatgatgacttccagttacgttccttcgatcagttgtcaycattagaaagcagttccrcaagccctgaaagcgcaagtcctcaaagcacagttacagtattccagcagactcaaatacaagaacctactgctaatgccaccactaccactgccaccactgatgaat

SEQ ID NO: 184 - DNA, 22 bp, Artificial Sequence
  Primer rs121909626_F2R2_F-sp
ggtgtctggagccatctcttag

SEQ ID NO: 185 - DNA, 21 bp, Artificial Sequence
  Primer rs121909626_F2R2_R-sp
gtgcaagcagacaaactcgta

SEQ ID NO: 186 - DNA, 234 bp, Artificial Sequence
  Amplicon rs121909626_F2R2
gatggtttctggaagcagacccagaccacctctcagagtcttctgagtgtccctsactgtcactgtcctggctaaaactggatccccagttyctgcttktcctgggcccctctsattgtccctggcccacctgcgagtgtcyagagctgtcggcccgagaggaagcttcatgrtgacgcgaccctgagtgcctggagccrtctcctgattgttcatcgttacgagtttgtctgc

SEQ ID NO: 187 - DNA, 22 bp, Artificial Sequence
  Primer rs121909626_1817_F-sp
gatggtttctggaagcagaccc

SEQ ID NO: 188 - DNA, 23 bp, Artificial Sequence
  Primer rs121909626_1817_R-sp
gcagacaaactcgtaacgatgaa

SEQ ID NO: 189 - DNA, 170 bp, Artificial Sequence
  Amplicon rs121909626_1817
aactggatccccagttcctgcttgtcctgggcccctctgattgtccctggcccacctgcgagtgtccagagctgtcggcccgagaggaagcttcatggtgacgcgaccctgagtgcctggagccgtctcctgattgttcatcgttacgagtttgtctgcttgcacttctg

SEQ ID NO: 190 - DNA, 20 bp, Artificial Sequence
  Primer rs121909626_F1R1_F-sp
aactggatccccagttcctg

SEQ ID NO: 191 - DNA, 22 bp, Artificial Sequence
  Primer rs121909626_F1R1_R-sp
cagaagtgcaagcagacaaact

SEQ ID NO: 192 - DNA, 282 bp, Artificial Sequence
  Amplicon rs121909626_F1R1
ggtgtctggagccatctcttagctgctcctgagcagatccatgatggtttctggaagcagacccagaccacctctcagagtcttctgagtgtccctgactgtcactgtcctggctaaaactggatccccagttcctgcttgtcctgggcccctctgattgtccctggcccacctgcgagtgtccagagctgtcggcccgagaggaagcttcatggtgacgcgaccctgagtgcctggagccgtctcctgattgttcatcgttacgagtttgtctgcttgcac

SEQ ID NO: 193 - DNA, 24 bp, Artificial Sequence
  Primer rs12191877_1593_F-sp
tctcagtctctcattccttcttca

SEQ ID NO: 194 - DNA, 23 bp, Artificial Sequence
  Primer rs12191877_1593_R-sp
tagtttggcacacacatacatgc

SEQ ID NO: 195 - DNA, 194 bp, Artificial Sequence
  Amplicon rs12191877_1593
tctcagtctctcattccttcttcatttattatctgaataatttctaagtaagagattcaccctcctctactgtttgtttactaytagaaacttgstttttgagagactaasccaatcatctactcayctatgatccagcaatagcactcttakttctaaaccaatagaaatgcatgtatgtgtgtgccaaacta

SEQ ID NO: 196 - DNA, 23 bp, Artificial Sequence
  Primer rs12191877-r2-1_309_F-sp
agattcaccctcctctactgttt

SEQ ID NO: 197 - DNA, 23 bp, Artificial Sequence
  Primer rs12191877-r2-1_309_R-sp
ctcacatttcttgtcctttcact

SEQ ID NO: 198 - DNA, 230 bp, Artificial Sequence
  Amplicon rs12191877-r2-1_309
agattcaccctcctctactgtttgtttactaytagaaacttgstttttgagagactaasccaatcatctactcayctatgatccagcaatagcactcttakttctaaaccaatagaaatgcatgtatgtgtgtgccaaactatatgaaaatattattcatagcagcacgatttgtaaaatctgkatacaacaaaattgtctatcaacagtgaaaggacaagaaatgtgag

SEQ ID NO: 199 - DNA, 25 bp, Artificial Sequence
  Primer rs12203592_1305_F-sp
tggaagattttggaagtagtgcctt

SEQ ID NO: 200 - DNA, 24 bp, Artificial Sequence
  Primer rs12203592_1305_R-sp
agtgaaaacactcatggaggatca

SEQ ID NO: 201 - DNA, 228 bp, Artificial Sequence
  Amplicon rs12203592_1305
tggaagattttggaagtagtgccttatcatgtgaaaccacagggcagctgatctcttcaggctttcttgatgtgaatgacagctttgtttcatccactttggtgggtaaaagaaggyaaattcccctgtggtacttttggtgccaggtttagccatatgacgaagctttacataaaacagtacaagtatctccattgtcctttatgatcctccatgagtgttttcact

SEQ ID NO: 202 - DNA, 23 bp, Artificial Sequence
  Primer rs12203592_t1_1408_F-sp
ccttatcatgtgaaaccacaggg

SEQ ID NO: 203 - DNA, 24 bp, Artificial Sequence
  Primer rs12203592_t1_1408_R-sp
tggagtgaacccttcatcagacta

SEQ ID NO: 204 - DNA, 231 bp, Artificial Sequence
  Amplicon rs12203592_t1_1408
ccttatcatgtgaaaccacagggcagctgatctcttcaggctttcttgatgtgaatgacagctttgtttcatccactttggtgggtaaaagaaggyaaattcccctgtggtacttttggtgccaggtttagccatatgacgaagctttacataaaacagtacaagtatctccattgtcctttatgatcctccatgagtgttttcacttagtctgatgaagggttcactcca

SEQ ID NO: 205 - DNA, 23 bp, Artificial Sequence
  Primer rs12210050_1985_F-sp
gatgagggctccgtaaatccata

SEQ ID NO: 206 - DNA, 23 bp, Artificial Sequence
  Primer rs12210050_1985_R-sp
cttatcttcacaggcacaggaca

SEQ ID NO: 207 - DNA, 197 bp, Artificial Sequence
  Amplicon rs12210050_1985
gatgagggctccgtaaatccatatgctgccatggaaagaacggggcagacggacggtcacctatgcgttcctctcctgctcagcgagcatgcttttgacaggaggaagagcaaatggggcagtcaygcacgcatttgtctcgggtgagggagaaatgacgtccagtcctgcctttgtcctgtgcctgtgaagataag

SEQ ID NO: 208 - DNA, 22 bp, Artificial Sequence
  Primer rs12210050-r1-1_148_F-sp
catatgctgccatggaaagaac

SEQ ID NO: 209 - DNA, 21 bp, Artificial Sequence
  Primer rs12210050-r1-1_148_R-sp
tctgttgaatttcaccctgac

SEQ ID NO: 210 - DNA, 214 bp, Artificial Sequence
  Amplicon rs12210050-r1-1_148
catatgctgccatggaaagaacggggcagacggacggtcacctatgcgttcctctcctgctcagcgagcatgcttttgacaggaggaagagcaaatggggcagtcaygcacgcatttgtctcgggtgagggagaaatgacgtccagtcctgcctttgtcctgtgcctgtgaagataagctgtgaattcacattgtcagggtgaaattcaacaga

SEQ ID NO: 211 - DNA, 22 bp, Artificial Sequence
  Primer rs12272004_t1_190_F-sp
ctatgactccatgtgtccagca

SEQ ID NO: 212 - DNA, 24 bp, Artificial Sequence
  Primer rs12272004_t1_190_R-sp
cgtgattttgtaattcaggctagg

SEQ ID NO: 213 - DNA, 214 bp, Artificial Sequence
  Amplicon rs12272004_t1_190
tacactgagcggtctgtatcggtccatccccaytgcagccgggcagccccatccarcttgccttgtaaggctcggggtgtggcttcctgtccttctgcaaagmccacctaagtcacaaagcccctctttcccctttaccctacttttcattcgcttttctccgaatgactccctagcctgaattacaaaatcacgaagacatttcacaaacccc

SEQ ID NO: 214 - DNA, 22 bp, Artificial Sequence
  Primer rs12272004_1492_F-sp
tacactgagcggtctgtatcgg

SEQ ID NO: 215 - DNA, 22 bp, Artificial Sequence
  Primer rs12272004_1492_R-sp
ggggtttgtgaaatgtcttcgt

SEQ ID NO: 216 - DNA, 223 bp, Artificial Sequence
  Amplicon rs12272004_1492
ctatgactccatgtgtccagcaggtggctacactgagcggtctgtatcggtccatccccaytgcagccgggcagccccatccarcttgccttgtaaggctcggggtgtggcttcctgtccttctgcaaagmccacctaagtcacaaagcccctctttcccctttaccctacttttcattcgcttttctccgaatgactccctagcctgaattacaaaatcacg

SEQ ID NO: 217 - DNA, 22 bp, Artificial Sequence
  Primer rs1265181_F1R1_F-sp
cagcagcctattcacctaacaa

SEQ ID NO: 218 - DNA, 22 bp, Artificial Sequence
  Primer rs1265181_F1R1_R-sp
tccaaaggagaggataaacagg

SEQ ID NO: 219 - DNA, 227 bp, Artificial Sequence
  Amplicon rs1265181_F1R1
cagcagcctattcacctaacaataaactgagtgcccattatgtgccaagaactggagataaggatatgagtaaggaatcttacttatctccagttcttatagcatatactcattctgtttctctttcctttggcctagtttgagtgcccagcaggtgtttcaagtcactgattacgtatctactctgcgaaagttgtttgtgcagcctgtttatcctctcctttgga

SEQ ID NO: 220 - DNA, 22 bp, Artificial Sequence
  Primer rs1265181_F1R1b_F-sp
cagcagcctattcacctaacaa

SEQ ID NO: 221 - DNA, 20 bp, Artificial Sequence
  Primer rs1265181_F1R1b_R-sp
aaacaggctgcacaaacaac

SEQ ID NO: 222 - DNA, 212 bp, Artificial Sequence
  Amplicon rs1265181_F1R1b
cagcagcctattcacctaacaataaactgagtgcccattatgtgccaagaactggagataaggatatgagtaaggaatcttacttatctccagttcttatagcatatactcattctgtttctctttcctttggcctagtttgagtgcccagcaggtgtttcaagtcactgattacgtatctactctgcgaaagttgtttgtgcagcctgttt

SEQ ID NO: 223 - DNA, 21 bp, Artificial Sequence
  Primer rs1265181_F2R2_F-sp
tcctttggcctagtttgagtg

SEQ ID NO: 224 - DNA, 18 bp, Artificial Sequence
  Primer rs1265181_F2R2_R-sp
gggaagcagagcttgcag

SEQ ID NO: 225 - DNA, 203 bp, Artificial Sequence
  Amplicon rs1265181_F2R2
tcctttggcctagtttgagtgcccagcaggtgtttcaagtcactgattacgtatctactctgcgaaagttgtttgtgcagcctgtttatcctctcctttggaacttcagtactcttttttttttttttgagacggagtcttgctctgttgcccaggctagagttcagtggtgtgatctcggctcactgcaagctctgcttccc

SEQ ID NO: 226 - DNA, 23 bp, Artificial Sequence
  Primer rs12913832-r1-1_181_F-sp
agtcttgtaatcaacatcagggt

SEQ ID NO: 227 - DNA, 24 bp, Artificial Sequence
  Primer rs12913832-r1-1_181_R-sp
tctaaatgtatactgcttcaagtg

SEQ ID NO: 228 - DNA, 234 bp, Artificial Sequence
  Amplicon rs12913832-r1-1_181
agtcttgtaatcaacatcagggtaaaaatcatgtgttaatacaaaggtacaggaacaaagaatttgttcttcatggctctctgtgtctgatccaagaggcgaggccagtttcatttgagcattaartgtcaagttctgcacgctatcatcatcaggggccgaggcttctctttgtttttaattaattgtttttaactgtgagtttatatacacttgaagcagtatacatttaga

SEQ ID NO: 229 - DNA, 23 bp, Artificial Sequence
  Primer rs12913832-r2-1_754_F-sp
acaaaggtacaggaacaaagaat

SEQ ID NO: 230 - DNA, 23 bp, Artificial Sequence
  Primer rs12913832-r2-1_754_R-sp
actgtctcatgggtagtaatcaa

SEQ ID NO: 231 - DNA, 238 bp, Artificial Sequence
  Amplicon rs12913832-r2-1_754
acaaaggtacaggaacaaagaatttgttcttcatggctctctgtgtctgatccaagaggcgaggccagtttcatttgagcattaartgtcaagttctgcacgctatcatcatcaggggccgaggcttctctttgtttttaattaattgtttttaactgtgagtttatatacacttgaagcagtatacatttagaaatggtmtacttgtcgtttctttgattactacccatgagacagt

SEQ ID NO: 232 - DNA, 19 bp, Artificial Sequence
  Primer rs12934922_t1_1228_F-sp
cctgctctccccaagctac

SEQ ID NO: 233 - DNA, 18 bp, Artificial Sequence
  Primer rs12934922_t1_1228_R-sp
gcctccctccatggtcag

SEQ ID NO: 234 - DNA, 212 bp, Artificial Sequence
  Amplicon rs12934922_t1_1228
gctttggagtcaccgagaactatgtcatcttccttgagcagcctttcaggttggatattctcaagatggcaaccgcatacatccggagwatgagctgggcctcctgcctggctttccacagggaggagaaggtgaggtctggctggactctagcccagtgggtgctggctgaccatggagggaggctggtgtgcaggagggtgaagtttaag

SEQ ID NO: 235 - DNA, 23 bp, Artificial Sequence
  Primer rs12934922_1546_F-sp
gctttggagtcaccgagaactat

SEQ ID NO: 236 - DNA, 23 bp, Artificial Sequence
  Primer rs12934922_1546_R-sp
cttaaacttcaccctcctgcaca

SEQ ID NO: 237 - DNA, 212 bp, Artificial Sequence
  Amplicon rs12934922_1546
cctgctctccccaagctactmccacagctttggagtcaccgagaactatgtcatcttccttgagcagcctttcaggttggatattctcaagatggcaaccgcatacatccggagwatgagctgggcctcctgcctggctttccacagggaggagaaggtgaggtctggctggactctagcccagtgggtgctggctgaccatggagggaggc

SEQ ID NO: 238 - DNA, 20 bp, Artificial Sequence
  Primer rs138726443_1592_F-sp
ccttcctccactgcttgacc

SEQ ID NO: 239 - DNA, 23 bp, Artificial Sequence
  Primer rs138726443_1592_R-sp
gacgttcagggtctttcctctac

SEQ ID NO: 240 - DNA, 194 bp, Artificial Sequence
  Amplicon rs138726443_1592
ccttcctccactgcttgacccygggtgtccatgaatggtgtcctgaccctcttgggacgttgagtgcctggagctgtctcgtgcctgctygtggtgggatccttgtcttcctccagtgctggtcccggtccgtccatgggcggactcagactgttcatgagtgctcayctggtagaggaaagaccctgaacgtc

SEQ ID NO: 241 - DNA, 24 bp, Artificial Sequence
  Primer rs1393350_1389_F-sp
gcttggatttccagacatcatcta

SEQ ID NO: 242 - DNA, 23 bp, Artificial Sequence
  Primer rs1393350_1389_R-sp
agaggccatgttagggagatttg

SEQ ID NO: 243 - DNA, 234 bp, Artificial Sequence
  Amplicon rs1393350_1389
gcttggatttccagacatcatctattttcctggacatcattttctcactgatttctttatccccctgatgcgtgcatatccaccaactcctactcttcctcagtcccttctctgcaacraaatctgtgtggtcttttacaaatgataatctgttcgtgttatcattcaccttcccagatacaaacagtgactttctgttaggaaaaaaacacaaatctccctaacatggcctct

SEQ ID NO: 244 - DNA, 25 bp, Artificial Sequence
  Primer rs1393350_t1_1176_F-sp
tctcactgatttctttatccccctg

SEQ ID NO: 245 - DNA, 23 bp, Artificial Sequence
  Primer rs1393350_t1_1176_R-sp
gccaaatcatgaagggtagcaga

SEQ ID NO: 246 - DNA, 212 bp, Artificial Sequence
  Amplicon rs1393350_t1_1176
tctcactgatttctttatccccctgatgcgtgcatatccaccaactcctactcttcctcagtcccttctctgcaacraaatctgtgtggtcttttacaaatgataatctgttcgtgttatcattcaccttcccagatacaaacagtgactttctgttaggaaaaaaacacaaatctccctaacatggcctctgctacccttcatgatttggc

SEQ ID NO: 247 - DNA, 26 bp, Artificial Sequence
  Primer rs1426654_1665_F-sp
gctgtagctttgagtatctattgtgt

SEQ ID NO: 248 - DNA, 23 bp, Artificial Sequence
  Primer rs1426654_1665_R-sp
atcacactgagtaagcaagaagt

SEQ ID NO: 249 - DNA, 213 bp, Artificial Sequence
  Amplicon rs1426654_1665
gctgtagctttgagtatctattgtgtttagttgtaaagacatactctttcactttattaggcataacaatcatttcatttatgttcagcccttggattgtctcaggatgttgcaggcrcaactttcatggcagcgggcagttcagctcctgaattagttactgctttcctaggtaaatattgctccttatacttcttgcttactcagtgtgat

SEQ ID NO: 250 - DNA, 26 bp, Artificial Sequence
  Primer rs1426654-r2-1_1494_F-sp
agacatactctttcactttattaggc

SEQ ID NO: 251 - DNA, 23 bp, Artificial Sequence
  Primer rs1426654-r2-1_1494_R-sp
acctatgtgcaagatagtaccag

SEQ ID NO: 252 - DNA, 231 bp, Artificial Sequence
  Amplicon rs1426654-r2-1_1494
agacatactctttcactttattaggcataacaatcatttcatttatgttcagcccttggattgtctcaggatgttgcaggcrcaactttcatggcagcgggcagttcagctcctgaattagttactgctttcctaggtaaatattgctccttatacttcttgcttactcagtgtgatttttattttcttcaagttaacactaacttagctggtactatcttgcacataggt

SEQ ID NO: 253 - DNA, 21 bp, Artificial Sequence
  Primer rs150597413_1334_F-sp
ccagaggaagtctctgcgtga

SEQ ID NO: 254 - DNA, 16 bp, Artificial Sequence
  Primer rs150597413_1334_R-sp
gaagcaggcgccaggg

SEQ ID NO: 255 - DNA, 232 bp, Artificial Sequence
  Amplicon rs150597413_1334
ccagaggaagtctctgcgtgacgagtgcctgattgtctggagcrgtctgcagagtgcccgtgacyggctctgtcttcgtgatgggacctggggtgtctggagccgtgccttgactgctcctgaacagatccacgatggtttctggaagcagacccagaccacctctcagagtcttctgaatgtccctcactgtcactgtcctggctmacactggatccctggcgcctgcttc

SEQ ID NO: 256 - DNA, 21 bp, Artificial Sequence
  Primer rs150597413_t1_175_F-sp
cgtgacgagtgcctgattgtc

SEQ ID NO: 257 - DNA, 19 bp, Artificial Sequence
  Primer rs150597413_t1_175_R-sp
cagaggggtccaggagaag

SEQ ID NO: 258 - DNA, 231 bp, Artificial Sequence
  Amplicon rs150597413_t1_175
cgtgacgagtgcctgattgtctggagcrgtctgcagagtgcccgtgacyggctctgtcttcgtgatgggacctggggtgtctggagccgtgccttgactgctcctgaacagatccacgatggtttctggaagcagacccagaccacctctcagagtcttctgaatgtccctcactgtcactgtcctggctmacactggatccctggcgcctgcttctcctggacccctctg

SEQ ID NO: 259 - DNA, 23 bp, Artificial Sequence
  Primer rs1540771_1247_F-sp
atgacagtaacctgtggacatct

SEQ ID NO: 260 - DNA, 20 bp, Artificial Sequence
  Primer rs1540771_1247_R-sp
cctgcccacattccttcagc

SEQ ID NO: 261 - DNA, 240 bp, Artificial Sequence
  Amplicon rs1540771_1247
  misc_feature (121)..(121): n is a, c, g, or t
atgacagtaacctgtggacatctaygtctsgtatcctctgaaggaaggaaatgaggcaagcttggcagtttatgrtagaagagagaggagggtttctgtgttatgaactgcaygagttggntgagctcagtctatcacgtgtgtggtgggcacatggccagactccatgtgccctggacttgagtgtggtcttcagtgtgggggcatggggctgagccaggctgaaggaatgtgggcagg

SEQ ID NO: 262 - DNA, 23 bp, Artificial Sequence
  Primer rs1540771_t1_192_F-sp
tcctctgaaggaaggaaatgagg

SEQ ID NO: 263 - DNA, 17 bp, Artificial Sequence
  Primer rs1540771_t1_192_R-sp
ctggctcagccccatgc

SEQ ID NO: 264 - DNA, 187 bp, Artificial Sequence
  Amplicon rs1540771_t1_192
  misc_feature (88)..(88): n is a, c, g, or t
tcctctgaaggaaggaaatgaggcaagcttggcagtttatgrtagaagagagaggagggtttctgtgttatgaactgcaygagttggntgagctcagtctatcacgtgtgtggtgggcacatggccagactccatgtgccctggacttgagtgtggtcttcagtgtgggggcatggggctgagccag

SEQ ID NO: 265 - DNA, 26 bp, Artificial Sequence
  Primer rs16891982-r2-1_299_F-sp
gaatctggtattttaaacagtaggaa

SEQ ID NO: 266 - DNA, 22 bp, Artificial Sequence
  Primer rs16891982-r2-1_299_R-sp
atccctatagtgcacacaactc

SEQ ID NO: 267 - DNA, 217 bp, Artificial Sequence
  Amplicon rs16891982-r2-1_299
tccaagttgtgctagaccagaaacttttagaagacatccttaggagagagaaagacttacaagaataaagtgaggaaaacacggagttgatgcasaagccccaacatccaacctcgactcctctttcgtagatgrgaaactctgtggagttgtgtgcactatagggatccccgcggtacacaatctgaargagagattggaggctgttgaggtacaa

SEQ ID NO: 268 - DNA, 23 bp, Artificial Sequence
  Primer rs16891982_1666_F-sp
tccaagttgtgctagaccagaaa

SEQ ID NO: 269 - DNA, 23 bp, Artificial Sequence
  Primer rs16891982_1666_R-sp
ttgtacctcaacagcctccaatc

SEQ ID NO: 270 - DNA, 214 bp, Artificial Sequence
  Amplicon rs16891982_1666
gaatctggtattttaaacagtaggaaatacacatagaaatatcaaatccaagttgtgctagaccagaaacttttagaagacatccttaggagagagaaagacttacaagaataaagtgaggaaaacacggagttgatgcasaagccccaacatccaacctcgactcctctttcgtagatgrgaaactctgtggagttgtgtgcactatagggat

SEQ ID NO: 271 - DNA, 21 bp, Artificial Sequence
  Primer rs174547_t1_1524_F-sp
ccaggtaaagctggctgagga

SEQ ID NO: 272 - DNA, 23 bp, Artificial Sequence
  Primer rs174547_t1_1524_R-sp
atgcacattgatcatgaccggaa

SEQ ID NO: 273 - DNA, 227 bp, Artificial Sequence
  Amplicon rs174547_t1_1524
tgtcttggtcactcccttcacatggttgcagaacaagagcctcaggctaatgagaaaatgctgtttgggggactttttgtttttgctgttttcacctacgcayccttttcaatagttgtgttatgctccagtcttctgagtgactgtcccttacctgggtggaaacccagtccatgttccggtcatgatcaatgtgcatgggaatatggttcatctgtgtcacccac

SEQ ID NO: 274 - DNA, 23 bp, Artificial Sequence
  Primer rs174547_1119_F-sp
tgtcttggtcactcccttcacat

SEQ ID NO: 275 - DNA, 23 bp, Artificial Sequence
  Primer rs174547_1119_R-sp
gtgggtgacacagatgaaccata

SEQ ID NO: 276 - DNA, 233 bp, Artificial Sequence
  Amplicon rs174547_1119
ccaggtaaagctggctgaggaagggacatgagactgtcttggtcactcccttcacatggttgcagaacaagagcctcaggctaatgagaaaatgctgtttgggggactttttgtttttgctgttttcacctacgcayccttttcaatagttgtgttatgctccagtcttctgagtgactgtcccttacctgggtggaaacccagtccatgttccggtcatgatcaatgtgcat

SEQ ID NO: 277 - DNA, 16 bp, Artificial Sequence
  Primer rs17553719_1297_F-sp
ccagcgcctgtcggag

SEQ ID NO: 278 - DNA, 22 bp, Artificial Sequence
  Primer rs17553719_1297_R-sp
gtgtctccagcgctcctataaa

SEQ ID NO: 279 - DNA, 210 bp, Artificial Sequence
  Amplicon rs17553719_1297
ggtagcggatgtggagcatctccccgcagcgggacaccagctccttctgtcgacccatggcggggcaggcggcggckctgtcgggcgggcaggggtggcgggaggcggyggcgcagcgagcagcggcctccagcgctggtggctccctttataggagcgctggagacacgggccccgcccgccctgcagccccgccctgcagtcccggag

SEQ ID NO: 280 - DNA, 16 bp, Artificial Sequence
  Primer rs17553719-r2-1_3_F-sp
gtcggagcagccggta

SEQ ID NO: 281 - DNA, 20 bp, Artificial Sequence
  Primer rs17553719-r2-1_3_R-sp
cctataaagggagccaccag

SEQ ID NO: 282 - DNA, 190 bp, Artificial Sequence
  Amplicon rs17553719-r2-1_3
ccagcgcctgtcggagcagccggtagcggatgtggagcatctccccgcagcgggacaccagctccttctgtcgacccatggcggggcaggcggcggckctgtcgggcgggcaggggtggcgggaggcggyggcgcagcgagcagcggcctccagcgctggtggctccctttataggagcgctggagacac

SEQ ID NO: 283 - DNA, 21 bp, Artificial Sequence
  Primer rs17553719_1110_F-sp
ggtagcggatgtggagcatct

SEQ ID NO: 284 - DNA, 16 bp, Artificial Sequence
  Primer rs17553719_1110_R-sp
ctccgggactgcaggg

SEQ ID NO: 285 - DNA, 167 bp, Artificial Sequence
  Amplicon rs17553719_1110
gtcggagcagccggtagcggatgtggagcatctccccgcagcgggacaccagctccttctgtcgacccatggcggggcaggcggcggckctgtcgggcgggcaggggtggcgggaggcggyggcgcagcgagcagcggcctccagcgctggtggctccctttatagg

SEQ ID NO: 286 - DNA, 22 bp, Artificial Sequence
  Primer rs17728338-r1-1_606_F-sp
ccactcattaagtgtaaactgc

SEQ ID NO: 287 - DNA, 20 bp, Artificial Sequence
  Primer rs17728338-r1-1_606_R-sp
gttcttgagtggggaaggtg

SEQ ID NO: 288 - DNA, 180 bp, Artificial Sequence
  Amplicon rs17728338-r1-1_606
ggctactctcctcctctgctaaaactccaagaaaaaaatgtggtttgttcagcyggcaccgagctcttagtaggaccrttgcaaaaggctcccgaatggcaccctgccttcctacatttgtcctyagagcatagctagacgcacaccttccccactcaagaactccaaaggcttccccta

SEQ ID NO: 289 - DNA, 22 bp, Artificial Sequence
  Primer rs17728338_1649_F-sp
ggctactctcctcctctgctaa

SEQ ID NO: 290 - DNA, 23 bp, Artificial Sequence
  Primer rs17728338_1649_R-sp
taggggaagcctttggagttctt

SEQ ID NO: 291 - DNA, 222 bp, Artificial Sequence
  Amplicon rs17728338_1649
ccactcattaagtgtaaactgcatgcatgcatgaatggggtrctctggttcagaacagtggctactctcctcctctgctaaaactccaagaaaaaaatgtggtttgttcagcyggcaccgagctcttagtaggaccrttgcaaaaggctcccgaatggcaccctgccttcctacatttgtcctyagagcatagctagacgcacaccttccccactcaagaac

SEQ ID NO: 292 - DNA, 20 bp, Artificial Sequence
  Primer rs1799750_3_F-sp
cccacctttcccactgtatc

SEQ ID NO: 293 - DNA, 22 bp, Artificial Sequence
  Primer rs1799750_3_R-sp
tgccacttagatgaggaaattg

SEQ ID NO: 294 - DNA, 229 bp, Artificial Sequence
  Amplicon rs1799750_3
  misc_feature (111)..(111): n is a, c, g, or t
  misc_feature (113)..(113): n is a, c, g, or t
caacactttcctccccttatggattcctgttttctttctgcgtcaagactgatatcttactcataaacaatacttcagtatatcttggattgatttgagataagtcatatncnttctaattatttaactacaatttcctcatctaagtggcataacatgtgagttcaagagggtaagaagaatgggaagacattctcacaaaaaggaaatcctcttctcagaggcatca

SEQ ID NO: 295 - DNA, 23 bp, Artificial Sequence
  Primer rs1799750_1779_F-sp
ttgcttttcaacactttcctccc

SEQ ID NO: 296 - DNA, 26 bp, Artificial Sequence
  Primer rs1799750_1779_R-sp
cctttttgtgagaatgtcttcccatt

SEQ ID NO: 297 - DNA, 214 bp, Artificial Sequence
  Amplicon rs1799750_1779
  misc_feature (119)..(119): n is a, c, g, or t
  misc_feature (121)..(121): n is a, c, g, or t
ttgcttttcaacactttcctccccttatggattcctgttttctttctgcgtcaagactgatatcttactcataaacaatacttcagtatatcttggattgatttgagataagtcatatncnttctaattatttaactacaatttcctcatctaagtggcataacatgtgagttcaagagggtaagaagaatgggaagacattctcacaaaaagg

SEQ ID NO: 298 - DNA, 23 bp, Artificial Sequence
  Primer rs1799750_1110_F-sp
caacactttcctccccttatgga

SEQ ID NO: 299 - DNA, 23 bp, Artificial Sequence
  Primer rs1799750_1110_R-sp
tgatgcctctgagaagaggattt

SEQ ID NO: 300 - DNA, 184 bp, Artificial Sequence
  Amplicon rs1799750_1110
cccacctttcccactgtatcaggtttgcttttcaacactttcctccccttatggattcctgttttctttctgcgtcaagactgatatcttactcataaacaatacttcagtatatcttggattgatttgagataagtcatatcctttctaattatttaactacaatttcctcatctaagtggca

SEQ ID NO: 301 - DNA, 23 bp, Artificial Sequence
  Primer rs1799752-r2-1_1891_F-sp
aaaaggagaggagagagactcaa

SEQ ID NO: 302 - DNA, 22 bp, Artificial Sequence
  Primer rs1799752-r2-1_1891_R-sp
ttgtaaggggagctcagagaat

SEQ ID NO: 303 - DNA, 211 bp, Artificial Sequence
  Amplicon rs1799752-r2-1_1891
gcccctcacaggactgctgaggccctgcaggtgtctgcagcatgtggccccaggccggggactctgtaagccactgctggagagccactcccatcctttctcccatttctctagacctgctgcctatacagtcacttttatgtggtttcgccaattttattccagctctgaaattctctgagctccccttacaagcagaggtgagctaagg

SEQ ID NO: 304 - DNA, 17 bp, Artificial Sequence
  Primer rs1799752_1474_F-sp
gcccctcacaggactgc

SEQ ID NO: 305 - DNA, 23 bp, Artificial Sequence
  Primer rs1799752_1474_R-sp
ccttagctcacctctgcttgtaa

SEQ ID NO: 306 - DNA, 221 bp, Artificial Sequence
  Amplicon rs1799752_1474
aaaaggagaggagagagactcaagcaygcccctcacaggactgctgaggccctgcaggtgtctgcagcatgtggccccaggccggggactctgtaagccactgctggagagccactcccatcctttctcccatttctctagacctgctgcctatacagtcacttttatgtggtttcgccaattttattccagctctgaaattctctgagctccccttacaa

SEQ ID NO: 307 - DNA, 23 bp, Artificial Sequence
  Primer rs1800566_1874_F-sp
aagaggctgcttggagcaaaata

SEQ ID NO: 308 - DNA, 23 bp, Artificial Sequence
  Primer rs1800566_1874_R-sp
tgcatggaattggttgacttacc

SEQ ID NO: 309 - DNA, 214 bp, Artificial Sequence
  Amplicon rs1800566_1874
attctccaggcgtttcttccatccttccaggatttgaattcgggcgtctgctggagtgtgcccaatgctatatgtcagttgagrttctaagacttggaagccacagaaatgcagaatgccactctgaggatacagaaagcacagagaggtaagtcaaccaattccatgcagttgtactataaacaacagaagttggtctgggcttctcagtaag

SEQ ID NO: 310 - DNA, 23 bp, Artificial Sequence
  Primer rs1800566_1408_F-sp
attctccaggcgtttcttccatc

SEQ ID NO: 311 - DNA, 23 bp, Artificial Sequence
  Primer rs1800566_1408_R-sp
cttactgagaagcccagaccaac

SEQ ID NO: 312 - DNA, 214 bp, Artificial Sequence
  Amplicon rs1800566_1408
aagaggctgcttggagcaaaatacagtggtgtctcatcccaaatattctccaggcgtttcttccatccttccaggatttgaattcgggcgtctgctggagtgtgcccaatgctatatgtcagttgagrttctaagacttggaagccacagaaatgcagaatgccactctgaggatacagaaagcacagagaggtaagtcaaccaattccatgca

SEQ ID NO: 313 - DNA, 23 bp, Artificial Sequence
  Primer rs1800624_1137_F-sp
gagcccccgatcctatttattcc

SEQ ID NO: 314 - DNA, 26 bp, Artificial Sequence
  Primer rs1800624_1137_R-sp
tcaaaaaacatgagaaaccccagaaa

SEQ ID NO: 315 - DNA, 230 bp, Artificial Sequence
  Amplicon rs1800624_1137
gagcccccgatcctatttattccatcagtccatcagggctgcctggtgacccactggagccccatcttgattgcgcaaagttgcatcaatagggttcaggccagactgttgtctgcaagggtgcawttgggcctgcatcatgaaggcaaggctggggaacaggagagaaacctgtttggarcttcgtgaaagaaaatcattttttttctggggtttctcatgttttttga

SEQ ID NO: 316 - DNA, 18 bp, Artificial Sequence
  Primer rs1800625-r1-1_1386_F-sp
gagccccatcttgattgc

SEQ ID NO: 317 - DNA, 23 bp, Artificial Sequence
  Primer rs1800625-r1-1_1386_R-sp
aaatttcttttttccctgggttt

SEQ ID NO: 318 - DNA, 212 bp, Artificial Sequence
  Amplicon rs1800625-r1-1_1386
gagccccatcttgattgcgcaaagttgcatcaatagggttcaggccagactgttgtctgcaagggtgcawttgggcctgcatcatgaaggcaaggctggggaacaggagagaaacctgtttggarcttcgtgaaagaaaatcattttttttctggggtttctcatgttttttgaaaaaaattctcaactaaacccagggaaaaaagaaattt

SEQ ID NO: 319 - DNA, 17 bp, Artificial Sequence
  Primer rs1801131_t1_124_F-sp
ctggggtcaggccaggg

SEQ ID NO: 320 - DNA, 22 bp, Artificial Sequence
  Primer rs1801131_t1_124_R-sp
ctttggggagctgaaggactac

SEQ ID NO: 321 - DNA, 212 bp, Artificial Sequence
  Amplicon rs1801131_t1_124
gatgaaccagggtccccactccagcatcactcactttgtgaccattccggtttggttctcccgagaggtaaagaacraagacttcaaagacacktkcttcactggtcagctcctccccccacatcttcagcagctcctccttgggggacttgctcttcaggtagaagaggtagtagtccttcagctccccaaaggcaggggaagaggaattg

SEQ ID NO: 322 - DNA, 21 bp, Artificial Sequence
  Primer rs1801131_1179_F-sp
gatgaaccagggtccccactc

SEQ ID NO: 323 - DNA, 22 bp, Artificial Sequence
  Primer rs1801131_1179_R-sp
caattcctcttcccctgccttt

SEQ ID NO: 324 - DNA, 217 bp, Artificial Sequence
  Amplicon rs1801131_1179
ctggggtcaggccaggggcaggggatgaaccagggtccccactccagcatcactcactttgtgaccattccggtttggttctcccgagaggtaaagaacraagacttcaaagacacktkcttcactggtcagctcctccccccacatcttcagcagctcctccttgggggacttgctcttcaggtagaagaggtagtagtccttcagctccccaaag

SEQ ID NO: 325 - DNA, 22 bp, Artificial Sequence
  Primer rs1801133_194_F-sp
ctcacctggatgggaaagatcc

SEQ ID NO: 326 - DNA, 23 bp, Artificial Sequence
  Primer rs1801133_194_R-sp
cctctcctgactgtcatccctat

SEQ ID NO: 327 - DNA, 228 bp, Artificial Sequence
  Amplicon rs1801133_194
ctcacctggatgggaaagatcccggggacgatggggcaagtgatgcccatgtcggtgcatgccttcacaaagcggaagaatgtgtcagcctcaaagaaaagctgcgtgatgatgaaatcgrctcccgcagacaccttctccttcaagtgcttcaggtcagcctcaaagctccctgcttcggggtggcctttggggtaacctgccaatagggatgacagtcaggagagg

SEQ ID NO: 328 - DNA, 20 bp, Artificial Sequence
  Primer rs1801133_t1_1238_F-sp
caagtgatgcccatgtcggt

SEQ ID NO: 329 - DNA, 19 bp, Artificial Sequence
  Primer rs1801133_t1_1238_R-sp
tccctcgccttgaacaggt

SEQ ID NO: 330 - DNA, 220 bp, Artificial Sequence
  Amplicon rs1801133_t1_1238
caagtgatgcccatgtcggtgcatgccttcacaaagcggaagaatgtgtcagcctcaaagaaaagctgcgtgatgatgaaatcgrctcccgcagacaccttctccttcaagtgcttcaggtcagcctcaaagctccctgcttcggggtggcctttggggtaacctgccaatagggatgacagtcaggagaggctggcctccacctgttcaaggcgaggga

SEQ ID NO: 331 - DNA, 22 bp, Artificial Sequence
  Primer rs1805005_2_F-sp
ctggaggtgtccatctctgacg

SEQ ID NO: 332 - DNA, 18 bp, Artificial Sequence
  Primer rs1805005_2_R-sp
gttgctcccgctcaccag

SEQ ID NO: 333 - DNA, 189 bp, Artificial Sequence
  Amplicon rs1805005_2
cacccccacagccatcccccagctggggctggctgccaaccagacaggagcccggtgcctggaggtgtccatctctgacgggctcytcctcagcctggggctggtgagcttggtggagaacgcgctggtgktggccaccatcgccaagaaccggaacctgcactcacccatgtactgcttcatctgctg

SEQ ID NO: 334 - DNA, 17 bp, Artificial Sequence
  Primer rs1805005_1_F-sp
cacccccacagccatcc

SEQ ID NO: 335 - DNA, 23 bp, Artificial Sequence
  Primer rs1805005_1_R-sp
cagcagatgaagcagtacatggg

SEQ ID NO: 336 - DNA, 168 bp, Artificial Sequence
  Amplicon rs1805005_1
ctggaggtgtccatctctgacgggctcytcctcagcctggggctggtgagcttggtggagaacgcgctggtgktggccaccatcgccaagaaccggaacctgcactcacccatgtactgcttcatctgctgcctggccttgtcggamctgctggtgagcgggagcaac

SEQ ID NO: 337 - DNA, 18 bp, Artificial Sequence
  Primer rs1805006-r2-1_1161_F-sp
tccatctctgacgggctc

SEQ ID NO: 338 - DNA, 17 bp, Artificial Sequence
  Primer rs1805006-r2-1_1161_R-sp
ccagcaggaggatgacg

SEQ ID NO: 339 - DNA, 190 bp, Artificial Sequence
  Amplicon rs1805006-r2-1_1161
tgcactcacccatgtactgcttcatctgctgcctggccttgtcggacctgctggtgagcgggagcaacgtgctggagacggccgtcatcctcctgctggaggccggtgcactggtggcccgggctgcggtgctgcagcagctggacaatgtcattgacgtgatcacctgcagctccatgctgtccagcct

SEQ ID NO: 340 - DNA, 20 bp, Artificial Sequence
  Primer rs1805006_2_F-sp
tgcactcacccatgtactgc

SEQ ID NO: 341 - DNA, 18 bp, Artificial Sequence
  Primer rs1805006_2_R-sp
aggctggacagcatggag

SEQ ID NO: 342 - DNA, 190 bp, Artificial Sequence
  Amplicon rs1805006_2
tccatctctgacgggctcytcctcagcctggggctggtgagcttggtggagaacgcgctggtgktggccaccatcgccaagaaccggaacctgcactcacccatgtactgcttcatctgctgcctggccttgtcggamctgctggtgagcgggagcaacrtgctggagacggccgtcatcctcctgctgg

SEQ ID NO: 343 - DNA, 18 bp, Artificial Sequence
  Primer rs1805007_1322_F-sp
ctgcagcagctggacaat

SEQ ID NO: 344 - DNA, 23 bp, Artificial Sequence
  Primer rs1805007_1322_R-sp
tggtcgtagtaggcgatgaagag

SEQ ID NO: 345 - DNA, 170 bp, Artificial Sequence
  Amplicon rs1805007_1322
ctgtccagcctctgcttcctgggcgccatcgccgtggaccgctacatctccatcttctacgcactgcgctaccacagcatcgtgaccctgccgcgggcgcggcgagccgttgcggccatctgggtggccagtgtcgtcttcagcacgctcttcatcgcctactacgacca

SEQ ID NO: 346 - DNA, 20 bp, Artificial Sequence
  Primer rs11547464_rs1805007_F2R2_F-sp
ctgtccagcctctgcttcct

SEQ ID NO: 347 - DNA, 20 bp, Artificial Sequence
  Primer rs11547464_rs1805007_F2R2_R-sp
tggtcgtagtaggcgatgaa

SEQ ID NO: 348 - DNA, 218 bp, Artificial Sequence
  Amplicon rs11547464_rs1805007_F2R2
ctgcagcagctggacaatgtcaytgacgtgatcacctgcagctccatgctgtccagcctctgcttcctgggcgccatcgccgtggaccgctacatctccatcttctacgcactgbgctaccacagcatcgtgaycctgmcgygggcgcggcragccgttgcggccatytgggtgkccagtgtcgtcttcagcacgctcttcatcgcctactacgacca

SEQ ID NO: 349 - DNA, 20 bp, Artificial Sequence
  Primer rs1805008_3_F-sp
gtggaccgctacatctccat

SEQ ID NO: 350 - DNA, 20 bp, Artificial Sequence
  Primer rs1805008_3_R-sp
ccagcatagccaggaagaag

SEQ ID NO: 351 - DNA, 184 bp, Artificial Sequence
  Amplicon rs1805008_3
gtggaccgctacatctccatcttctacgcactgcgctaccacagcatcgtgaccctgccgcgggcgcggcgagccgttgcggccatctgggtggccagtgtcgtcttcagcacgctcttcatcgcctactacgaccacgtggccgtcctgctgtgcctcgtggtcttcttcctggctatgctgg

SEQ ID NO: 352 - DNA, 23 bp, Artificial Sequence
  rs1805009_1955_F-sp
cttcttcctgcatctcacactca

SEQ ID NO: 353 - DNA, 21 bp, Artificial Sequence
  rs1805009_1955_R-sp
ctgcccagcacacttaaagcc

SEQ ID NO: 354 - DNA, 169 bp, Artificial Sequence
  Amplicon rs1805009_1955
cttcttcctgcatctcacactcatcgtcctctgccccgagcaccccacgtgcggctgcatcttcaagaacttcaacctctttctcgccctcatcatctgcaatgccatcatcsaccccctcatctacgccttycacagccaggagctccgcaggacgctcaaggaggtg

SEQ ID NO: 355 - DNA, 24 bp, Artificial Sequence
  Primer rs1805009_2_F-sp
gctgcatcttcaagaacttcaacc

SEQ ID NO: 356 - DNA, 23 bp, Artificial Sequence
  Primer rs1805009_2_R-sp
cacacaatatcaccacctccctc

SEQ ID NO: 357 - DNA, 221 bp, Artificial Sequence
  Amplicon rs1805009_2
cttcttcctgcatctcacactcatcgtcctctgccccgagcaccccacgtgcggctgcatcttcaagaacttcaacctctttctcgccctcatcatctgcaatgccatcatcsaccccctcatctacgccttycacagccaggagctccgcaggacgctcaaggaggtgctgacrtgctcctggtgagcgcggtgcacrcggctttaagtgtgctgggcag

SEQ ID NO: 358 - DNA, 23 bp, Artificial Sequence
  Primer rs1805009_1_F-sp
cttcttcctgcatctcacactca

SEQ ID NO: 359 - DNA, 19 bp, Artificial Sequence
  Primer rs1805009_1_R-sp
cacctccttgagcgtcctg

SEQ ID NO: 360 - DNA, 190 bp, Artificial Sequence
  Amplicon rs1805009_1
gctgcatcttcaagaacttcaacctctttctcgccctcatcatctgcaatgccatcatcsaccccctcatctacgccttycacagccaggagctccgcaggacgctcaaggaggtgctgacrtgctcctggtgagcgcggtgcacrcggctttaagtgtgctgggcagagggaggtggtgatattgtgtg

SEQ ID NO: 361 - DNA, 17 bp, Artificial Sequence
  Primer rs200519781-r2-1_11_F-sp
ctgagtgcctggagctg

SEQ ID NO: 362 - DNA, 18 bp, Artificial Sequence
  Primer rs200519781-r2-1_11_R-sp
tcaggccatggacaggat

SEQ ID NO: 363 - DNA, 220 bp, Artificial Sequence
  Amplicon rs200519781-r2-1_11
ctcgtggtgggatccttgtcttygtccagtgctggtcctggtccgcccatgggcrgactcagactgttcatgagtgctcacctggtagatgaaagaccctgaacgtccagaccttccccctgaccrgtcacgtgcggactcttggtggctctgctgatggggcccatcctgtccatggcctgacactgactgtgtgtctgactcctctgaatgtccctca

SEQ ID NO: 364 - DNA, 22 bp, Artificial Sequence
  Primer rs200519781_16_F-sp
ctcgtggtgggatccttgtctt

SEQ ID NO: 365 - DNA, 23 bp, Artificial Sequence
  Primer rs200519781_16_R-sp
tgagggacattcagaggagtcag

SEQ ID NO: 366 - DNA, 211 bp, Artificial Sequence
  Amplicon rs200519781_16
ctgagtgcctggagctgtctcgtgcctgctcgtggtgggatccttgtcttygtccagtgctggtcctggtccgcccatgggcrgactcagactgttcatgagtgctcacctggtagatgaaagaccctgaacgtccagaccttccccctgaccrgtcacgtgcggactcttggtggctctgctgatggggcccatcctgtccatggcctga

SEQ ID NO: 367 - DNA, 23 bp, Artificial Sequence
  Primer rs20541_1514_F-sp
ctctttgtcctgcagcagttttc

SEQ ID NO: 368 - DNA, 23 bp, Artificial Sequence
  Primer rs20541_1514_R-sp
cgcctacccaagacatttttgac

SEQ ID NO: 369 - DNA, 219 bp, Artificial Sequence
  Amplicon rs20541_1514
ctctttgtcctgcagcagttttccagcttgcatgtccgagacaccaaaatcgaggtggcccagtttgtaaaggacctgctcttacatttaaagaaactttttcgcgagggacrgttcaactgaaacttcgaaagcatcattatttgcagagacaggacctgactattgaagttgcagattcatttttctttctgatgtcaaaaatgtcttgggtaggcg

SEQ ID NO: 370 - DNA, 23 bp, Artificial Sequence
  Primer rs20541_t1_1386_F-sp
atgtccgagacaccaaaatcgag

SEQ ID NO: 371 - DNA, 23 bp, Artificial Sequence
  Primer rs20541_t1_1386_R-sp
gaattttacccctccctaaccct

SEQ ID NO: 372 - DNA, 217 bp, Artificial Sequence
  Amplicon rs20541_t1_1386
atgtccgagacaccaaaatcgaggtggcccagtttgtaaaggacctgctcttacatttaaagaaactttttcgcgagggacrgttcaactgaaacttcgaaagcatcattatttgcagagacaggacctgactattgaagttgcagattcatttttctttctgatgtcaaaaatgtcttgggtaggcgggaaggagggttagggaggggtaaaattc

SEQ ID NO: 373 - DNA, 24 bp, Artificial Sequence
  Primer rs2070600_t1_1192_F-sp
ggagaagaccctggaattcttacg

SEQ ID NO: 374 - DNA, 20 bp, Artificial Sequence
  Primer rs2070600_t1_1192_R-sp
tctagaacacaggccggaca

SEQ ID NO: 375 - DNA, 212 bp, Artificial Sequence
  Amplicon rs2070600_t1_1192
gactcggtagttggacttggtctcctttccattcctgttcattgcctggcaccggaaaatcccctcatcctggatcccsacagccggaaggaagagggagcygttgggaaggacacgagccacactgtcccaggggcctcctccctggggagacaggaccttccaagcttctgtccggcctgtgttctagaagcagagaagcagggcctaaa

SEQ ID NO: 376 - DNA, 23 bp, Artificial Sequence
  Primer rs2070600_111_F-sp
gactcggtagttggacttggtct

SEQ ID NO: 377 - DNA, 22 bp, Artificial Sequence
  Primer rs2070600_111_R-sp
tttaggccctgcttctctgctt

SEQ ID NO: 378 - DNA, 224 bp, Artificial Sequence
  Amplicon rs2070600_111
ggagaagaccctggaattcttacggtagacacggactcggtagttggacttggtctcctttccattcctgttcattgcctggcaccggaaaatcccctcatcctggatcccsacagccggaaggaagagggagcygttgggaaggacacgagccacactgtcccaggggcctcctccctggggagacaggaccttccaagcttctgtccggcctgtgttctaga

SEQ ID NO: 379 - DNA, 23 bp, Artificial Sequence
  Primer rs2082412_t1_1897_F-sp
cagaggtctgcacacataggaag

SEQ ID NO: 380 - DNA, 23 bp, Artificial Sequence
  Primer rs2082412_t1_1897_R-sp
ggcagcagaatttaaccacgaac

SEQ ID NO: 381 - DNA, 226 bp, Artificial Sequence
  Amplicon rs2082412_t1_1897
agatagatcaaaggcccccttccatttctcctctccagagtgttccagtaagaacatccccttctagctatttcacacatggacaaccaagaaatagtcatttacagarcattttgcatttgtacaatttcactcgttatttctcccccagtacctaatgggggctgcagcgtgtactctgttcgtggttaaattctgctgccagaagtttcctccagctcctgat

SEQ ID NO: 382 - DNA, 23 bp, Artificial Sequence
  Primer rs2082412_1242_F-sp
agatagatcaaaggcccccttcc

SEQ ID NO: 383 - DNA, 23 bp, Artificial Sequence
  Primer rs2082412_1242_R-sp
atcaggagctggaggaaacttct

SEQ ID NO: 384 - DNA, 224 bp, Artificial Sequence
  Amplicon rs2082412_1242
cagaggtctgcacacataggaagatagatcaaaggcccccttccatttctcctctccagagtgttccagtaagaacatccccttctagctatttcacacatggacaaccaagaaatagtcatttacagarcattttgcatttgtacaatttcactcgttatttctcccccagtacctaatgggggctgcagcgtgtactctgttcgtggttaaattctgctgcc

SEQ ID NO: 385 - DNA, 23 bp, Artificial Sequence
  Primer rs2201841_1108_F-sp
gactcaagaactctgcccacctt

SEQ ID NO: 386 - DNA, 25 bp, Artificial Sequence
  Primer rs2201841_1108_R-sp
tggttgtgattttggatttctcacc

SEQ ID NO: 387 - DNA, 217 bp, Artificial Sequence
  Amplicon rs2201841_1108
gactcaagaactctgcccaccttggcctcccaaagtgctgggcttacaggcaggagccaccatgcctggcctatgattatgctttttcttgargtcatcatcttctatattagtttcctattactactgtcacaaatcatcacaaacttgaaagcttaaaacaacatgaatttattatcttatagttctggaggtgagaaatccaaaatcacaacca

SEQ ID NO: 388 - DNA, 20 bp, Artificial Sequence
  Primer rs2201841_F1R1_F-sp
tgtttgccaagttggtcttg

SEQ ID NO: 389 - DNA, 20 bp, Artificial Sequence
  Primer rs2201841_F1R1_R-sp
gatttcagcccagtggttgt

SEQ ID NO: 390 - DNA, 258 bp, Artificial Sequence
  Amplicon rs2201841_F1R1
tgtttgccaagttggtcttgaactcctggactcaagaactctgcccaccttggcctcccaaagtgctgggcttacaggcaggagccaccatgcctggcctatgattatgctttttcttgaagtcatcatcttctatattagtttcctattactactgtcacaaatcatcacaaacttgaaagcttaaaacaacatgaatttattatcttatagttctggaggtgagaaatccaaaatcacaaccactgggctgaaatc

SEQ ID NO: 391 - DNA, 21 bp, Artificial Sequence
  Primer rs2201841_F2R2_F-sp
tcctggactcaagaactctgc

SEQ ID NO: 392 - DNA, 20 bp, Artificial Sequence
  Primer rs2201841_F2R2_R-sp
agcccagtggttgtgatttt

SEQ ID NO: 393 - DNA, 229 bp, Artificial Sequence
  Primer Amplicon rs2201841_F2R2
tcctggactcaagaactctgcccaccttggcctcccaaagtgctgggcttacaggcaggagccaccatgcctggcctatgattatgctttttcttgaagtcatcatcttctatattagtttcctattactactgtcacaaatcatcacaaacttgaaagcttaaaacaacatgaatttattatcttatagttctggaggtgagaaatccaaaatcacaaccactgggct

SEQ ID NO: 394 - DNA, 18 bp, Artificial Sequence
  Primer rs2228479-r2-1_1118_F-sp
atcgccaagaaccggaac

SEQ ID NO: 395 - DNA, 16 bp, Artificial Sequence
  Primer rs2228479-r2-1_1118_R-sp
gcgcccaggaagcaga

SEQ ID NO: 396 - DNA, 224 bp, Artificial Sequence
  Amplicon rs2228479-r2-1_1118
atcgccaagaaccggaacctgcactcacccatgtactgcttcatctgctgcctggccttgtcggamctgctggtgagcgggagcaacrtgctggagacggccgtcatcctcctgctggaggccggtgcactggtggcccgggctgcggtgctgcagcagctggacaatgtcaytgacgtgatcacctgcagctccatgctgtccagcctctgcttcctgggcgc

SEQ ID NO: 397 - DNA, 19 bp, Artificial Sequence
  Primer rs2282679-r2-1_1594_F-sp
gtgtgagcaactctctgtg

SEQ ID NO: 398 - DNA, 19 bp, Artificial Sequence
  Primer rs2282679-r2-1_1594_R-sp
ccagcaaatctctgtctct

SEQ ID NO: 399 - DNA, 230 bp, Artificial Sequence
  Amplicon rs2282679-r2-1_1594
tgctgggtgtgattctggtatctaaaatattttcacccaatctgattctacactgtgagccaattcatttatgtttttggcaacccagagggactactacttgcttccaaagctaacaataaaaaatacctggctttgtgagataattaagagacagagatttgctgggcatggtggctcacgcctgtaatcccagcactttgggaggccgaggtgggtggatcacaagg

SEQ ID NO: 400 - DNA, 24 bp, Artificial Sequence
  Primer rs2282679_M_REP_F-sp
aactctctgtgctgggtgtgattc

SEQ ID NO: 401 - DNA, 21 bp, Artificial Sequence
  Primer rs2282679_M_REP_R-sp
ccaccatgcccagcaaatctc

SEQ ID NO: 402 - DNA, 186 bp, Artificial Sequence
  Amplicon rs2282679_M_REP
aactctctgtgctgggtgtgattctggtatctaaaatattttcacccaatctgattctacactgtgagccaattcatttatgtttttggcaacccagagggactactacttgcttccaaagctaacaataaaaaatacctggctttgtgagataattaagagacagagatttgctgggcatggtgg

SEQ ID NO: 403 - DNA, 20 bp, Artificial Sequence
  Primer rs2282679_F1R1_F-sp
tgctgggtgtgattctggta

SEQ ID NO: 404 - DNA, 19 bp, Artificial Sequence
  Primer rs2282679_F1R1_R-sp
ccttgtgatccacccacct

SEQ ID NO: 405 - DNA, 185 bp, Artificial Sequence
  Amplicon rs2282679_F1R1
gtgtgagcaactctctgtgctgggtgtgattctggtatctaaaatattttcacccaatctgattctacactgtgagccaattcatttatgtttttggcaacccagagggactactacttgcttccaaagctaayaataaaaaatacctggctktgtgagataattaagagacagagatttgctgg

SEQ ID NO: 406 - DNA, 20 bp, Artificial Sequence
  Primer rs2555364_1652_F-sp
gctagcactgtgaccttggg

SEQ ID NO: 407 - DNA, 25 bp, Artificial Sequence
  Primer rs2555364_1652_R-sp
aggattctaacctctgtagtctgtt

SEQ ID NO: 408 - DNA, 214 bp, Artificial Sequence
  Amplicon rs2555364_1652
gctagcactgtgaccttgggcaaagttcctaacttccctctgcctctgttatctcagctgtgaaacagggtgattacaactttcagttcatagtgctgtgtgasaagtgcacgaagaaacacaagtgaactgcttacacaagtacctagcacatagaaaacattcactgaatgttcattgttacataagaacagactacagaggttagaatcct

SEQ ID NO: 409 - DNA, 23 bp, Artificial Sequence
  Primer rs2555364_t1_1994_F-sp
ggcaaagttcctaacttccctct

SEQ ID NO: 410 - DNA, 23 bp, Artificial Sequence
  Primer rs2555364_t1_1994_R-sp
ctccactttcatctttcctgagt

SEQ ID NO: 411 - DNA, 221 bp, Artificial Sequence
  Amplicon rs2555364_t1_1994
ggcaaagttcctaacttccctctgcctctgttatctcagctgtgaaacagggtgattacaactttcagttcatagtgctgtgtgasaagtgcacgaagaaacacaagtgaactgcttacacaagtacctagcacatagaaaacattcactgaatgttcattgttacataagaacagactacagaggttagaatcctttactcaggaaagatgaaagtggag

SEQ ID NO: 412 - DNA, 23 bp, Artificial Sequence
  Primer rs26722_t1_1498_F-sp
acaacaacaacaaagagcaagaa

SEQ ID NO: 413 - DNA, 22 bp, Artificial Sequence
  Primer rs26722_t1_1498_R-sp
ttacagaggttgcaaagggcat

SEQ ID NO: 414 - DNA, 213 bp, Artificial Sequence
  Amplicon rs26722_t1_1498
agaaaaaatgttgcatctttacctgttcagcatgatttttgttttttgctccctgcattgccagctctggatttacgtaaccatttttaactttctygatagaaccatactcgtacattccatctgatgacaatggagggtcctgaggggtttgctgtgggggaatgccctttgcaacctctgtaagtggggcttcagagatactgcacagat

SEQ ID NO: 415 - DNA, 26 bp, Artificial Sequence
  Primer rs26722_1351_F-sp
agaaaaaatgttgcatctttacctgt

SEQ ID NO: 416 - DNA, 23 bp, Artificial Sequence
  Primer rs26722_1351_R-sp
atctgtgcagtatctctgaagcc

SEQ ID NO: 417 - DNA, 224 bp, Artificial Sequence
  Amplicon rs26722_1351
acaacaacaacaaagagcaagaatattttcccttgtaaagaaaaaatgttgcatctttacctgttcagcatgatttttgttttttgctccctgcattgccagctctggatttacgtaaccatttttaactttctygatagaaccatactcgtacattccatctgatgacaatggagggtcctgaggggtttgctgtgggggaatgccctttgcaacctctgtaa

SEQ ID NO: 418 - DNA, 26 bp, Artificial Sequence
  Primer rs2917666_1439_F-sp
gtatgcatgttattcctcaacaaagt

SEQ ID NO: 419 - DNA, 26 bp, Artificial Sequence
  Primer rs2917666_1439_R-sp
tttttattctctcagtttttgccctt

SEQ ID NO: 420 - DNA, 195 bp, Artificial Sequence
  Amplicon rs2917666_1439
gcatgttattcctcaacaaagtccatttttaaaaagtagagtggttggagtgatgacgaacattatgaaagggcttattggcagagaaaaagtagaggggtmcagagactcaagttcctaasgaggctgaagtaagagtgggggattaaataagggcaaaaactgagagaataaaaaaagtgggttgtttggggc

SEQ ID NO: 421 - DNA, 25 bp, Artificial Sequence
  Primer rs2917666_1378_F-sp
gcatgttattcctcaacaaagtcca

SEQ ID NO: 422 - DNA, 21 bp, Artificial Sequence
  Primer rs2917666_1378_R-sp
gccccaaacaacccacttttt

SEQ ID NO: 423 - DNA, 181 bp, Artificial Sequence
  Amplicon rs2917666_1378
gtatgcatgttattcctcaacaaagtccatttttaaaaagtagagtggttggagtgatgacgaacattatgaaagggcttattggcagagaaaaagtagaggggtmcagagactcaagttcctaasgaggctgaagtaagagtgggggattaaataagggcaaaaactgagagaataaaaa

SEQ ID NO: 424 - DNA, 23 bp, Artificial Sequence
  Primer rs322458-r1-1_1671_F-sp
agatgcaacatggtgtaatagaa

SEQ ID NO: 425 - DNA, 23 bp, Artificial Sequence
  Primer rs322458-r1-1_1671_R-sp
cgtgttaggtgcatttaaagatt

SEQ ID NO: 426 - DNA, 213 bp, Artificial Sequence
  Amplicon rs322458-r1-1_1671
agatgcaacatggtgtaatagaatttgcctaggtgttggaatcagacttgactgcaaattctgattctgttactttttagctgtgtgaccttagataaattattaaacctttctgagcttyagttacctcttttttatctacaaaatggagataataagacataccttttactgtgaggattaataataaaatctttaaatgcacctaacacg

SEQ ID NO: 427 - DNA, 23 bp, Artificial Sequence
  Primer rs322458-r1-1_1791_F-sp
tagaatttgcctaggtgttggaa

SEQ ID NO: 428 - DNA, 23 bp, Artificial Sequence
  Primer rs322458-r1-1_1791_R-sp
tgaatacctaacgtgtacaatgc

SEQ ID NO: 429 - DNA, 220 bp, Artificial Sequence
  Amplicon rs322458-r1-1_1791
tagaatttgcctaggtgttggaatcagacttgactgcaaattctgattctgttactttttagctgtgtgaccttagataaattattaaacctttctgagcttyagttacctcttttttatctacaaaatggagataataagacataccttttactgtgaggattaataataaaatctttaaatgcacctaacacgatgcattgtacacgttaggtattca

SEQ ID NO: 430 - DNA, 20 bp, Artificial Sequence
  Primer rs33972313_t1_1989_F-sp
gcggatccagggtgcaatag

SEQ ID NO: 431 - DNA, 19 bp, Artificial Sequence
  Primer rs33972313_t1_1989_R-sp
cctccagtgccttcagtgc

SEQ ID NO: 432 - DNA, 180 bp, Artificial Sequence
  Amplicon rs33972313_t1_1989
catgatgtcaccacgggcatcggttcgtgcctggaagccataggcttttgggtctgtgggcagcacgtctgtcaaggtcaggacatagcagagcagccacayggtcatgatggccagcaygatctgaaggaggggggtgagggggcactgaaggcactggaggtctctgtccaggctaac

SEQ ID NO: 433 - DNA, 20 bp, Artificial Sequence
  Primer rs33972313_1103_F-sp
catgatgtcaccacgggcat

SEQ ID NO: 434 - DNA, 22 bp, Artificial Sequence
  Primer rs33972313_1103_R-sp
gttagcctggacagagacctcc

SEQ ID NO: 435 - DNA, 184 bp, Artificial Sequence
  Amplicon rs33972313_1103
gcggatccagggtgcaatagccatgatgtcaccacgggcatcggttcgtgcctggaagccataggcttttgggtctgtgggcagcacgtctgtcaaggtcaggacatagcagagcagccacayggtcatgatggccagcaygatctgaaggaggggggtgagggggcactgaaggcactggagg

SEQ ID NO: 436 - DNA, 25 bp, Artificial Sequence
  Primer rs35318931_151_F-sp
tgtaggaccatctcttcttttctca

SEQ ID NO: 437 - DNA, 20 bp, Artificial Sequence
  Primer rs35318931_151_R-sp
gttgatccaccaggaccctt

SEQ ID NO: 438 - DNA, 220 bp, Artificial Sequence
  Amplicon rs35318931_151
tgtaggaccatctcttcttttctcaagggaaaagtgtcaatcaggttgaacagggccacaggcatcaccagggagacatagcgctctttgtccatgccatgcttatccactagcaccatactgaagragtagagtgggattcgcagcaacagcctgtggcagacaaagaaaagaggagactaagtcaaaccatgactcccaagggtcctggtggatcaac

SEQ ID NO: 439 - DNA, 24 bp, Artificial Sequence
  Primer rs35318931_t1_127_F-sp
tcaagggaaaagtgtcaatcaggt

SEQ ID NO: 440 - DNA, 23 bp, Artificial Sequence
  Primer rs35318931_t1_127_R-sp
cttgggagtcatggtttgactta

SEQ ID NO: 441 - DNA, 181 bp, Artificial Sequence
  Amplicon rs35318931_t1_127
tcaagggaaaagtgtcaatcaggttgaacagggccacaggcatcaccagggagacatagcgctctttgtccatgccatgcttatccactagcaccatactgaagragtagagtgggattcgcagcaacagcctgtggcagacaaagaaaagaggagactaagtcaaaccatgactcccaag

SEQ ID NO: 442 - DNA, 23 bp, Artificial Sequence
  Primer rs374588791_1317_F-sp
catgaatggtgtcctgaccctct

SEQ ID NO: 443 - DNA, 17 bp, Artificial Sequence
  Primer rs374588791_1317_R-sp
gccccatcagcagagcc

SEQ ID NO: 444 - DNA, 221 bp, Artificial Sequence
  Amplicon rs374588791_1317
catgaatggtgtcctgaccctcttgggacgttgagtgcctggagctgtctcgtgcctgctygtggtgggatccttgtcttcctccagtgctggtcccggtccgtccatgggcggactcagactgttcatgagtgctcayctggtagaggaaagaccctgaacgtccagaccttcctgctgaccggccacgtgtggactsttggtggctctgctgatggggc

SEQ ID NO: 445 - DNA, 23 bp, Artificial Sequence
  Primer rs397507563_1173_F-sp
tgtctctcactgtcactgtcctg

SEQ ID NO: 446 - DNA, 23 bp, Artificial Sequence
  Primer rs397507563_1173_R-sp
tgcaagcagacaaactcgtaagg

SEQ ID NO: 447 - DNA, 192 bp, Artificial Sequence
  Amplicon rs397507563_1173
tgtctctcactgtcactgtcctggctaacactggatccctggygcctgcttgtcctggaccccgatgattgttcctgtcccacctgtgagtgtctagagctgtcagcccaagaggcagcttcatggtgacgtgaccctgagtgcctggagccgtctcctgattgtttgtccttacgagtttgtctgcttgca

SEQ ID NO: 448 - DNA, 23 bp, Artificial Sequence
  Primer rs397507563_1249_F-sp
gatgattgttcctgtcccacctg

SEQ ID NO: 449 - DNA, 20 bp, Artificial Sequence
  Primer rs397507563_1249_R-sp
cacatcccagggaaggtctg

SEQ ID NO: 450 - DNA, 180 bp, Artificial Sequence
  Amplicon rs397507563_1249
gatgattgttcctgtcccacctgtgagtgtctagagctgtcagcccaagaggcagcttcatggtgacgtgaccctgagtgcctggagccgtctcctgattgtttgtccttacgagtttgtctgcttgcacttctggatcctgactgcccatggkaggcatcagaccttccctgggatgtg

SEQ ID NO: 451 - DNA, 21 bp, Artificial Sequence
  Primer rs429358_t1_1648_F-sp
gaccatgaaggagttgaaggc

SEQ ID NO: 452 - DNA, 16 bp, Artificial Sequence
  Primer rs429358_t1_1648_R-sp
agcttgcgcaggtggg

SEQ ID NO: 453 - DNA, 210 bp, Artificial Sequence
  Amplicon rs429358_t1_1648
acaaatcggaactggaggaacaactgaccccggtggcggaggagacgcgggcacggctgtccaaggagctgcaggcggcgcaggcccggctgggcgcggacatggaggacgtgygcggccgcctggtgcagtaccgcggcgaggtgcaggccatgctcggccagagcaccgaggagctgcrggtgmgcctcgcctcccacctgcgcaagc

SEQ ID NO: 454 - DNA, 23 bp, Artificial Sequence
  Primer rs429358_143_F-sp
acaaatcggaactggaggaacaa

SEQ ID NO: 455 - DNA, 18 bp, Artificial Sequence
  Primer rs429358_143_R-sp
atcggcatcgcggaggag

SEQ ID NO: 456 - DNA, 239 bp, Artificial Sequence
  Amplicon rs429358_143
acaaatcggaactggaggaacaactgaccccggtggcggaggagacgcgggcacggctgtccaaggagctgcaggcggcgcaggcccggctgggcgcggacatggaggacgtgygcggccgcctggtgcagtaccgcggcgaggtgcaggccatgctcggccagagcaccgaggagctgcrggtgmgcctcgcctcccacctgcgcaagctgyvtvagcggctcctccgcgatgccgat

SEQ ID NO: 457 - DNA, 18 bp, Artificial Sequence
  Primer rs429358_2_F-sp
cacggctgtccaaggagc

SEQ ID NO: 458 - DNA, 17 bp, Artificial Sequence
  Primer rs429358_2_R-sp
tcatcggcatcgcggag

SEQ ID NO: 459 - DNA, 190 bp, Artificial Sequence
  Amplicon rs429358_2
cacggctgtccaaggagctgcaggcggcgcaggcccggctgggcgcggacatggaggacgtgygcggccgcctggtgcagtaccgcggcgaggtgcaggccatgctcggccagagcaccgaggagctgcrggtgmgcctcgcctcccacctgcgcaagctgyvtvagcggctcctccgcgatgccgatga

SEQ ID NO: 460 - DNA, 23 bp, Artificial Sequence
  Primer rs429358_1_F-sp
acaaatcggaactggaggaacaa

SEQ ID NO: 461 - DNA, 17 bp, Artificial Sequence
  Primer rs429358_1_R-sp
gcttgcgcaggtgggag

SEQ ID NO: 462 - DNA, 234 bp, Artificial Sequence
  Amplicon rs429358_1
gaccatgaaggagttgaaggcctacaaatcggaactggaggaacaactgaccccggtggcggaggagacgcgggcacggctgtccaaggagctgcaggcggcgcaggcccggctgggcgcggacatggaggacgtgygcggccgcctggtgcagtaccgcggcgaggtgcaggccatgctcggccagagcaccgaggagctgcrggtgmgcctcgcctcccacctgcgcaagct

SEQ ID NO: 463 - DNA, 22 bp, Artificial Sequence
  Primer rs4340_12_F-sp
ggagaggagagagactcaagca

SEQ ID NO: 464 - DNA, 20 bp, Artificial Sequence
  Primer rs4340_12_R-sp
ggggagctcagagaatttca

SEQ ID NO: 465 - DNA, 211 bp, Artificial Sequence
  Amplicon rs4340_12
ggagaggagagagactcaagcacgcccctcacaggactgctgaggccctgcaggtgtctgcagcatgtggccccaggccggggactctgtaagccactgctggagagccactcccatcctttctcccatttctctagacctgctgcctatacagtcacttttatgtggtttcgccaattttattccagctctgaaattctctgagctcccc

SEQ ID NO: 466 - DNA, 23 bp, Artificial Sequence
  Primer rs4340_6_F-sp
ctcccatttctctagacctgctg

SEQ ID NO: 467 - DNA, 22 bp, Artificial Sequence
  Primer rs4340_6_R-sp
gtggccatcacattcgtcagat

SEQ ID NO: 468 - DNA, 166 bp, Artificial Sequence
  Amplicon rs4340_6
ctcccatttctctagacctgctgcctatacagtcacttttatgtggtttcgccaattttattccagctctgaaattctctgagctccccttacaagcagaggtgagctaagggctggagctcaagscattcaamcccctaccagatctgacgaatgtgatggccac

SEQ ID NO: 469 - DNA, 23 bp, Artificial Sequence
  Primer rs4654748_1406_F-sp
acgttttccaagaacacacagga

SEQ ID NO: 470 - DNA, 23 bp, Artificial Sequence
  Primer rs4654748_1406_R-sp
ctcctttgttccttcccctcaac

SEQ ID NO: 471 - DNA, 198 bp, Artificial Sequence
  Amplicon rs4654748_1406
acgttttccaagaacacacaggaggkctatgggtagaggaaggtgtctgtccatctatccctggcccccagcccatgtggttttggcagcaatagggrtgtggggtaatgtcycccaaaattaaaatggtatatgtgtgtatgagaaggaaaggggggcaaagctgtggggaacggttgaggggaaggaacaaaggag

SEQ ID NO: 472 - DNA, 23 bp, Artificial Sequence
  Primer rs4654748_t1_1182_F-sp
agaggaaggtgtctgtccatcta

SEQ ID NO: 473 - DNA, 25 bp, Artificial Sequence
  Primer rs4654748_t1_1182_R-sp
caagaaatgttatgaaatggcctcc

SEQ ID NO: 474 - DNA, 214 bp, Artificial Sequence
  Amplicon rs4654748_t1_1182
agaggaaggtgtctgtccatctatccctggcccccagcccatgtggttttggcagcaatagggrtgtggggtaatgtcycccaaaattaaaatggtatatgtgtgtatgagaaggaaaggggggcaaagctgtggggaacggttgaggggaaggaacaaaggaggtcagtactgggaacgctgaagrtgggaggccatttcataacatttcttg

SEQ ID NO: 475 - DNA, 27 bp, Artificial Sequence
  Primer rs4746_F1R1_F-sp
tgtggaaaataaaaataatcatatctc

SEQ ID NO: 476 - DNA, 21 bp, Artificial Sequence
  Primer rs4746_F1R1_R-sp
cgtggtaagatttcaaagatg

SEQ ID NO: 477 - DNA, 190 bp, Artificial Sequence
  Amplicon rs4746_F1R1
taaaaacaggcaaacttaccgaawcctcgagggtctgaattgccattgtggtaactctgggtckcatcatcttcagtgccccaattgctacaaaaggaaggaaaacatagctacaatgtcctagggaatagatattctgaaaagcaagtaacatctttgaaatcttaccacgtttatatgctactttaca

SEQ ID NO: 478 - DNA, 25 bp, Artificial Sequence
  Primer rs4746-r2-1_3_F-sp
acatatatcacataaaaacaggcaa

SEQ ID NO: 479 - DNA, 23 bp, Artificial Sequence
  Primer rs4746-r2-1_3_R-sp
aggggttaggccaattatgtaaa

SEQ ID NO: 480 - DNA, 300 bp, Artificial Sequence
  Amplicon rs4746-r2-1_3
tgtggaaaataaaaataatcatatctctaataaattgatcatacttaattaggacattaatgttttaggtttattaaaatcataattatatacataatacaaaatcacacaaatctacatatatcacataaaaacaggcaaacttaccgaatcctcgagggtctgaattgccattgtggtaactctgggtctcatcatcttcagtgccccaattgctacaaaaggaaggaaaacatagctacaatgtcctagggaatagatattctgaaaagcaagtaacatctttgaaatcttaccacg

SEQ ID NO: 481 - DNA, 23 bp, Artificial Sequence
  Primer rs4746_1_F-sp
taaaaacaggcaaacttaccgaa

SEQ ID NO: 482 - DNA, 24 bp, Artificial Sequence
  Primer rs4746_1_R-sp
tgtaaagtagcatataaacgtggt

SEQ ID NO: 483 - DNA, 219 bp, Artificial Sequence
  Amplicon rs4746_1
acatatatcacataaaaacaggcaaacttaccgaawcctcgagggtctgaattgccattgtggtaactctgggtckcatcatcttcagtgccccaattgctacaaaaggaaggaaaacatagctacaatgtcctagggaatagatattctgaaaagcaagtaacatctttgaaatcttaccacgtttatatgctactttacataattggcctaacccct

SEQ ID NO: 484 - DNA, 20 bp, Artificial Sequence
  Primer rs4880_3_F-sp
ggtgacgttcaggttgttca

SEQ ID NO: 485 - DNA, 20 bp, Artificial Sequence
  Primer rs4880_3_R-sp
ggctgtgctttctcgtcttc

SEQ ID NO: 486 - DNA, 222 bp, Artificial Sequence
  Amplicon rs4880_3
gtggtgcttgctgtggtgcagctgcatgatctgcgcgttgatgtgaggttccagggcgccgtagtcgtagggcaggtcggggaggctgtgcttctgcctggagcccagataccccaaarccggagccagctgcctgctggtgctgaagacgagaaagcacagcccggtcagtcagcgccgcgggaccgtctacgcaggctgggctgccgaggaacggcagcg

SEQ ID NO: 487 - DNA, 18 bp, Artificial Sequence
  Primer rs4880_1271_F-sp
gtggtgcttgctgtggtg

SEQ ID NO: 488 - DNA, 16 bp, Artificial Sequence
  Primer rs4880_1271_R-sp
cgctgccgttcctcgg

SEQ ID NO: 489 - DNA, 194 bp, Artificial Sequence
  Amplicon rs4880_1271
ggtgacgttcaggttgttcacgtaggccgcgtggtgcttgctgtggtgcagctgcatgatctgcgcgttgatgtgaggttccagggcgccgtagtcgtagggcaggtcggggaggctgtgcttctgcctggagcccagataccccaaaaccggagccagctgcctgctggtgctgaagacgagaaagcacagcc

SEQ ID NO: 490 - DNA, 22 bp, Artificial Sequence
  Primer rs4880_t1_1131_F-sp
catgatctgcgcgttgatgtga

SEQ ID NO: 491 - DNA, 17 bp, Artificial Sequence
  Primer rs4880_t1_1131_R-sp
agcccagcctgcgtaga

SEQ ID NO: 492 - DNA, 181 bp, Artificial Sequence
  Amplicon rs4880_t1_1131
catgatctgcgcgttgatgtgaggttccagggcgccgtagtcgtagggcaggtcggggaggctgtgcttctgcctggagcccagataccccaaarccggagccagctgcctgctggtgctgaagacgagaaagcacagcccggtcagtcagcgccgcgggaccgtctacgcaggctgggct

SEQ ID NO: 493 - DNA, 25 bp, Artificial Sequence
  Primer rs4911414_t1_1235_F-sp
agctatgattgcacaactgtattct

SEQ ID NO: 494 - DNA, 23 bp, Artificial Sequence
  Primer rs4911414_t1_1235_R-sp
accatcctcaaggtccatagtca

SEQ ID NO: 495 - DNA, 210 bp, Artificial Sequence
  Amplicon rs4911414_t1_1235
agcaagatcctgtctctacctgaaaaaaaaaaaaatcctaaaaatgggcccccagtctctttttgtttgtaagtctttgctgagaaattcattkttagtctaatatgatgcttttctctagttgcctttaagattttttctttcacattaaccctggatactctgatgactatggaccttgaggatggtcatcttgcatactatctcaca

SEQ ID NO: 496 - DNA, 22 bp, Artificial Sequence
  Primer rs4911414_1811_F-sp
agcaagatcctgtctctacctg

SEQ ID NO: 497 - DNA, 25 bp, Artificial Sequence
  Primer rs4911414_1811_R-sp
tgtgagatagtatgcaagatgacca

SEQ ID NO: 498 - DNA, 228 bp, Artificial Sequence
  Primer Amplicon rs4911414_1811
agctatgattgcacaactgtattctagcctgggcaacagagcaagatcctgtctctacctgaaaaaaaaaaaaatcctaaaaatgggcccccagtctctttttgtttgtaagtctttgctgagaaattcattkttagtctaatatgatgcttttctctagttgcctttaagattttttctttcacattaaccctggatactctgatgactatggaccttgaggatggt

SEQ ID NO: 499 - DNA, 22 bp, Artificial Sequence
  Primer rs4911442_t1_1476_F-sp
gaaatgtttgctacactgctca

SEQ ID NO: 500 - DNA, 24 bp, Artificial Sequence
  Primer rs4911442_t1_1476_R-sp
tgaactgatcatagaattggaact

SEQ ID NO: 501 - DNA, 186 bp, Artificial Sequence
  Amplicon rs4911442_t1_1476
gaaatgtttgctacactgctcagggctcttctaccttcaagcatgagctcaaaagatggtaggtattattatttcattttatatgtgaagaaagtattgcttggggaagtacagtaactagcttgaggtaacctgtaaatggtagtaccagaatraaaacctagttccaattctatgatcagttca

SEQ ID NO: 502 - DNA, 21 bp, Artificial Sequence
  Primer rs4911442-r2-1_1334_F-sp
ggctcttctaccttcaagcat

SEQ ID NO: 503 - DNA, 25 bp, Artificial Sequence
  Primer rs4911442-r2-1_1334_R-sp
tgctattctgaactgtagctaatga

SEQ ID NO: 504 - DNA, 185 bp, Artificial Sequence
  Amplicon rs4911442-r2-1_1334
ggctcttctaccttcaagcatgagctcaaaagatggtaggtattattatttcattttatatgtgaagaaagtattgcttggggaagtacagtaactagcttgaggtaacctgtaaatggtagtaccagaatraaaacctagttccaattctatgatcagttcattagctacagttcagaatagca

SEQ ID NO: 505 - DNA, 17 bp, Artificial Sequence
  Primer rs558269137-r2-1_48_F-sp
gggccctgtccatccat

SEQ ID NO: 506 - DNA, 19 bp, Artificial Sequence
  Primer rs558269137-r2-1_48_R-sp
gtcagagacagtggacacc

SEQ ID NO: 507 - DNA, 225 bp, Artificial Sequence
  Amplicon rs558269137-r2-1_48
ctgtttatgagtgctcacctggtagaggaaagaccctgaacgtcgaracctttcccctgaccggtcacgtgcggactcttggtggctctgctgatggtgaccagcctgtccatggcctgacackgactgtgtgtctgagtcttytgaatgtccctcactgtcagtggcctgactaccactgkaccctcggtgtccactgtctctgactgcagatgaagcttgtcc

SEQ ID NO: 508 - DNA, 21 bp, Artificial Sequence
  Primer rs558269137-r1-1_97_F-sp
ctgtttatgagtgctcacctg

SEQ ID NO: 509 - DNA, 21 bp, Artificial Sequence
  Primer rs558269137-r1-1_97_R-sp
ggacaagcttcatctgcagtc

SEQ ID NO: 510 - DNA, 237 bp, Artificial Sequence
  Amplicon rs558269137-r1-1_97
gggccctgtccatccatgggaggactcagactgtttatgagtgctcacctggtagaggaaagaccctgaacgtcgaracctttcccctgaccggtcacgtgcggactcttggtggctctgctgatggtgaccagcctgtccatggcctgacackgactgtgtgtctgagtcttytgaatgtccctcactgtcagtggcctgactaccactgkaccctcggtgtccactgtctctgac

SEQ ID NO: 511 - DNA, 19 bp, Artificial Sequence
  Primer rs602662_1829_F-sp
cgaccggcgatacctacag

SEQ ID NO: 512 - DNA, 23 bp, Artificial Sequence
  Primer rs602662_1829_R-sp
atggtcatgatggtgtggttaca

SEQ ID NO: 513 - DNA, 210 bp, Artificial Sequence
  Amplicon rs602662_1829
  misc_feature (158)..(158): n is a, c, g, or t
cgaccggcgatacctacagcaggccctggactggttccgagctcgctacagctccctcatcttcgtggtcaccagtaatggcatggcctggtgtcgggagaacattgacacctcccacrgtgatgtggtgtttgctggcgatggcattgagggctcanctgccaaagattttgctctactcacacagtgtaaccacaccatcatgaccat

SEQ ID NO: 514 - DNA, 23 bp, Artificial Sequence
  Primer rs602662_t1_1548_F-sp
ctcgctacagctccctcatcttc

SEQ ID NO: 515 - DNA, 19 bp, Artificial Sequence
  Primer rs602662_t1_1548_R-sp
gtctccgcccgtgaggtat

SEQ ID NO: 516 - DNA, 212 bp, Artificial Sequence
  Amplicon rs602662_t1_1548
  misc_feature (117)..(117): n is a, c, g, or t
ctcgctacagctccctcatcttcgtggtcaccagtaatggcatggcctggtgtcgggagaacattgacacctcccacrgtgatgtggtgtttgctggcgatggcattgagggctcanctgccaaagattttgctctactcacacagtgtaaccacaccatcatgaccattgggacgttcgggatctgggccgcatacctcacgggcggagac

SEQ ID NO: 517 - DNA, 25 bp, Artificial Sequence
  Primer rs610604_t1_1903_F-sp
atttttggatggaccaagattgatt

SEQ ID NO: 518 - DNA, 26 bp, Artificial Sequence
  Primer rs610604_t1_1903_R-sp
gttttagaatgtggattatggcaaga

SEQ ID NO: 519 - DNA, 210 bp, Artificial Sequence
  Amplicon rs610604_t1_1903
aaggaagtcttagcaaactaactgacatcattcttgactcgaacatgttgggaaatgtgtcagatcatgttgcgtgaaaagtgtkagctcttcatcacaggcctgcatttcagtgaatggttctacaattcttgccataatccacattctaaaactttgttctatgagctaatgatgtaaaatcttgtgtgtgattttgtgtattctcat

SEQ ID NO: 520 - DNA, 25 bp, Artificial Sequence
  Primer rs610604_1843_F-sp
aaggaagtcttagcaaactaactga

SEQ ID NO: 521 - DNA, 26 bp, Artificial Sequence
  Primer rs610604_1843_R-sp
atgagaatacacaaaatcacacacaa

SEQ ID NO: 522 - DNA, 204 bp, Artificial Sequence
  Primer Amplicon rs610604_1843
atttttggatggaccaagattgattaaatgaaactgtgattttaaaaataaggaagtcttagcaaactaactgacatcattcttgactcgaacatgttgggaaatgtgtcagatcatgttgcgtgaaaagtgtkagctcttcatcacaggcctgcatttcagtgaatggttctacaattcttgccataatccacattctaaaac

SEQ ID NO: 523 - DNA, 23 bp, Artificial Sequence
  Primer rs61816761_1345_F-sp
tgagtgtccagacctatttaccg

SEQ ID NO: 524 - DNA, 22 bp, Artificial Sequence
  Primer rs61816761_1345_R-sp
ttcagggtcttccctctaccag

SEQ ID NO: 525 - DNA, 235 bp, Artificial Sequence
  Amplicon rs61816761_1345
tgagtgtccagacctatttaccgrttgctcgtggtgggatccctgccttcctcctctgcttgaccccgggtktccacgaatggtgtcctgaccctcttgggacgctgaatgcctggagctgtctcrtgcctgctcgtggtgbgatccttgtcttcctccagtgctggtcccggtccgtccatgggcagagtcagrctgttcatgagtgctyacctggtagagggaagaccctgaa

SEQ ID NO: 526 - DNA, 20 bp, Artificial Sequence
  Primer rs61816761_t1_1692_F-sp
gccttcctcctctgcttgac

SEQ ID NO: 527 - DNA, 19 bp, Artificial Sequence
  Primer rs61816761_t1_1692_R-sp
gtcaggggaacggtctgga

SEQ ID NO: 528 - DNA, 212 bp, Artificial Sequence
  Amplicon rs61816761_t1_1692
gccttcctcctctgcttgaccccgggtktccacgaatggtgtcctgaccctcttgggacgctgaatgcctggagctgtctcrtgcctgctcgtggtgbgatccttgtcttcctccagtgctggtcccggtccgtccatgggcagagtcagrctgttcatgagtgctyacctggtagagggaagaccctgaacgtccagaccgttcccctgac

SEQ ID NO: 529 - DNA, 18 bp, Artificial Sequence
  Primer rs7412_2_F-sp
gcggacatggaggacgtg

SEQ ID NO: 530 - DNA, 16 bp, Artificial Sequence
  Primer rs7412_2_R-sp
gccccggcctggtaca

SEQ ID NO: 531 - DNA, 217 bp, Artificial Sequence
  Amplicon rs7412_2
gccagagcaccgaggagctgcrggtgmgcctcgcctcccacctgcgcaagctgyvtvagcggctcctccgcgatgccgatgacctgcagaagygcctggcagtgtaccaggccggggcccgcgagggcgccgagcgcggcctcagcgccatccgcgagcgcctggggcccctggtggaacagggccgcgtgcgggccgccactgtgggctccctggc

SEQ ID NO: 532 - DNA, 16 bp, Artificial Sequence
  Primer rs7412_t1_1728_F-sp
gcgaggtgcaggccat

SEQ ID NO: 533 - DNA, 16 bp, Artificial Sequence
  Primer rs7412_t1_1728_R-sp
acgcggccctgttcca

SEQ ID NO: 534 - DNA, 182 bp, Artificial Sequence
  Primer Amplicon rs7412_t1_1728
gcggacatggaggacgtgygcggccgcctggtgcagtaccgcggcgaggtgcaggccatgctcggccagagcaccgaggagctgcrggtgmgcctcgcctcccacctgcgcaagctgyvtvagcggctcctccgcgatgccgatgacctgcagaagygcctggcagtgtaccaggccggggc

SEQ ID NO: 535 - DNA, 17 bp, Artificial Sequence
  Primer rs7412_F2R2_F-sp
gccagagcaccgaggag

SEQ ID NO: 536 - DNA, 18 bp, Artificial Sequence
  Primer rs7412_F2R2_R-sp
ctgggcccgctcctgtag

SEQ ID NO: 537 - DNA, 245 bp, Artificial Sequence
  Amplicon rs7412_F2R2
gccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccag

SEQ ID NO: 538 - DNA, 17 bp, Artificial Sequence
  Primer rs7412_1430_F-sp
gccagagcaccgaggag

SEQ ID NO: 539 - DNA, 16 bp, Artificial Sequence
  Primer rs7412_1430_R-sp
gccagggagcccacag

SEQ ID NO: 540 - DNA, 211 bp, Artificial Sequence
  Amplicon rs7412_1430
gcgaggtgcaggccatgctcggccagagcaccgaggagctgcrggtgmgcctcgcctcccacctgcgcaagctgyvtvagcggctcctccgcgatgccgatgacctgcagaagygcctggcagtgtaccaggccggggcccgcgagggcgccgagcgcggcctcagcgccatccgcgagcgcctggggcccctggtggaacagggccgcgt

SEQ ID NO: 541 - DNA, 23 bp, Artificial Sequence
  Primer rs7501331-r2-1_1849_F-sp
gccaagatgacatgtattcaagc

SEQ ID NO: 542 - DNA, 23 bp, Artificial Sequence
  Primer rs7501331-r2-1_1849_R-sp
tcaggacaagaggatgcatttta

SEQ ID NO: 543 - DNA, 214 bp, Artificial Sequence
  Amplicon rs7501331-r2-1_1849
atgtttgctctggattacacgtttttattttactttttgaaaactgaattttttcagaatgcagaagtgggcacaaatttaatcaaagtggyatctacaacagccacggccctgaaggaagaagatggccaagtctactgccagccggaatttctttatgaaggtaaaatgcatcctcttgtcctgagtttaggaaaggggcagattttcacag

SEQ ID NO: 544 - DNA, 23 bp, Artificial Sequence
  Primer rs7501331_1594_F-sp
atgtttgctctggattacacgtt

SEQ ID NO: 545 - DNA, 23 bp, Artificial Sequence
  Primer rs7501331_1594_R-sp
ctgtgaaaatctgcccctttcct

SEQ ID NO: 546 - DNA, 238 bp, Artificial Sequence
  Amplicon rs7501331_1594
gccaagatgacatgtattcaagcatttyaaaaaatatatacactaaagcaaatgtttgctctggattacacgtttttattttactttttgaaaactgaattttttcagaatgcagaagtgggcacaaatttaatcaaagtggyatctacaacagccacggccctgaaggaagaagatggccaagtctactgccagccggaatttctttatgaaggtaaaatgcatcctcttgtcctga

SEQ ID NO: 547 - DNA, 20 bp, Artificial Sequence
  Primer rs7594220_t1_1877_F-sp
taaaagggtgaggtggagcc

SEQ ID NO: 548 - DNA, 17 bp, Artificial Sequence
  Primer rs7594220_t1_1877_R-sp
tccccaccccatggtag

SEQ ID NO: 549 - DNA, 180 bp, Artificial Sequence
  Amplicon rs7594220_t1_1877
cactcagccccgccattcgagaatccagttcccctccaccttccactttgtatccasgtaaaaatraayacaarggggactggcctggggaggaaaggacccagaagaagagagaggatgagaaagggcctcggcattgccaggaatgttgagaacaggcgggaggtaagcacagaggtg

SEQ ID NO: 550 - DNA, 18 bp, Artificial Sequence
  Primer rs7594220_1230_F-sp
cactcagccccgccattc

SEQ ID NO: 551 - DNA, 20 bp, Artificial Sequence
  Primer rs7594220_1230_R-sp
cacctctgtgcttacctccc

SEQ ID NO: 552 - DNA, 232 bp, Artificial Sequence
  Amplicon rs7594220_1230
taaaagggtgaggtggagccaccccactcagccccgccattcgagaatccagttcccctccaccttccactttgtatccasgtaaaaatraayacaarggggactggcctggggaggaaaggacccagaagaagagagaggatgagaaagggcctcggcattgccaggaatgttgagaacaggcgggaggtaagcacagaggtggmtgaagggggctaccatggggtgggga

SEQ ID NO: 553 - DNA, 23 bp, Artificial Sequence
  Primer rs763035-r3-1_4_F-sp
tgctcttttcatttggttgatct

SEQ ID NO: 554 - DNA, 26 bp, Artificial Sequence
  Primer rs763035-r3-1_4_R-sp
tctttgcattctccttatttgtactt

SEQ ID NO: 555 - DNA, 192 bp, Artificial Sequence
  Amplicon rs763035-r3-1_4
attaaaatccaagaggctaacatttaattaatgacatttaagatcttctatatyggataatgctatacattatattagrtttaatatttctrttaartatagatttagtaaattactaaaaatgctaaaaattcatcaaatatatatgtaagtacaaataagraraatgcaaagagagatattagaaagggg

SEQ ID NO: 556 - DNA, 24 bp, Artificial Sequence
  Primer rs763035_1_F-sp
attaaaatccaagaggctaacatt

SEQ ID NO: 557 - DNA, 24 bp, Artificial Sequence
  Primer rs763035_1_R-sp
cccctttctaatatctctctttgc

SEQ ID NO: 558 - DNA, 231 bp, Artificial Sequence
  Amplicon rs763035_1
tgctcttttcatttggttgatcttcatgtattttcacattattaagctttatataaattaaaatccaagaggctaacatttaattaatgacatttaagatcttctatatcggataatgctatacattatattaggtttaatatttctattaagtatagatttagtaaattactaaaaatgctaaaaattcatcaaatatatatgtaagtacaaataaggagaatgcaaaga

SEQ ID NO: 559 - DNA, 18 bp, Artificial Sequence
  Primer rs7787362_1557_F-sp
cgtgtgtgcaggcatgtg

SEQ ID NO: 560 - DNA, 18 bp, Artificial Sequence
  Primer rs7787362_1557_R-sp
cacggcggcttgagaaag

SEQ ID NO: 561 - DNA, 218 bp, Artificial Sequence
  Primer Amplicon rs7787362_1557
cgtgtgtgcaggcatgtgcgtgcaggtgtacatgtggaggcatgtgtggtcaggcatgcatgtgcaggtgtgtgcgtgtgcaggtgtgcgtgtgcatgaacatgactgtaaactgggygccctttccccttcaagcctgtgggtctcagtccagagtccacagacagcgttagagccttgcctgtgactcagaggcaggcctttctcaagccgccgtg

SEQ ID NO: 562 - DNA, 18 bp, Artificial Sequence
  Primer rs7787362_t1_1826_F-sp
aggtgtgtgcgtgtgcag

SEQ ID NO: 563 - DNA, 18 bp, Artificial Sequence
  Primer rs7787362_t1_1826_R-sp
cacatggcccagacctcc

SEQ ID NO: 564 - DNA, 185 bp, Artificial Sequence
  Amplicon rs7787362_t1_1826
aggtgtgtgcgtgtgcaggtgtgcgtgtgcatgaacatgactgtaaactgggygccctttccccttcaagcctgtgggtctcagtccagagtccacagacagcgttagagccttgcctgtgactcagaggcaggcctttctcaagccgccgtgccgccgggtcggggggaggtctgggccatgtg

SEQ ID NO: 565 - DNA, 19 bp, Artificial Sequence
  Primer rs885479_1966_F-sp
cctgcagctccatgctgtc

SEQ ID NO: 566 - DNA, 23 bp, Artificial Sequence
  Primer rs885479_1966_R-sp
atagccaggaagaagaccacgag

SEQ ID NO: 567 - DNA, 226 bp, Artificial Sequence
  Amplicon rs885479_1966
cctgcagctccatgctgtccagcctctgcttcctgggcgccatcgccgtggaccgctacatctccatcttctacgcactgbgctaccacagcatcgtgaycctgmcgygggcgcggcragccgttgcggccatytgggtgkccagtgtcgtcttcagcacgctcttcatcgcctactacgaccacgtggccgtcctgctgtgcctcgtggtcttcttcctggctat

SEQ ID NO: 568 - DNA, 23 bp, Artificial Sequence
  Primer rs885479_2_F-sp
ccgctacatctccatcttctacg

SEQ ID NO: 569 - DNA, 19 bp, Artificial Sequence
  Primer rs885479_2_R-sp
ggccatgagcaccagcata

SEQ ID NO: 570 - DNA, 190 bp, Artificial Sequence
  Amplicon rs885479_2
ccgctacatctccatcttctacgcactgbgctaccacagcatcgtgaycctgmcgygggcgcggcragccgttgcggccatytgggtgkccagtgtcgtcttcagcacgctcttcatcgcctactacgaccacgtggccgtcctgctgtgcctcgtggtcttcttcctggctatgctggtgctcatggcc

SEQ ID NO: 571 - DNA, 18 bp, Artificial Sequence
  Primer rs885479_3_F-sp
tgcagcagctggacaatg

SEQ ID NO: 572 - DNA, 20 bp, Artificial Sequence
  Primer rs885479_3_R-sp
tggtcgtagtaggcgatgaa

SEQ ID NO: 573 - DNA, 217 bp, Artificial Sequence
  Amplicon rs885479_3
tgcagcagctggacaatgtcattgacgtgatcacctgcagctccatgctgtccagcctctgcttcctgggcgccatcgccgtggaccgctacatctccatcttctacgcactgcgctaccacagcatcgtgaccctgccgcgggcgcggcgagccgttgcggccatctgggtggccagtgtcgtcttcagcacgctcttcatcgcctactacgacca